(19) United States
(12) Patent Application Publication
(10) Pub. No.: US 2023/0251273 A1
(43) Pub. Date: Aug. 10, 2023

(54) METHOD FOR DETECTING INFLAMMASOME PROTEINS AS BIOMARKERS OF NEUROLOGICAL DISORDERS

(71) Applicant: University of Miami, Miami, FL (US)

(72) Inventors: Juan Pablo DE RIVERO VACCARI, Miami, FL (US); Robert W. KEANE, Miami, FL (US); W. Dalton DIETRICH, Miami, FL (US); Helen BRAMLETT, Miami, FL (US)

(21) Appl. No.: 17/812,058

(22) Filed: Jul. 12, 2022

Related U.S. Application Data
(63) Continuation of application No. 16/648,839, filed on Mar. 19, 2020, now abandoned.
(60) Provisional application No. 62/696,549, filed on Jul. 11, 2018.
(60) Provisional application No. 62/560,963, filed on Sep. 20, 2017.

(51) Int. Cl. G01N 33/68; A61K 31/136; A61K 31/137; A61K 31/225; A61K 31/277; A61K 38/02; A61K 38/21; A61K 39/395

(52) U.S. Cl. CPC G01N 33/6893; A61K 31/136; A61K 31/137; A61K 31/225; A61K 31/277; A61K 38/02; A61K 38/215; A61K 39/3955; G01N 33/6896; G01N 2333/54; G01N 2333/545; G01N 2333/96469; G01N 2800/285; G01N 2800/2871

(57) ABSTRACT

The present invention provides compositions and methods for detecting components of the inflammasome in a sample from a subject as markers for brain injuries such as multiple sclerosis, stroke or traumatic brain injury. Methods of using such inflammasome markers to determine prognosis, direct treatment and monitor response to treatment for the subject with a brain injury such as multiple sclerosis, stroke, mild cognitive impairment or traumatic brain injury are also described.

### CROSS-REFERENCE TO RELATED APPLICATIONS

This application is a continuation of U.S. Application No. 16/648,839, filed on Mar. 19, 2020, which is a national phase of International Application No. PCT/US2018/051899, filed Sep. 20, 2018, which claims the benefit of priority to U.S. Provisional Application No. 62/696,549 filed Jul. 11, 2018, and U.S. Provisional Application No. 62/560,963 filed Sep. 20, 2017, the contents of each of which are hereby is incorporated by reference in its their entirety for all purposes.

### STATEMENT AS TO FEDERALLY SPONSORED RESEARCH

This invention was made with U.S. government support under grant numbers 5R42NS086274-03 and NS086274 awarded by the National Institute of Health. The U.S. government has certain rights in the invention.

### FIELD

The invention relates generally to the fields of immunology and medicine. More particularly, the invention relates to compositions and methods for detecting ASC (Apoptosis-associated Speck-like protein containing a Caspase Activating Recruitment Domain (CARD)) activity, caspase-1, IL-18, IL-1β, NOD-like receptors (NLR) and Absent in Melanoma 2 (AIM2)-like receptors (ALR) and other inflammasome proteins in samples obtained from a mammal as biomarkers for neurological disorders such as multiple sclerosis (MS), stroke, mild cognitive impairment (MCI) or traumatic brain injury (TBI).

### REFERENCE TO AN ELECTRONIC SEQUENCE LISTING

The contents of the electronic sequence listing (UNMI_014_03US_SeqList_ST26.xml; Size: 4,580 bytes; and Date of Creation: Nov. 3, 2022) are herein incorporated by reference in its entirety.

### BACKGROUND

Multiple sclerosis (MS) is a progressive autoimmune disorder that affects the central nervous system (CNS). Pathologically, it is characterized by demyelination in the spinal cord and brain as well as the presence of inflammatory lesions (Compston A. The pathogenesis and basis for treatment in multiple sclerosis. Clin Neurol Neurosurg. 2004;106:246-8). Clinically, patients with MS present blurred vision, muscle weakness, fatigue, dizziness, as well as balance and gate problems (Compston A. The pathogenesis and basis for treatment in multiple sclerosis. Clin Neurol Neurosurg. 2004;106:246-8). In the United States, alone, there are 400,000 patients with MS and about 2 million patients worldwide (Compston A. The pathogenesis and basis for treatment in multiple sclerosis. Clin Neurol Neurosurg. 2004;106:246-8).

Since the 1960s immunoglobulin (Ig) G oligoclonal bands (OCB) have been used as a classic biomarker in the diagnosis of MS (Stangel M, Fredrikson S, Meinl E, Petzold A, Stuve O and Tumani H. The utility of cerebrospinal fluid analysis in patients with multiple sclerosis. Nat Rev Neurol. 2013;9:267-76). However, the specificity of IgG-OCB is only 61%, as a result, other diagnostic criteria is needed to clinically determine the diagnosis of MS (Teunissen CE, Malekzadeh A, Leurs C, Bridel C and Killestein J. Body fluid biomarkers for multiple sclerosis--the long road to clinical application. Nat Rev Neurol. 2015;11:585-96), yet CSF-restricted IgG-OCB is a good predictor for conversion from CIS to CDMS, independently of MRI (Tintore M, Rovira A, Rio J, Tur C, Pelayo R, Nos C, Tellez N, Perkal H, Comabella M, Sastre-Garriga J and Montalban X. Do oligoclonal bands add information to MRI in first attacks of multiple sclerosis? Neurology. 2008;70:1079-83). Similar results have been obtained when analyzing IgM-OCB (Villar LM, Masjuan J, Gonzalez-Porque P, Plaza J, Sadaba MC, Roldan E, Bootello A and Alvarez-Cermeno JC. Intrathecal IgM synthesis predicts the onset of new relapses and a worse disease course in MS. Neurology. 2002;59:555-9). An important area of research in the field of MS is the identification of suitable biomarkers to predict who is at risk of developing MS, biomarkers of disease progression or exacerbation, as well as biomarkers of treatment response and prognosis.

There are 17.5 million deaths related to cardiovascular disease every year, of which 6.7 million occur as a result of stroke.( Mendis S, Davis S and Norrving B. Organizational update: the world health organization global status report on noncommunicable diseases 2014; one more landmark step in the combat against stroke and vascular disease. Stroke. 2015;46:e121-2). Even though there have been some large studies of stroke biomarkers, there is yet to be a gold standard biomarker that is used in the care of stroke patients. There is still a need for a biomarker that offers high sensitivity and high specificity for stroke.

The US Center for Disease Control (“CDC) defines a traumatic brain injury (“TBI”) “as a disruption in the normal function of the brain that can be caused by a bump, blow, or jolt to the head, or penetrating head injury.” As of 2010, the CDC recorded 823.7 TBI-related emergency room visits, hospitalizations and deaths per 100,000 individuals in the US. (US Centers for Disease Control “Traumatic Brain Injury and Concussion Website. https://www.cdc.gov/traumaticbraininjury/index.html (as of 21 Jun. 2018)). An important area of research in the field of TBI is the identification of suitable biomarkers to at risk of developing TBI, biomarkers of disease diagnosis, progression or exacerbation, as well as biomarkers of treatment response and prognosis. Previous work on the inflammasome has indicated that inflammasome proteins can be used as biomarkers after traumatic brain injury. The inflammasome is a multiprotein complex of the innate immune response involved in the activation of caspase-1 and the processing of the inflammatory cytokines IL-1beta and IL18. The inflammasome contributes to the inflammatory response after injury to the brain and the spinal cord, among others.

A great deal of interest has been generated concerning the topic of a boundary or transitional state between normal aging and dementia, or Alzheimer disease (AD). This condition has received several descriptors including mild cognitive impairment (MCI), incipient dementia, and isolated memory impairment. Subjects with a mild cognitive impairment (MCI) have a memory impairment beyond that expected for age and education yet are not demented. These subjects are becoming the focus of many prediction studies and early intervention trials. However, the diagnostic criteria for MCI has not generally been elucidated and the presence of biomarkers is lacking.

Thus, presented herein for addressing the above identified needs are inflammasome components useful as biomarkers with high sensitivity and specificity for various neurological or psychiatric conditions and methods of their use.

### SUMMARY

In one aspect, provided herein is a method of evaluating a patient suspected of having multiple sclerosis (MS), the method comprising: measuring the level of at least one inflammasome protein in a biological sample obtained from the patient; determining the presence or absence of a protein signature associated with MS, wherein the protein signature comprises an elevated level of the at least one inflammasome protein; and selecting the patient as having MS if the patient exhibits the presence of the protein signature. In some cases, the patient is presenting with clinical symptoms consistent with MS. In some cases, the MS is relapsing-remitting MS (RRMS), secondary-progressive MS (SPMS), primary-progressive MS (PPMS), or progressive-relapsing MS (PRMS). In some cases, the biological sample obtained from the patient is cerebrospinal fluid (CSF), CNS microdialysate, saliva, serum, plasma, urine or serum-derived extracellular vesicles (EVs). In some cases, the level of the at least one inflammasome protein in the protein signature is measured by an immunoassay utilizing one or more antibodies directed against the at least one inflammasome protein in the protein signature. In some cases, the at least one inflammasome protein is interleukin 18 (IL-18), IL-1beta, apoptosis-associated speck-like protein containing a caspase recruitment domain (ASC), caspase-1, or combinations thereof. In some cases, the at least one inflammasome protein comprises each of caspase-1, IL-18, IL-1beta and ASC. In some cases, the at least one inflammasome protein comprises ASC. In some cases, the antibody binds to the PYRIN-PAAD-DAPIN domain (PYD), C-terminal caspase-recruitment domain (CARD) domain or a portion of the PYD or CARD domain of the ASC protein. In some cases, the level of the at least one inflammasome protein in the protein signature is enhanced relative to the level of the at least one inflammasome protein in a biological sample obtained from a control. In some cases, the biological sample obtained from the control is cerebrospinal fluid (CSF), CNS microdialysate, saliva, serum, plasma, urine or serum-derived extracellular vesicles (EVs). In some cases, the control is a healthy individual, wherein the healthy individual is an individual not presenting with clinical symptoms consistent with MS. In some cases, the at least one inflammasome protein comprises ASC, wherein the level of ASC is at least 50% higher than the level of ASC in the biological sample obtained from a control. In some cases, the level of the at least one inflammasome protein in the protein signature is enhanced relative to a pre-determined reference value or range of reference values. In some cases, the biological sample obtained from patient is serum and the patient is selected as having MS with a sensitivity of at least 80%, 85%, 90%, 95%, 99% or 100% and a specificity of at least 90%. In some cases, the biological sample is serum and the patient is selected as having MS with a specificity of at least 80%, 85%, 90%, 95%, 99% or 100%. In some cases, the biological sample is serum and the patient is selected as having MS with a sensitivity of at least 90% and a specificity of at least 80%. In some cases, the at least one inflammasome protein comprises ASC. In some cases, a cut-off value for determining the sensitivity, specificity or both is selected from Table 7. In some cases, the sensitivity and/or sensitivity is determined using the area under curve (AUC) from receiver operator characteristic (ROC) curves with confidence intervals of 95%.

In another aspect, provided herein is a method of evaluating a patient suspected of having suffered a stroke, the method comprising: measuring the level of at least one inflammasome protein in a biological sample obtained from the patient; determining the presence or absence of a protein signature associated with stroke or a stroke-related injury, wherein the protein signature comprises an elevated level of the at least one inflammasome protein; and selecting the patient as having suffered from a stroke if the patient exhibits the presence of the protein signature. In some cases, the patient is presenting with clinical symptoms consistent with stroke, wherein the stroke is ischemic stroke, transient ischemic stroke or hemorrhagic stroke. In some cases, the biological sample obtained from the patient is cerebrospinal fluid (CSF), CNS microdialysate, saliva, serum, plasma, urine or serum-derived extracellular vesicles (EVs). In some cases, the level of the at least one inflammasome protein in the protein signature is measured by an immunoassay utilizing one or more antibodies directed against the at least one inflammasome protein in the protein signature. In some cases, the at least one inflammasome protein is interleukin 18 (IL-18), IL-1beta, apoptosis-associated speck-like protein containing a caspase recruitment domain (ASC), caspase-1, or combinations thereof. In some cases, the at least one inflammasome protein comprises each of caspase-1, IL-18, IL-1beta and ASC. In some cases, the at least one inflammasome protein comprises ASC. In some cases, the antibody binds to the PYRIN-PAAD-DAPIN domain (PYD), C-terminal caspase-recruitment domain (CARD) domain or a portion of the PYD or CARD domain of the ASC protein. In some cases, the level of the at least one inflammasome protein in the protein signature is enhanced relative to the level of the at least one inflammasome protein in a biological sample obtained from a control. In some cases, the biological sample obtained from the control is cerebrospinal fluid (CSF), CNS microdialysate, saliva, serum, plasma, urine or serum-derived extracellular vesicles (EVs). In some cases, the control is a healthy individual, wherein the healthy individual is an individual not presenting with clinical symptoms consistent with MS. In some cases, the at least one inflammasome protein comprises ASC, wherein the level of ASC in a serum sample obtained from the subject is at least 70% higher than the level of ASC in a serum sample obtained from a control. In some cases, the at least one inflammasome protein comprises ASC, wherein the level of ASC in a serum-derived EV sample obtained from the subject is at least 110% higher than the level of ASC in a serum-derived EV sample obtained from a control. In some cases, the level of the at least one inflammasome protein in the protein signature is enhanced relative to a pre-determined reference value or range of reference values. In some cases, the biological sample obtained from patient is serum and the patient is selected as having suffered a stroke with a sensitivity of at least 80%, 85%, 90%, 95%, 99% or 100% and a specificity of at least 90%. In some cases, the biological sample is serum and the patient is selected as having suffered a stroke with a specificity of at least 80%, 85%, 90%, 95%, 99% or 100%. In some cases, the biological sample is serum and the patient is selected as having suffered a stroke with a sensitivity of at least 100% and a specificity of at least 95%. In some cases, the at least one inflammasome protein comprises ASC. In some cases, a cut-off value for determining the sensitivity, specificity or both is selected from Table 8. In some cases, the biological sample obtained from patient is serum-derived EVs and the patient is selected as having suffered a stroke with a sensitivity of at least 80%, 85%, 90%, 95%, 99% or 100% and a specificity of at least 90%. In some cases, the biological sample is serum-derived EVs and the patient is selected as having suffered a stroke with a specificity of at least 80%, 85%, 90%, 95%, 99% or 100%. In some cases, the biological sample is serum-derived EVs and the patient is selected as having suffered a stroke with a sensitivity of at least 100% and a specificity of at least 100%. In some cases, the at least one inflammasome protein comprises ASC. In some cases, a cut-off value for determining the sensitivity, specificity or both is selected from Table 9. In some cases, the sensitivity and/or sensitivity is determined using the area under curve (AUC) from receiver operator characteristic (ROC) curves with confidence intervals of 95%.

In yet another aspect, provided herein is a method of treating a patient diagnosed with multiple sclerosis (MS), the method comprising administering a standard of care treatment for MS to the patient, wherein the diagnosis of MS was made by detecting an elevated level of at least one inflammasome protein in a biological sample obtained from the patient. In some cases, the MS is relapsing-remitting MS (RRMS), secondary-progressive MS (SPMS), primary-progressive MS (PPMS), or progressive-relapsing MS (PRMS). In some cases, the standard of care treatment is selected from therapies directed towards modifying disease outcome, managing relapses, managing symptoms or any combination thereof. In some cases, the therapies directed toward modifying disease outcome are selected from beta-interferons, glatiramer acetate, fingolimod, teriflunomide, dimethyl fumarate, mitoxanthrone, ocrelizumab, alemtuzumab, daclizumab and natalizumab.

In still another aspect, provided herein is a method of treating a patient diagnosed with stroke or a stroke related injury, the method comprising administering a standard of care treatment for stroke or stroke-related injury to the patient, wherein the diagnosis of stroke or stroke-related injury was made by detecting an elevated level of at least one inflammasome protein in a biological sample obtained from the patient. In some cases, the stroke is ischemic stroke, transient ischemic stroke or hemorrhagic stroke. In some cases, the stroke is ischemic stroke or transient ischemic stroke and the standard of care treatment is selected from tissue plasminogen activator (tPA), antiplatelet medicine, anticoagulants, a carotid artery angioplasty, carotid endarterectomy, intra-arterial thrombolysis and mechanical clot removal in cerebral ischemia (MERCI) or a combination thereof. In some cases, the stroke is hemorrhagic stroke and the standard of care treatment is an aneurysm clipping, coil embolization or arteriovenous malformation (AVM) repair. In some cases, the elevated level of the at least one inflammasome protein is measured by an immunoassay utilizing one or more antibodies directed against the at least one inflammasome protein. In some cases, the level of the at least one inflammasome protein is enhanced relative to the level of the at least one inflammasome protein in a control sample. In some cases, the level of the at least one inflammasome protein is enhanced relative to a pre-determined reference value or range of reference values. In some cases, the at least one inflammasome protein is interleukin 18 (IL-18), apoptosis-associated speck-like protein containing a caspase recruitment domain (ASC), caspase-1, or combinations thereof. In some cases, the at least one inflammasome protein is caspase-1, IL-18, and ASC. In some cases, the at least one inflammasome protein is ASC. In some cases, the antibody binds to the PYRIN-PAAD-DAPIN domain (PYD), C-terminal caspase-recruitment domain (CARD) domain or a portion of the PYD or CARD domain of the ASC protein. In some cases, the biological sample is cerebrospinal fluid (CSF), CNS microdialysate, saliva, serum, plasma, urine or serum-derived extracellular vesicles (EVs).

In a still further aspect, provided herein is a method of evaluating a patient suspected of having traumatic brain injury (TBI), the method comprising: measuring the level of at least one inflammasome protein in a biological sample obtained from the patient; determining the presence or absence of a protein signature associated with TBI, wherein the protein signature comprises an elevated level of the at least one inflammasome protein; and selecting the patient as having TBI if the patient exhibits the presence of the protein signature. In some cases, the patient is presenting with clinical symptoms consistent with TBI. In some cases, the biological sample obtained from the patient is cerebrospinal fluid (CSF), CNS microdialysate, saliva, serum, plasma, urine or serum-derived extracellular vesicles (EVs). In some cases, the level of the at least one inflammasome protein in the protein signature is measured by an immunoassay utilizing one or more antibodies directed against the at least one inflammasome protein in the protein signature. In some cases, the at least one inflammasome protein is interleukin 18 (IL-18), IL-1β, apoptosis-associated speck-like protein containing a caspase recruitment domain (ASC), caspase-1, or combinations thereof. In some cases, the at least one inflammasome protein comprises caspase-1. In some cases, the at least one inflammasome protein comprises ASC. In some cases, the antibody binds to the PYRIN-PAAD-DAPIN domain (PYD), C-terminal caspase-recruitment domain (CARD) domain or a portion of the PYD or CARD domain of the ASC protein. In some cases, the level of the at least one inflammasome protein in the protein signature is enhanced relative to the level of the at least one inflammasome protein in a biological sample obtained from a control. In some cases, the at least one inflammasome protein comprises caspase-1, wherein the level of caspase-1 is at least 50% higher than the level of caspase-1in the biological sample obtained from the control. In some cases, the at least one inflammasome protein comprises ASC, wherein the level of ASC is at least 50% higher than the level of ASC in the biological sample obtained from the control. In some cases, the biological sample obtained from the control is cerebrospinal fluid (CSF), CNS microdialysate, saliva, serum, plasma, urine or serum-derived extracellular vesicles (EVs). In some cases, the control is a healthy individual, wherein the healthy individual is an individual not presenting with clinical symptoms consistent with TBI. In some cases, the level of the at least one inflammasome protein in the protein signature is enhanced relative to a pre-determined reference value or range of reference values. In some cases, the biological sample obtained from patient is serum and the patient is selected as having TBI with a sensitivity of at least 80%, 85%, 90%, 95%, 99% or 100% and a specificity of at least 90%. In some cases, the biological sample is serum and the patient is selected as having TBI with a specificity of at least 80%, 85%, 90%, 95%, 99% or 100%. In some cases, the biological sample is serum and the patient is selected as having TBI with a sensitivity of at least 90% and a specificity of at least 80%. In some cases, the sensitivity and/or sensitivity is determined using the area under curve (AUC) from receiver operator characteristic (ROC) curves with confidence intervals of 95%. In some cases, the at least one inflammasome protein comprises ASC. In some cases, a cut-off value for determining the sensitivity, specificity or both is selected from Tables 11B, 12B, 14A, 16, 17 or 19. In some cases, the at least one inflammasome protein comprises caspase-1. In some cases, a cut-off value for determining the sensitivity, specificity or both is selected from Tables 11A or 15.

In yet another aspect, provided herein is a method of evaluating a patient suspected of having a brain injury, the method comprising: measuring the level of at least one inflammasome protein in a biological sample obtained from the patient; determining the presence or absence of a protein signature associated with brain injury, wherein the protein signature comprises an elevated level of the at least one inflammasome protein; and selecting the patient as having brain injury if the patient exhibits the presence of the protein signature. In some cases, the patient is presenting with clinical symptoms consistent with brain injury. In some cases, the biological sample obtained from the patient is cerebrospinal fluid (CSF), CNS microdialysate, saliva, serum, plasma, urine or serum-derived extracellular vesicles (EVs). In some cases, the level of the at least one inflammasome protein in the protein signature is measured by an immunoassay utilizing one or more antibodies directed against the at least one inflammasome protein in the protein signature. In some cases, the at least one inflammasome protein is interleukin 18 (IL-18), IL-1β, apoptosis-associated speck-like protein containing a caspase recruitment domain (ASC), caspase-1, or combinations thereof. In some cases, the at least one inflammasome protein comprises ASC. In some cases, the antibody binds to the PYRIN-PAAD-DAPIN domain (PYD), C-terminal caspase-recruitment domain (CARD) domain or a portion of the PYD or CARD domain of the ASC protein. In some cases, the at least one inflammasome protein comprises caspase-1. In some cases, the level of the at least one inflammasome protein in the protein signature is enhanced relative to the level of the at least one inflammasome protein in a biological sample obtained from a control. In some cases, the at least one inflammasome protein comprises ASC, wherein the level of ASC is at least 50% higher than the level of ASC in the biological sample obtained from the control. In some cases, the at least one inflammasome protein comprises caspase-1, wherein the level of caspase-1 is at least 50% higher than the level of caspase-1in the biological sample obtained from the control. In some cases, the biological sample obtained from the control is cerebrospinal fluid (CSF), CNS microdialysate, saliva, serum, plasma, urine or serum-derived extracellular vesicles (EVs). In some cases, the control is a healthy individual, wherein the healthy individual is an individual not presenting with clinical symptoms consistent with brain injury. In some cases, the brain injury is selected from a traumatic brain injury, stroke, mild cognitive impairment or multiple sclerosis. In some cases, the level of the at least one inflammasome protein in the protein signature is enhanced relative to a pre-determined reference value or range of reference values. In some cases, the brain injury is traumatic brain injury (TBI). In some cases, the biological sample obtained from patient is serum and the patient is selected as having TBI with a sensitivity of at least 80%, 85%, 90%, 95%, 99% or 100% and a specificity of at least 90%. In some cases, the biological sample is serum and the patient is selected as having TBI with a specificity of at least 80%, 85%, 90%, 95%, 99% or 100%. In some cases, the biological sample is serum and the patient is selected as having TBI with a sensitivity of at least 90% and a specificity of at least 80%. In some cases, the sensitivity and/or sensitivity is determined using the area under curve (AUC) from receiver operator characteristic (ROC) curves with confidence intervals of 95%. In some cases, the at least one inflammasome protein comprises ASC. In some cases, a cut-off value for determining the sensitivity, specificity or both is selected from Tables 11B, 12B, 14A, 16, 17 or 19. In some cases, the at least one inflammasome protein comprises caspase-1. In some cases, a cut-off value for determining the sensitivity, specificity or both is selected from Tables 11A or 15. In some cases, the brain injury is mid cognitive impairment (MCI). In some cases, the biological sample obtained from patient is serum and the patient is selected as having MCI with a sensitivity of at least 75%, 80%, 85%, 90%, 95%, 99% or 100%. In some cases, the biological sample is serum and the patient is selected as having MCI with a specificity of at least 55%, 60%, 65%, 70%, 75%, 80%, 85%, 90%, 95%, 99% or 100%. In some cases, the biological sample is serum and the patient is selected as having MCI with a sensitivity of at least 90% and a specificity of at least 70%. In some cases, the sensitivity and/or sensitivity is determined using the area under curve (AUC) from receiver operator characteristic (ROC) curves with confidence intervals of 95%. In some cases, the at least one inflammasome protein comprises ASC. In some cases, a cut-off value for determining the sensitivity, specificity or both is selected from Tables 22 or 23. In some cases, the at least one inflammasome protein comprises IL-18. In some cases, a cut-off value for determining the sensitivity, specificity or both is selected from Tables 22 or 25. In some cases, the brain injury is multiple sclerosis (MS). In some cases, the biological sample obtained from patient is serum and the patient is selected as having MS with a sensitivity of at least 80%, 85%, 90%, 95%, 99% or 100% and a specificity of at least 90%. In some cases, the biological sample is serum and the patient is selected as having MS with a specificity of at least 80%, 85%, 90%, 95%, 99% or 100%. In some cases, the biological sample is serum and the patient is selected as having MS with a sensitivity of at least 90% and a specificity of at least 80%. In some cases, the at least one inflammasome protein comprises ASC. In some cases, a cut-off value for determining the sensitivity, specificity or both is selected from Table 7. In some cases, the sensitivity and/or sensitivity is determined using the area under curve (AUC) from receiver operator characteristic (ROC) curves with confidence intervals of 95%. In some cases, the brain injury is stroke. In some cases, the biological sample obtained from patient is serum and the patient is selected as having suffered a stroke with a sensitivity of at least 80%, 85%, 90%, 95%, 99% or 100% and a specificity of at least 90%. In some cases, the biological sample is serum and the patient is selected as having suffered a stroke with a specificity of at least 80%, 85%, 90%, 95%, 99% or 100%. In some cases, the biological sample is serum and the patient is selected as having suffered a stroke with a sensitivity of at least 100% and a specificity of at least 95%. In some cases, the at least one inflammasome protein comprises ASC. In some cases, a cut-off value for determining the sensitivity, specificity or both is selected from Table 8. In some cases, the biological sample obtained from patient is serum-derived EVs and the patient is selected as having suffered a stroke with a sensitivity of at least 80%, 85%, 90%, 95%, 99% or 100% and a specificity of at least 90%. In some cases, the biological sample is serum-derived EVs and the patient is selected as having suffered a stroke with a specificity of at least 80%, 85%, 90%, 95%, 99% or 100%. In some cases, the biological sample is serum-derived EVs and the patient is selected as having suffered a stroke with a sensitivity of at least 100% and a specificity of at least 100%. In some cases, the at least one inflammasome protein comprises ASC. In some cases, a cut-off value for determining the sensitivity, specificity or both is selected from Table 9. In some cases, the sensitivity and/or sensitivity is determined using the area under curve (AUC) from receiver operator characteristic (ROC) curves with confidence intervals of 95%.

In a still further aspect, provided herein is a method of evaluating a patient suspected of having mild cognitive impairment (MCI) the method comprising: measuring the level of at least one inflammasome protein in a biological sample obtained from the patient; determining the presence or absence of a protein signature associated with MCI, wherein the protein signature comprises an elevated level of the at least one inflammasome protein; and selecting the patient as having MCI if the patient exhibits the presence of the protein signature. In some cases, the patient is presenting with clinical symptoms consistent with MCI. In some cases, the biological sample obtained from the patient is cerebrospinal fluid (CSF), CNS microdialysate, saliva, serum, plasma, urine or serum-derived extracellular vesicles (EVs). In some cases, the level of the at least one inflammasome protein in the protein signature is measured by an immunoassay utilizing one or more antibodies directed against the at least one inflammasome protein in the protein signature. In some cases, the at least one inflammasome protein is interleukin 18 (IL-18), IL-1β, apoptosis-associated speck-like protein containing a caspase recruitment domain (ASC), caspase-1, or combinations thereof. In some cases, the at least one inflammasome protein comprises ASC. In some cases, the at least one inflammasome protein comprises IL-18. In some cases, the antibody binds to the PYRIN-PAAD-DAPIN domain (PYD), C-terminal caspase-recruitment domain (CARD) domain or a portion of the PYD or CARD domain of the ASC protein. In some cases, the level of the at least one inflammasome protein in the protein signature is enhanced relative to the level of the at least one inflammasome protein in a biological sample obtained from a control. In some cases, the at least one inflammasome protein comprises ASC, wherein the level of ASC is at least 50% higher than the level of ASC in the biological sample obtained from the control. In some cases, the at least one inflammasome protein comprises IL-18, wherein the level of IL-18 is at least 25% higher than the level of IL-18 in the biological sample obtained from the control.

### BRIEF DESCRIPTION OF THE DRAWINGS

FIGS. 1A-1D illustrates that inflammasome proteins are elevated in the serum of MS patients. Protein levels in pg/ml of caspase-1 (FIG. 1A), ASC (FIG. 1B), IL-1β (FIG. 1C) and IL-18 (FIG. 1D) in serum samples from patients with MS and healthy donors. p-value of significance is shown above each box plot. Box and whiskers are shown for the 5^{th} and 95^{th} percentile. Caspase-1: N=9 control and 19 MS; ASC: N=115 control and 32 MS; IL-1β: N=21 control and 8 MS; and IL-18: N=119 control and 32 MS.

FIGS. 2A-2D illustrates ROC curves for caspase-1 (FIG. 2A), ASC (FIG. 2B), IL-1β (FIG. 2C) and IL-18 (FIG. 2D) from serum samples of MS and healthy donors.

FIG. 3 illustrates inflammasome proteins in serum as biomarkers of MS. ROC curves for caspase-1, ASC, IL-1beta and IL-18. Caspase-1: N=9 control and 19 MS; ASC: N=115 control and 32 MS; IL-1beta: N=21 control and 8 MS; and IL-18: N=119 control and 32 MS.

FIG. 4 illustrates a table containing the characteristics of the subjects with Multiple Sclerosis (MS) from Example 1.

FIGS. 5A-5D illustrates inflammasome proteins are elevated in the serum of stroke patients. Protein levels in pg/ml of caspase-1 (FIG. 5A), ASC (FIG. 5B), IL-1beta (FIG. 5C) and IL-18 (FIG. 5D) in serum samples from patients with stroke and healthy donors. p-value of significance is shown above each box plot. Box and whiskers are shown for the 5th and 95th percentile. N.S.= Not Significant. Caspase-1: N=8 control and 13 stroke; ASC: N=75 control and 16 stroke; IL-1beta: N=9 control and 8 stroke; and IL-18: N=79 control and 15 stroke.

FIG. 6 illustrates inflammasome proteins in serum as biomarkers of stroke. ROC curves for caspase-1, ASC, IL-1beta and IL-18. Caspase-1: N=8 control and 13 stroke; ASC: N=75 control and 16 stroke; IL-1beta: N=9 control and 8 stroke; and IL-18: N=79 control and 15 stroke.

FIG. 7A illustrates a comparison of total protein levels from serum-derived extracellular vesicle (EV). A Bradford Assay was carried following EV isolation from serum to determine total protein concentration in isolates with the Invitrogen kit (INVTR) and the ExoQuick kit (EQ). Data presented as mean+/-SEM. N= 6 per group. FIG. 7B depicts a representative image of total protein loaded. Stain-free image of serum-derived EV proteins. Equal amounts of protein lysates (10 ml) were loaded in each lane of a Criterion gel. FIG. 7C depicts a bar graph shows quantification of the entire lane corresponding to loaded EV isolated with the Invitrogen kit (INV) and the ExoQuick kit (EQ).

FIGS. 8A-8F illustrates EV characterization in serum from stroke patients. FIG. 8A depicts a representative immunoblot of CD81 and NCAM positive EV isolated with the Invitrogen Kit (IN) and the ExoQuick Kit (EQ). +Contr: Positive control of isolated EV. Quantification of CD81- (FIG. 8B) and NCAM- (FIG. 8C) positive EV isolated from serum with the Invitrogen kit (INV) and the ExoQuick kit (EQ). FIG. 8D depicts a electron microscopy image of EV isolated by two different techniques. Bar= 100 nm. Nanoparticle tracking analysis/particle size distribution of isolated serum-derived EV. Nanoparticle tracking analysis predicts size distribution and concentration of particles in serum-derived EV samples isolated with the Invitrogen kit (FIG. 8E) and the ExoQuick kit (FIG. 8F).

FIGS. 9A-9C illustrates that ASC is elevated in serum-derived EV of stroke patients. Protein levels in pg/ml of ASC (FIG. 9A), IL-1beta (FIG. 9B) and IL-18 (FIG. 9C) in serum-derived EV from patients with stroke and healthy donors. p-value of significance is shown above each box plot. Box and whiskers are shown for the 5th and 95th percentile. N.S.= Not Significant. ASC: N=16 control and 16 stroke; IL-1beta: N=10 control and 9 stroke; and IL-18: N=16 control and 13 stroke.

FIG. 10 illustrates Inflammasome proteins in serum-derived EV as biomarkers of stroke. ROC curves for ASC, IL-1beta and IL-18. ASC: N=16 control and 16 stroke; IL-1beta: N=10 control and 9 stroke; and IL-18: N=16 control and 13 stroke.

FIG. 11 illustrates a table containing the characteristics of the subjects with stroke from Example 2.

FIGS. 12A-12D illustrates ROC curves for caspase-1 (FIG. 12A), ASC (FIG. 12B), IL-1beta (FIG. 12C) and IL-18 (FIG. 12D) from serum samples of stroke and healthy donors.

FIGS. 13A-13F illustrates the characterization of inflammasome proteins in serum-derived EV. FIG. 13A depicts a representative image of immunoblot analyses of inflammasome proteins in EV from serum. Quantification of immunoblot analysis of (FIG. 13B) NLRP3, (FIG. 13C) caspase-1, (FIG. 13D) ASC, (FIG. 13E) IL-1beta, and (FIG. 13F) IL-18 in EV derived from serum using the Invitrogen kit (IN) and the ExoQuick kit (EQ). Data presented as mean+/-SEM. N= 6 per group. * p < 0.05.

FIGS. 14A-14C illustrates ROC curves for ASC (FIG. 14A), IL-1beta (FIG. 14B) and IL-18 (FIG. 14C) from serum-derived extracellular vesicles of stroke and healthy donors.

FIGS. 15A-15D illustrates how inflammasome proteins are elevated in the serum of TBI patients. Protein levels in pg/ml of ASC (FIG. 15A), caspase-1 (FIG. 15B), IL-18 (FIG. 15C) and IL-1β (FIG. 15D) in serum samples from patients with TBI and healthy donors (controls). ASC: N=120 control, 20 TBI. Caspase-1: N=11 control 19, TBI. IL-18: N=120 control, 21 TBI. IL-1β: N=25 control, 10 TBI. Box and whiskers are shown for the 5th and 95^{th} percentile. * p < 0.05.

FIGS. 16A-16D illustrates ROC curves for caspase-1 (FIG. 16A), ASC (FIG. 16B), IL-1β (FIG. 16C) and IL-18 (FIG. 16D) from serum samples of TBI patients and healthy donors.

FIGS. 17A-17B illustrates how inflammasome proteins are elevated in the CSF of TBI patients. Protein levels in pg/ml of ASC (FIG. 17A) and IL-18 (FIG. 17B) in CSF samples from patients with TBI and healthy donors (controls). ASC: N=21 control, 15 TBI. IL-18: N=24 control, 16 TBI. Box and whiskers are shown for the 5^{th} and 95^{th} percentile. * p < 0.05.

FIGS. 18A-18B illustrates ROC curves for ASC (FIG. 18A) and IL-18 (FIG. 18B) from CSF samples of TBI patients and healthy donors.

FIGS. 19A-19C illustrates inflammasome proteins as prognostic biomarkers of TBI. Protein levels in pg/ml of caspase-1 (FIG. 19A), ASC (FIG. 19B), and IL-18 (FIG. 19C) in serum samples from patients with TBI. Groups were divided into favorable and unfavorable outcomes based on the GOSE. p-value of significance is shown above each box plot. Box and whiskers are shown for the 5th and 95th percentile. Caspase-1: N=4 favorable and 16 unfavorable ASC: N=5 favorable and 16 unfavorable; and IL-18: N=5 favorable and 16 unfavorable.

FIGS. 20A-20B illustrates ROC curves for ASC outcomes (Favorable vs. Unfavorable) for the 2^{nd} (FIG. 20A) and 4^{th} (FIG. 20B) collection.

FIGS. 21A-21D illustrates inflammasome proteins are elevated in the serum of MCI patients. Protein levels in pg/ml of ASC (FIG. 21A), caspase-1 (FIG. 21B), IL-18 (FIG. 21C) and IL-1beta (FIG. 21D) in serum samples from patients with MCI and age-matched healthy donors (control). p-value of significance is shown above each box plot.

FIGS. 22A-22D illustrates ROC curves for ASC (FIG. 22A), caspase-1 (FIG. 22B), IL-18 (FIG. 22C) and IL-1beta (FIG. 22D) from serum samples of MCI and age-matched healthy donors.

FIG. 23 illustrates inflammasome proteins in serum as biomarkers of MCI. The ROC curves for caspase-1, ASC, IL-1beta and IL-18 from FIGS. 22A-22D are superimposed onto a single graph.

### DETAILED DESCRIPTION

### Definitions

Unless otherwise defined, all technical terms used herein have the same meaning as commonly understood by one of ordinary skill in the art to which this invention belongs.

As used herein, “protein” and “polypeptide” are used synonymously to mean any peptide-linked chain of amino acids, regardless of length or post-translational modification, e.g., glycosylation or phosphorylation.

By the terms “Apoptosis-associated Speck-like protein containing a Caspase Activating Recruitment Domain (CARD)” and “ASC” is meant an expression product of an ASC gene or isoforms thereof, or a protein that shares at least 65%, 75%, 80%, 85%, 90%, 95%, 96%, 97%, 98%, or 99% amino acid sequence identity with ASC (e.g., NP_037390 (Q9ULZ3-1), NP_660183 (Q9ULZ3-2) or Q9ULZ3-3 in human or NP_758825 (BAC43754) in rat) and displays a functional activity of ASC. A “functional activity” of a protein is any activity associated with the physiological function of the protein. Functional activities of ASC include, for example, recruitment of proteins for activation of caspase-1 and initiation of cell death.

By the term “ASC gene,” or “ASC nucleic acid” is meant a native ASC-encoding nucleic acid sequence, genomic sequences from which ASC cDNA can be transcribed, and/or allelic variants and homologues of the foregoing. The terms encompass double-stranded DNA, single-stranded DNA, and RNA.

As used herein, the term “inflammasome” means a multi-protein (e.g., at least two proteins) complex that activates caspase-1. Further, the term “inflammasome” can refer to a multi-protein complex that activates caspase-1 activity, which in turn regulates IL-1β, IL-18 and IL-33 processing and activation. See Arend et al. 2008; Li et al. 2008; and Martinon et al. 2002, each of which is incorporated by reference in their entireties. The terms “NLRP 1 inflammasome”, “NALP 1 inflammasome”, “NLRP2 inflammasome”, “NALP2 inflammasome”, “NLRP3 inflammasome”, “NALP3 inflammasome”, “NLRC4 inflammasome”, “IPAF inflammasome” or “AIM2 inflammasome” mean a protein complex of at least caspase-1 and one adaptor protein, e.g., ASC. For example, the terms “NLRP1 inflammasome” and “NALP1 inflammasome” can mean a multiprotein complex containing NLRP1, ASC, caspase-1, caspase-11, XIAP, and pannexin-1 for activation of caspase-1 and processing of interleukin-1β, interleukin-18 and interleukin-33. The terms “NLRP2 inflammasome” and “NALP2 inflammasome” can mean a multiprotein complex containing NLRP2 (aka NALP2), ASC and caspase-1,while the terms “NLRP3 inflammasome” and “NALP3 inflammasome” can mean a multiprotein complex containing NLRP3 (aka NALP3), ASC and the terms “NLRC4 inflammasome” and “IPAF inflammasome” can mean a multiprotein complex containing NLRC4 (aka IPAF), ASC and caspase-1. Additionally, the term “AIM2 Inflammasome” can mean a multiprotein complex comprising AIM2, ASC and caspase-1.

As used herein, the phrase “sequence identity” means the percentage of identical subunits at corresponding positions in two sequences (e.g., nucleic acid sequences, amino acid sequences) when the two sequences are aligned to maximize subunit matching, i.e., taking into account gaps and insertions. Sequence identity can be measured using sequence analysis software (e.g., Sequence Analysis Software Package from Accelrys CGC, San Diego, CA).

By the phrases “therapeutically effective amount” and “effective dosage” is meant an amount sufficient to produce a therapeutically (e.g., clinically) desirable result; the exact nature of the result will vary depending on the nature of the disorder being treated. For example, where the disorder to be treated is SCI, the result can be an improvement in motor skills and locomotor function, a decreased spinal cord lesion, etc. The compositions described herein can be administered from one or more times per day to one or more times per week. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions of the invention can include a single treatment or a series of treatments.

As used herein, the term “treatment” is defined as the application or administration of a therapeutic agent described herein, or identified by a method described herein, to a patient, or application or administration of the therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease, or the predisposition toward disease.

The terms “patient” “subject” and “individual” are used interchangeably herein, and mean a mammalian subject to be treated, such as, for example, human patients. In some cases, the methods of the invention find use in experimental animals, in veterinary applications, and in the development of animal models for disease, including, but not limited to, rodents including mice, rats, and hamsters, as well as primates.

As interchangeably used herein, “Absent in Melanoma 2” and “AIM2” can mean an expression product of an AIM2 gene or isoforms; or a protein that shares at least 65%, 75%, 80%, 85%, 90%, 95%, 96%, 97%, 98%, or 99% amino acid sequence identity with AIM2 (e.g., accession number(s) NX_014862, NP004824, XP016858337, XP005245673, AAB81613, BAF84731, AAH10940) and displays a functional activity of AIM2.

As interchangeably used herein, “NALP1” and “NLRP1” mean an expression product of an NALP1 or NLRP1 gene or isoforms; or a protein that shares at least 65%, 75%, 80%, 85%, 90%, 95%, 96%, 97%, 98%, or 99% amino acid sequence identity with NALP1 (e.g., accession number(s) AAH51787, NP_001028225, NP_127500, NP_127499, NP_127497, NP055737) and displays a functional activity of NALP1.

As interchangeably used herein, “NALP2” and “NLRP2” mean an expression product of an NALP2 or NLRP2 gene or isoforms; or a protein that shares at least 65%,, 75%, 80%, 85%, 90%, 95%, 96%, 97%, 98%, or 99% amino acid sequence identity with NALP2 (e.g., accession number(s) NP_001167552, NP_001167553, NP_001167554 or NP_060322) and displays a functional activity of NALP2.

As interchangeably used herein, “NALP3” and “NLRP3” mean an expression product of an NALP3 or NLRP3 gene or isoforms; or a protein that shares at least 65%, 75%, 80%, 85%, 90%, 95%, 96%, 97%, 98%, or 99%amino acid sequence identity with NALP3 (e.g., accession number(s) NP_001073289, NP_001120933, NP_001120934, NP_001230062, NP_004886, NP_899632, XP_011542350, XP_016855670, XP_016855671, XP_016855672 or XP_016855673) and displays a functional activity of NALP3.

As interchangeably used herein, “NLRC4” and “IPAF” mean an expression product of an NLRC4 or IPAF gene or isoforms; or a protein that shares at least 65%, 75%, 80%, 85%, 90%, 95%, 96%, 97%, 98%, or 99% amino acid sequence identity with NLRC4 (e.g., accession number(s) NP_001186067, NP001186068, NP_001289433 or NP_067032) and displays a functional activity of NLRC4.

By the term “stroke” and “ischemic stroke” is meant when blood flow is interrupted to part of the brain or spinal cord. By the term “ischemic stroke” and “transient ischemic stroke” is meant when blood flow is interrupted to part of the brain or spinal cord by blockage of an artery that supplies oxygen-rich blood to the brain or spinal cord. By the term “hemorrhagic stroke” is meant when blood flow is interrupted to part of the brain or spinal cord when an artery in the brain or spinal cord leaks blood or ruptures.

By “traumatic injury to the CNS” is meant any insult to the CNS from an external mechanical force, possibly leading to permanent or temporary impairments of CNS function.

The term “antibody” is meant to include polyclonal antibodies, monoclonal antibodies (mAbs), chimeric antibodies, humanized antibodies, anti-idiotypic (anti-Id) antibodies to antibodies that can be labeled in soluble or bound form, as well as fragments, regions or derivatives thereof, provided by any known technique, such as, but not limited to, enzymatic cleavage, peptide synthesis or recombinant techniques. Such anti-ASC and anti-NLRP1 antibodies of the present invention are capable of binding portions of ASC and NLRP1, respectively, which interfere with caspase-1 activation.

Methods involving conventional molecular biology techniques are described herein. Such techniques are generally known in the art and are described in detail in methodology treatises such as Molecular Cloning: A Laboratory Manual, 3rd ed., vol. 1-3, ed. Sambrook et al., Cold Spring Harbor Laboratory Press, Cold Spring Harbor, N.Y., 2001; and Current Protocols in Molecular Biology, ed. Ausubel et al., Greene Publishing and Wiley-Interscience, New York, 1992 (with periodic updates). Immunology techniques are generally known in the art and are described in detail in methodology treatises such as Advances in Immunology, volume 93, ed. Frederick W. Alt, Academic Press, Burlington, MA, 2007; Making and Using Antibodies: A Practical Handbook, eds. Gary C. Howard and Matthew R. Kaser, CRC Press, Boca Raton, FL, 2006; Medical Immunology, 6^{th} ed., edited by Gabriel Virella, Informa Healthcare Press, London, England, 2007; and Harlow and Lane ANTIBODIES: A Laboratory Manual, Cold Spring Harbor Laboratory Press, Cold Spring Harbor, NY, 1988.

Although compositions and methods similar or equivalent to those described herein can be used in the practice or testing of the present invention, suitable compositions and methods are described below. All publications, patent applications, and patents mentioned herein are incorporated by reference in their entirety. In the case of conflict, the present specification, including definitions, will control. The particular embodiments discussed below are illustrative only and not intended to be limiting.

### Overview

Provided herein are compositions and methods for diagnosing or evaluating a patient suspected of having a brain injury. The method can comprise measuring the level of at least one inflammasome protein in a biological sample obtained from the patient; determining the presence or absence of a protein signature associated with the brain injury, wherein the protein signature comprises an elevated level of the at least one inflammasome protein; and selecting the patient as having the brain injury if the patient exhibits the presence of the protein signature. The brain injury can be any insult to a patient’s brain due to trauma, degeneration or congenital issues. The brain injury can be selected from multiple sclerosis (MS), stroke, Alzheimers Disease (AD), Parkinson’s Disease (PD), cognitive impairment (e.g., mild cognitive impairment (MCI)) or traumatic brain injury (TBI). In one embodiment, the brain injury is MS. In another embodiment, the brain injury is stroke. In yet another embodiment, the brain injury is TBI.. In still another embodiment, the brain injury is MCI.

In one embodiment, provided herein is a method for diagnosing or evaluating a patient of having multiple sclerosis (MS) comprising measuring the level of at least one inflammasome protein in a biological sample obtained from the patient; determining the presence or absence of a protein signature associated with MS, wherein the protein signature comprises an elevated level of the at least one inflammasome protein; and selecting the patient as having the MS if the patient exhibits the presence of the protein signature. The patient can present with clinical symptoms consistent with MS. Through use of the methods and compositions provided herein, the patient can be diagnosed with any type of MS known in the art. The MS can be relapsing-remitting MS (RRMS), secondary-progressive MS (SPMS), primary-progressive MS (PPMS), or progressive-relapsing MS (PRMS).

In another embodiment, provided herein is a method for diagnosing or evaluating a patient suspected of having suffered a stroke, the method comprising: measuring the level of at least one inflammasome protein in a biological sample obtained from the patient; determining the presence or absence of a protein signature associated with stroke or a stroke-related injury, wherein the protein signature comprises an elevated level of the at least one inflammasome protein; and selecting the patient as having suffered from a stroke if the patient exhibits the presence of the protein signature. The patient can present with any clinical symptoms known in the art consistent with stroke. The stroke can be ischemic stroke, transient ischemic stroke or hemorrhagic stroke.

In one embodiment, provided herein is a method for diagnosing or evaluating a patient of having traumatic brain injury (TBI) comprising measuring the level of at least one inflammasome protein in a biological sample obtained from the patient; determining the presence or absence of a protein signature associated with TBI, wherein the protein signature comprises an elevated level of the at least one inflammasome protein; and selecting the patient as having a TBI if the patient exhibits the presence of the protein signature. The patient can present with clinical symptoms consistent with TBI. Through use of the methods and compositions provided herein, the patient can be diagnosed with any type of TBI known in the art.

In one embodiment, provided herein is a method for diagnosing or evaluating a patient of having cognitive impairment. The cognitive impairment can be mild or severe. In one embodiment, the cognitive impairment is mild cognitive impairment (MCI). The method comprises measuring the level of at least one inflammasome protein in a biological sample obtained from the patient; determining the presence or absence of a protein signature associated with cognitive impairment (e.g., MCI), wherein the protein signature comprises an elevated level of the at least one inflammasome protein; and selecting the patient as having a cognitive impairment (e.g., MCI) if the patient exhibits the presence of the protein signature. The patient can present with clinical symptoms consistent with cognitive impairment (e.g., MCI). Through use of the methods and compositions provided herein, the patient can be diagnosed with any type of cognitive impairment known in the art such as, for example, MCI. Examples of symptoms often displayed by subject’s affected with MCI can include forgetfulness (forget things more frequently and/or forget important events), lack of focus (lose train of thought), feel anxious or overwhelmed when making decisions, understanding instructions or planning things, trouble navigating familiar environments, and/or impulsivity and questionable judgment. Subjects with MCI may also experience depression, irritability, anxiety or apathy.

In one aspect of the invention, the method of diagnosing or evaluating a patient suspected of having a brain injury (e.g., MCI, TBI, stroke or MS) comprises determining the presence or absence of a protein signature associated with the brain injury based on the measured level, abundance, or concentration of one or more inflammasome proteins in a biological sample obtained from the patient or on the inflammasome protein profile prepared from a biological sample obtained from the patient. In certain embodiments, the protein signature comprises an elevated level of at least one inflammasome protein. The level of the at least one inflammasome protein in the protein signature may be enhanced relative to the level or percentage of the protein in a biological sample obtained from a control subject or relative to a pre-determined reference value or range of reference values as further described herein. The control subject can be a healthy individual. The healthy individual can be an individual who does not exhibit symptoms associated with the brain injury (e.g., MCI, TBI, stroke or MS). The protein signature may, in certain embodiments, comprise an elevated level at least one inflammasome proteins. Patients who exhibit the protein signature may be selected or identified as having the brain injury (e.g., MCI, TBI, stroke or MS).

In some embodiments, the measured level, concentration, or abundance of one or more inflammasome proteins in the biological sample is used to prepare an inflammasome protein profile, wherein the profile is indicative of the severity of the brain injury (e.g., MCI, TBI, stroke or MS). The inflammasome protein profile may comprise the level, abundance, percentage or concentration of one or more inflammasome proteins measured in the patient’s biological sample optionally in relation to the level, abundance, percentage or concentration of the one or more inflammasome proteins in a biological sample obtained from a control subject or in relation to a pre-determined value or range of reference values as described herein. The control subject can be a healthy individual. The healthy individual can be an individual who does not exhibit symptoms associated with the brain injury (e.g., MCI, TBI, stroke or MS).

The level, percentage or concentration of at least one inflammasome protein can be assessed at a single time point and compared to a pre-determined reference value or range of reference values or can be assessed at multiple time points and compared to a pre-determined reference value or to previously assessed values.

As used herein, “pre-determined reference value” or range of reference values can refer to a pre-determined value or range of reference values of the level or concentration of an inflammasome protein ascertained from a known sample. For instance, the pre-determined reference value or range of reference values can reflect the level or concentration of an inflammasome protein in a biological sample obtained from a control subject (i.e., healthy subject). The control subject may, in some embodiments, be age-matched to the patients being evaluated. The biological sample obtained from the patient and the control subject can both be the same type of sample (e.g., serum or serum-derived extracellular vesicles (EVs). Thus, in particular embodiments, the measured level, percentage or concentration of at least one inflammasome protein is compared or determined relative to the level, percentage or concentration of said at least one inflammasome protein in a control sample (i.e. obtained from a healthy subject). The control or healthy subject can be a subject that does not exhibit symptoms associated with the brain injury (e.g., MCI, TBI, stroke or MS).

In other embodiments, the pre-determined reference value or range of reference values can reflect the level or concentration of an inflammasome protein in a sample obtained from a patient with a known severity of a brain injury (e.g., MCI, TBI, stroke or MS) as assessed by clinical measures or post mortem analysis. A pre-determined reference value can also be a known amount or concentration of an inflammasome protein. Such a known amount or concentration of an inflammasome protein may correlate with an average level or concentration of the inflammasome protein from a population of control subjects or a population of patients with known levels of said brain injury. In another embodiment, the pre-determined reference value can be a range of values, which, for instance, can represent a mean plus or minus a standard deviation or confidence interval. A range of reference values can also refer to individual reference values for a particular inflammasome protein across various levels of brain injury (e.g., MCI, TBI, stroke or MS) severity. In certain embodiments, an increase in the level of one or more inflammasome proteins (e.g., ASC, caspase-1 or IL-18) relative to a pre-determined reference value or range of reference values is indicative of a more severe brain injury.

The at least one inflammasome protein detected or measured in any of the methods provided herein can be one or a plurality of inflammasome proteins. In one embodiment, the at least one inflammasome protein is a plurality of inflammasome proteins. The plurality can be at least or at most 2, 3, 4 or 5 inflammasome proteins. The at least one inflammasome protein or plurality of inflammasome proteins can be a component of any inflammasome known in the art, such as, for example, the NAPL1/NLRP1, NALP2/NLRP2, NALP3/NLRP3, IPAF/NLRC4 or AIM2 inflammasome. In one embodiment, the at least one inflammasome protein is apoptosis-associated speck-like protein containing a caspase recruitment domain (ASC), caspase-1, interleukin-18 (IL-18) or interleukin-1beta (IL-1beta). In one embodiment, the at least one inflammasome protein is apoptosis-associated speck-like protein containing a caspase recruitment domain (ASC). In one embodiment, the at least one inflammasome protein is caspase-1. In one embodiment, the at least one inflammasome protein is IL-18.

The inflammasome proteins of the methods provided herein and other marker proteins can be measured in a biological sample by various methods known to those skilled in the art. For instance, proteins can be measured by methods including, but not limited to, liquid chromatography, gas chromatography, mass spectrometry, immunoassays, radioimmunoassays, immunofluorescent assays, FRET-based assays, immunoblot, ELISAs, or liquid chromatography followed by mass spectrometry (e.g., MALDI MS). One of skill in the art can ascertain other suitable methods for measuring and quantitating any particular biomarker protein of the invention.

In one embodiment, the at least one inflammasome protein or plurality of inflammasome proteins detected or measured in any of the methods provided herein can be detected or measured through the use of an immunoassay. The immunoassay can be any immunoassay known in the art. For example, the immunoassay can be an immunoblot, enzyme-linked immunosorbent assay (ELISA) or a microfluidic immunoassay. An example of a microfluidic immunoassay for use in the methods provided herein is the Simple Plex™ Platform (Protein Simple, San Jose, California).

Any immunoassay for use in the methods provided herein can utilize an antibody directed against an inflammasome protein. The inflammasome component can be a component of any inflammasome known in the art, such as, for example, the NAPL1, NALP2, NALP3, NLRC4 or AIM2 inflammasome. In one embodiment, the inflammasome protein is apoptosis-associated speck-like protein containing a caspase recruitment domain (ASC), caspase-1, interleukin-18 (IL-18) or interleukin-1beta (IL-1beta). In one embodiment, the inflammasome protein is apoptosis-associated speck-like protein containing a caspase recruitment domain (ASC). In one embodiment, the inflammasome protein is caspase-1. In one embodiment, the inflammasome protein is IL-18. In one embodiment, the inflammasome protein is IL-1beta.

Any suitable antibody that specifically binds ASC can be used, e.g., a custom or commercially available ASC antibody can be used in the methods provided herein. The anti-ASC antibody can be an antibody that specifically binds to a domain or portion thereof of a mammalian ASC protein such as, for example a human or rat ASC protein. Examples of anti-ASC antibodies for use in the methods herein can be those found in US8685400, the contents of which are herein incorporated by reference in its entirety. Examples of commercially available anti-ASC antibodies for use in the methods provided herein include, but are not limited to 04-147 Anti-ASC, clone 2EI-7 mouse monoclonal antibody from MilliporeSigma, AB3607 - Anti-ASC Antibody from Millipore Sigma, orb194021 Anti-ASC from Biorbyt, LS-C331318-50 Anti-ASC from LifeSpan Biosciences, AF3805 Anti-ASC from R & D Systems, NBP1-78977 Anti-ASC from Novus Biologicals, 600-401-Y67 Anti-ASC from Rockland Immunochemicals, D086-3 Anti-ASC from MBL International, AL177 anti-ASC from Adipogen, monoclonal anti-ASC (clone o93E9) antibody, anti-ASC antibody (F-9) from Santa Cruz Biotechnology, anti-ASC antibody (B-3) from Santa Cruz Biotechnology, ASC polyclonal antibody - ADI-905-173 from Enzo Life Sciences, or A161 Anti-Human ASC - Leinco Technologies. The human ASC protein can be accession number NP_037390.2 (Q9ULZ3-1), NP_660183 (Q9ULZ3-2) or Q9ULZ3-3. The rat ASC protein can be accession number NP_758825 (BAC43754). The mouse ASC protein can be accession number NP_075747.3. In one embodiment, the antibody binds to a PYRIN-PAAD-DAPIN domain (PYD) or a portion or fragment thereof of a mammalian ASC protein (e.g. human or rat ASC). In this embodiment, an antibody as described herein specifically binds to an amino acid sequence having at least 65% (e.g., 65, 70, 75, 80, 85%) sequence identity with a PYD domain or fragment thereof of human or rat ASC. In one embodiment, the antibody binds to a C-terminal caspase-recruitment domain (CARD) or a portion or fragment thereof of a mammalian ASC protein (e.g. human or rat ASC). In this embodiment, an antibody as described herein specifically binds to an amino acid sequence having at least 65% (e.g., 65, 70, 75, 80, 85%) sequence identity with a CARD domain or fragment thereof of human or rat ASC. In another embodiment, the antibody is an antibody that specifically binds to a region of rat ASC, e.g., amino acid sequence ALRQTQPYLVTDLEQS (SEQ ID NO: 1) (i.e., residues 178-193 of rat ASC, accession number BAC43754). In this embodiment, an antibody as described herein specifically binds to an amino acid sequence having at least 65% (e.g., 65, 70, 75, 80, 85%) sequence identity with amino acid sequence ALRQTQPYLVTDLEQS (SEQ ID NO: 1) of rat ASC. In another embodiment, the antibody is an antibody that specifically binds to a region of human ASC, e.g., amino acid sequence RESQSYLVEDLERS (SEQ ID NO: 2). In this embodiment, an antibody as described herein specifically binds to an amino acid sequence having at least 65% (e.g., 65, 70, 75, 80, 85%) sequence identity with amino acid sequence RESQSYLVEDLERS (SEQ ID NO: 2) of human ASC.

Any suitable anti-NLRP1 antibody (e.g., commercially available or custom) can be used in the methods provided herein. Examples of anti-NLRP1 antibodies for use in the methods herein can be those found in US8685400, the contents of which are herein incorporated by reference in its entirety. Examples of commercially available anti-NLRP1 antibodies for use in the methods provided herein include, but are not limited to human NLRP1 polyclonal antibody AF6788 from R&D Systems, EMD Millipore rabbit polyclonal anti-NLRP1 ABF22, Novus Biologicals rabbit polyclonal anti-NLRP1 NB100-56148, Sigma-Aldrich mouse polyclonal anti-NLRP1 SAB1407151, Abcam rabbit polyclonal anti-NLRP1 ab3683, Biorbyt rabbit polyclonal anti-NLRP1 orb325922 mybiosource rabbit polyclonal anti-NLRP1 MBS7001225, R&D systems sheep polyclonal AF6788, Aviva Systems mouse monoclonal anti-NLRP1 oaed00344, Aviva Systems rabbit polyclonal anti-NLRP1 ARO54478_P050, Origene rabbit polyclonal anti-NLRP1 APO7775PU-N, Antibodies online rabbit polyclonal anti-NLRP1 ABIN768983, Prosci rabbit polyclonal anti-NLRP1 3037, Proteintech rabbit polyclonal anti-NLRP1 12256-1-AP, Enzo mouse monoclonal anti-NLRP1 ALX-804-803-C100, Invitrogen mouse monoclonal anti-NLRP1 MA1-25842, GeneTex mouse monoclonal anti-NLRP1 GTX16091, Rockland rabbit polyclonal anti-NLRP1 200-401-CX5, or Cell Signaling Technology rabbit polyclonal anti-NLRP1 4990. The human NLRP1 protein can be accession number AAH51787, NP_001028225, NP_055737, NP_127497, NP_127499, or NP_127500. In one embodiment, the antibody binds to a Pyrin, NACHT, LRR1-6, FIIND or CARD domain or a portion or fragment thereof of a mammalian NLRP1 protein (e.g. human NLRP1). In this embodiment, an antibody as described herein specifically binds to an amino acid sequence having at least 65% (e.g., 65, 70, 75, 80, 85%) sequence identity with a specific domain (e.g., Pyrin, NACHT, LRR1-6, FIIND or CARD) or fragment thereof of human NLRP1. In one embodiment, a chicken anti-NLRP1 polyclonal that was custom-designed and produced by Ayes Laboratories can be used. This antibody can be directed against the following amino acid sequence in human NLRP1: CEYYTEIREREREKSEKGR (SEQ ID NO: 3). In one embodiment, the antibody specifically binds to an amino acid sequence having at least 85% sequence identity with amino acid sequence SEQ ID NO: 3 or SEQ ID NO: 4.

Any suitable antibody that specifically binds caspase-1 can be used, e.g., a custom or commercially available, in the methods provided herein. Examples of commercially available anti-caspase-1 antibodies for use in the methods provided herein include: R&D Systems: Cat# MAB6215, or Cat#AF6215; Cell Signaling: Cat #3866, #225, or #4199; Novus Biologicals: Cat #NB100-56565, #NBPI-45433, #NB100-56564, #MAB6215, #AF6215, #NBP2-67487, #NBP2-15713, #NBP2-15712, #NBP1-87680, #NB120-1872, #NBP1-76605, or # H00000834-M01.

Any suitable antibody that specifically binds IL-18 can be used, e.g., a custom or commercially available, in the methods provided herein. Examples of commercially available anti-IL-18 antibodies for use in the methods provided herein include: R&D Systems: Cat# D044-3, Cat# D045-3, #MAB646, #AF2548, #D043-3, # MAB2548, MAB9124, # MAB91241, # MAB91243, MAB91244, or # MAB91242; Novus Biologicals: Cat #AF2548, # D043-3, # MAB2548, # MAB9124, # MAB91243, # MAB91244, # MAB91241, # D045-3, # MAB91242, or #D044-3.

Any suitable antibody that specifically binds IL-1beta can be used, e.g., a custom or commercially available, in the methods provided herein. Examples of commercially available anti-IL-18 antibodies for use in the methods provided herein include: R&D Systems: Cat# MAB601, Cat# MAB201, # MAB6964, # MAB601R, #MAB8406, or # MAB6215; Cell Signaling: Cat #31202, #63124, #12426, or #12507; Novus Biologicals: Cat #AF-201-NA, #NB600-633, #MAB201, #MAB601, #NBP1-19775, #NBP2-27345, #AB-201-NA, #NBP2-27342, #NBP2-67865, #NBP2-27343, #NBP2-27340, #NBP2-27340, #NB120-8319, #23600002, #MAB8406, #NB100-73053, #NB120-10749, or # MAB601R.

Methods for determining monoclonal antibody specificity and affinity by competitive inhibition can be found in Harlow, et al., Antibodies: A Laboratory Manual, Cold Spring Harbor Laboratory Press, Cold Spring Harbor, N.Y., 1988, Colligan et al., eds., Current Protocols in Immunology, Greene Publishing Assoc. and Wiley Interscience, N.Y., (1992, 1993), and Muller, Meth. Enzymol. 92:589-601, 1983, which references are entirely incorporated herein by reference.

Anti-inflammasome (e.g., Anti-ASC and anti-NLRP1) antibodies of the present invention can be routinely made according to methods such as, but not limited to inoculation of an appropriate animal with the polypeptide or an antigenic fragment, in vitro stimulation of lymphocyte populations, synthetic methods, hybridomas, and/or recombinant cells expressing nucleic acid encoding such anti-ASC or anti-NLRP1 antibodies. Immunization of an animal using purified recombinant ASC or peptide fragments thereof, e.g., residues 178-193 (SEQ ID NO: 1) of rat ASC (e.g., accession number BAC43754) or SEQ ID NO: 2 of human ASC, is an example of a method of preparing anti-ASC antibodies. Similarly, immunization of an animal using purified recombinant NLRP1 or peptide fragments thereof, e.g., residues MEE SQS KEE SNT EG-cys (SEQ ID NO: 4) of rat NALP1 or SEQ ID NO: 3 of human NALP1, is an example of a method of preparing anti-NLRP1 antibodies.

Monoclonal antibodies that specifically bind ASC or NLRP1 may be obtained by methods known to those skilled in the art. See, for example Kohler and Milstein, Nature 256:495-497, 1975; U.S. Pat. No. 4,376,110; Ausubel et al., eds., Current Protocols in Molecular Biology, Greene Publishing Assoc. and Wiley Interscience, N.Y., (1987, 1992); Harlow and Lane ANTIBODIES: A Laboratory Manual Cold Spring Harbor Laboratory Press, Cold Spring Harbor, NY, 1988; Colligan et al., eds., Current Protocols in Immunology, Greene Publishing Assoc. and Wiley Interscience, N.Y., (1992, 1993), the contents of which are incorporated entirely herein by reference. Such antibodies may be of any immunoglobulin class including IgG, IgM, IgE, IgA, GILD and any subclass thereof A hybridoma producing a monoclonal antibody of the present invention may be cultivated in vitro, in situ or in vivo.

In any of the methods provided herein, the “biological sample” can refer to any bodily fluid or tissue obtained from a patient or subject. A biological sample can include, but is not limited to, whole blood, red blood cells, plasma, serum, peripheral blood mononuclear cells (PBMCs), urine, saliva, tears, buccal swabs, CSF, CNS microdialysate, and nerve tissue. In one embodiment, the biological sample is CSF, saliva, serum, plasma, or urine. In certain embodiments, the biological sample is CSF. In another embodiment, the biological sample is serum-derived extracellular vesicles (EVs). The EVs can be isolated from serum by any method known in the art. It should be noted that a biological sample obtained from a patient or test subject can be of the same type as a biological sample obtained from a control subject.

In some instances, the methods provided herein can be capable of diagnosing or detecting a brain injury (e.g., MCI, stroke, MS or TBI) with a predictive success of at least about 70%, at least about 71%, at least about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, up to 100%.

In some instances, the methods provided herein can be capable of diagnosing or detecting a brain injury (e.g., MCI, stroke, MS or TBI) with a sensitivity and/or specificity of at least about 70%, at least about 71%, at least about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, up to 100%.

In one embodiment, the brain injury is MS such that detection of an elevated level of ASC in serum obtained from the patient as compared to a control (e.g., a pre-determined reference value or range of reference values) as provided herein determines that the patient has MS with a sensitivity of at least 75, 80, 90%, 95%, 99% or 100%. In another embodiment, the brain injury is MS such that detection of an elevated level of ASC in serum obtained from the patient as compared to a control (e.g., a pre-determined reference value or range of reference values) as provided herein determines that the patient has MS with a specificity of at least 75%, 80%, 85%, 90%, 95%, 99% or 100%. The pre-determined reference value for these embodiments can be the cut-off values shown in Table 7. In yet another embodiment, the brain injury is MS such that detection of an elevated level of ASC in serum obtained from the patient as compared to a control (e.g., a pre-determined reference value or range of reference values) as provided herein determines that the patient has MS with a sensitivity of at least 90%, and a specificity of at least 80%. The pre-determined reference value for this embodiment can be the cut-off values shown in Table 7. In some cases, the range of reference values can be from about 300 pg/ml to about 340 pg/ml to attain a sensitivity of at least 90% and a specificity of at least 80%.

In one embodiment, the brain injury is stroke such that detection of an elevated level of ASC in serum obtained from the patient as compared to a control (e.g., a pre-determined reference value or range of reference values) as provided herein determines that the patient has suffered a stroke with a sensitivity of at least 75, 80, 90%, 95%, 99% or 100%. In another embodiment, the brain injury is stroke such that detection of an elevated level of ASC in serum obtained from the patient as compared to a control (e.g., a pre-determined reference value or range of reference values) as provided herein determines that the patient has MS with a specificity of at least 75%, 80%, 85%, 90%, 95%, 99% or 100%. The pre-determined reference value for these embodiments can be the cut-off values shown in Table 8. In another embodiment, the brain injury is stroke such that detection of an elevated level of ASC in serum obtained from the patient as compared to a control (e.g., a pre-determined reference value or range of reference values) as provided herein determines that the patient suffered a stroke with a sensitivity of at least 100% and a specificity of at least 90%. The pre-determined reference value for this embodiment can be the cut-off values shown in Table 8. In some cases, the range of reference values can be from about 380 pg/ml to about 405 pg/ml to attain a sensitivity of at least 100% and a specificity of at least 90%. The stroke can be ischemic or hemorraghic as provided herein.

In one embodiment, the brain injury is stroke such that detection of an elevated level of ASC in serum-derived EVs obtained from the patient as compared to a control (e.g., a pre-determined reference value or range of reference values) as provided herein determines that the patient has suffered a stroke with a sensitivity of at least 75%, 80%, 85%, 90%, 95%, 99% or 100%. In another embodiment, the brain injury is stroke such that detection of an elevated level of ASC in serum-derived EVs obtained from the patient as compared to a control (e.g., a pre-determined reference value or range of reference values) as provided herein determines that the patient has MS with a specificity of at least 75, 80, 90%, 95%, 99% or 100%. The pre-determined reference value for these embodiments can be the cut-off values shown in Table 9. In another embodiment, the brain injury is stroke such that detection of an elevated level of ASC in serum-derived EVs obtained from the patient as compared to a control (e.g., a pre-determined reference value or range of reference values) as provided herein determines that the patient suffered a stroke with a sensitivity of at least 100% and a specificity of at least 90%. The pre-determined reference value for this embodiment can be the cut-off values shown in Table 9. In some cases, the range of reference values can be from about 70 pg/ml to about 90 pg/ml to attain a sensivity of at least 100% and a specificity of at least 90%. The stroke can be ischemic or hemorraghic as provided herein.

In one embodiment, the brain injury is TBI such that detection of an elevated level of ASC in serum obtained from the patient as compared to a control (e.g., a pre-determined reference value or range of reference values) as provided herein determines that the patient has TBI with a sensitivity of at least 75, 80, 90%, 95%, 99% or 100%. In another embodiment, the brain injury is TBI such that detection of an elevated level of ASC in serum obtained from the patient as compared to a control (e.g., a pre-determined reference value or range of reference values) as provided herein determines that the patient has TBI with a specificity of at least 75%, 80%, 85%, 90%, 95%, 99% or 100%. The pre-determined reference value for these embodiments can be the cut-off values shown in Table 16. In yet another embodiment, the brain injury is TBI such that detection of an elevated level of ASC in serum obtained from the patient as compared to a control (e.g., a pre-determined reference value or range of reference values) as provided herein determines that the patient has TBI with a sensitivity of at least 90%, and a specificity of at least 80%. The pre-determined reference value for this embodiment can be the cut-off values shown in Table 16. In some cases, the range of reference values can be from about 275 pg/ml to about 450 pg/ml to attain a sensitivity of at least 80% and a specificity of at least 70%.

In one embodiment, the brain injury is TBI such that detection of an elevated level of caspase-1 in serum obtained from the patient as compared to a control (e.g., a pre-determined reference value or range of reference values) as provided herein determines that the patient has TBI with a sensitivity of at least 75, 80, 90%, 95%, 99% or 100%. In another embodiment, the brain injury is TBI such that detection of an elevated level of caspase-1 in serum obtained from the patient as compared to a control (e.g., a pre-determined reference value or range of reference values) as provided herein determines that the patient has TBI with a specificity of at least 75%, 80%, 85%, 90%, 95%, 99% or 100%. The pre-determined reference value for these embodiments can be the cut-off values shown in Table 15. In yet another embodiment, the brain injury is TBI such that detection of an elevated level of caspase-1 in serum obtained from the patient as compared to a control (e.g., a pre-determined reference value or range of reference values) as provided herein determines that the patient has TBI with a sensitivity of at least 90%, and a specificity of at least 80%. The pre-determined reference value for this embodiment can be the cut-off values shown in Table 15. In some cases, the range of reference values can be from about 2.812 pg/ml to about 1.853 pg/ml to attain a sensitivity of at least 70% and a specificity of at least 75%.

In one embodiment, the brain injury is MCI such that detection of an elevated level of ASC in serum obtained from the patient as compared to a control (e.g., a pre-determined reference value or range of reference values) as provided herein determines that the patient has MCI with a sensitivity of at least 75%, 80%, 85%, 90%, 95%, 99% or 100%. In another embodiment, the brain injury is MCI such that detection of an elevated level of ASC in serum obtained from the patient as compared to a control (e.g., a pre-determined reference value or range of reference values) as provided herein determines that the patient has MCI with a specificity of at least 50%, 55%, 60% 65%, 70%, 75%, 80%, 85%, 90%, 95%, 99% or 100%. The pre-determined reference value for these embodiments can be the cut-off values shown in Tables 22 and 23. In yet another embodiment, the brain injury is MCI such that detection of an elevated level of ASC in serum obtained from the patient as compared to a control (e.g., a pre-determined reference value or range of reference values) as provided herein determines that the patient has MCI with a sensitivity of at least 90%, and a specificity of at least 70%. The pre-determined reference value(s) for this embodiment can be the cut-off values shown in Tables 22 and 23. In some cases, the range of reference values can be about 257 pg/ml to about 342 pg/ml to attain a sensitivity of at least 90% and a specificity of at least 70%.

In one embodiment, the brain injury is MCI such that detection of an elevated level of IL-18 in serum obtained from the patient as compared to a control (e.g., a pre-determined reference value or range of reference values) as provided herein determines that the patient has MCI with a sensitivity of at least 75%, 80%, 85%, 90%, 95%, 99% or 100%. In another embodiment, the brain injury is MCI such that detection of an elevated level of IL-18 in serum obtained from the patient as compared to a control (e.g., a pre-determined reference value or range of reference values) as provided herein determines that the patient has MCI with a specificity of at least 50%, 55%, 60%, 65%, 75%, 80%, 85%, 90%, 95%, 99% or 100%. The pre-determined reference value for these embodiments can be the cut-off values shown in Tables 22 and 25. In yet another embodiment, the brain injury is MCI such that detection of an elevated level of IL-18 in serum obtained from the patient as compared to a control (e.g., a pre-determined reference value or range of reference values) as provided herein determines that the patient has MCI with a sensitivity of at least 70%, and a specificity of at least 55%. The pre-determined reference value for this embodiment can be the cut-off values shown in Tables 22 and 25. In some cases, the range of reference values from about 200 pg/ml to about 214 pg/ml to attain a sensitivity of at least 70% and a specificity of at least 50%.

In any of the methods provided herein, the sensitivity and/or specificity of an inflammasome protein (e.g., ASC) for predicting or diagnosing a brain injury (e.g., MCI, stroke, MS or TBI) is determined by calculation of area under curve (AUC) values with confidence intervals (e.g., 95%). The area under curve (AUC) can be determined from receiver operator characteristic (ROC) curves with confidence intervals of 95%.

In one embodiment, the brain injury is MS such that detection of a level or concentration of at least one inflammasome protein in a biological sample obtained from the patient that is elevated by a pre-determined percentage over the level of the same at least one inflammasome protein in a biological sample obtained from a control subject is indicative of the patient as having MS. The biological sample obtained from the patient and the control subject can be of the same type (e.g., serum or serum-derived EVs). The pre-determined percentage can be about, at most or at least 50%, 55%, 60%, 65%, 70%, 75%, 80%, 85%, 90%, 95%, 99% 100%, 110%, 120%, 130%, 140% 150%, 160%, 170%, 180%, 190% or 200%. The at least one inflammasome protein can be selected from caspase-1, IL-18, IL-1beta and ASC. In one embodiment, the brain injury is MS such that detection of a level or concentration of ASC in serum obtained from the patient that is at least 50% higher than the level of ASC in a serum sample obtained from a control subject is indicative of the patient as having MS.

In one embodiment, the brain injury is stroke such that detection of a level or concentration of at least one inflammasome protein in a biological sample obtained from the patient that is elevated by a pre-determined percentage over the level of the same at least one inflammasome protein in a biological sample obtained from a control subject is indicative of the patient as having MS. The biological sample obtained from the patient and the control subject can be of the same type (e.g., serum or serum-derived EVs). The pre-determined percentage can be about, at most or at least 50%, 55%, 60%, 65%, 70%, 75%, 80%, 85%, 90%, 95%, 99% 100%, 110%, 120%, 130%, 140% 150%, 160%, 170%, 180%, 190% or 200%. The at least one inflammasome protein can be selected from caspase-1, IL-18, IL-1beta and ASC. In one embodiment, the brain injury is stroke such that detection of a level or concentration of ASC in serum obtained from the patient that is at least 70% higher than the level of ASC in a serum sample obtained from a control subject is indicative of the patient as having suffered a stroke. In one embodiment, the brain injury is stroke such that detection of a level or concentration of ASC in serum-derived EVs obtained from the patient that is at least 110% higher than the level of ASC in a serum-derived EVs sample obtained from a control subject is indicative of the patient as having suffered a stroke.

In one embodiment, the brain injury is TBI such that detection of a level or concentration of at least one inflammasome protein in a biological sample obtained from the patient that is elevated by a pre-determined percentage over the level of the same at least one inflammasome protein in a biological sample obtained from a control subject is indicative of the patient as having TBI. The biological sample obtained from the patient and the control subject can be of the same type (e.g., serum or serum-derived EVs). The pre-determined percentage can be about, at most or at least 50%, 55%, 60%, 65%, 70%, 75%, 80%, 85%, 90%, 95%, 99% 100%, 110%, 120%, 130%, 140% 150%, 160%, 170%, 180%, 190% or 200%. The at least one inflammasome protein can be selected from caspase-1, IL-18, IL-1beta and ASC. In one embodiment, the brain injury is TBI such that detection of a level or concentration of ASC in serum obtained from the patient that is at least 50% higher than the level of ASC in a serum sample obtained from a control subject is indicative of the patient as having TBI.

In one embodiment, the brain injury is MCI such that detection of a level or concentration of at least one inflammasome protein in a biological sample obtained from the patient that is elevated by a pre-determined percentage over the level of the same at least one inflammasome protein in a biological sample obtained from a control subject is indicative of the patient as having MCI. The biological sample obtained from the patient and the control subject can be of the same type (e.g., serum or serum-derived EVs). The pre-determined percentage can be about, at most or at least 50%, 55%, 60%, 65%, 70%, 75%, 80%, 85%, 90%, 95%, 99% 100%, 110%, 120%, 130%, 140% 150%, 160%, 170%, 180%, 190% or 200%. The at least one inflammasome protein can be selected from caspase-1, IL-18, IL-1beta and ASC. In one embodiment, the brain injury is MCI such that detection of a level or concentration of ASC in serum obtained from the patient that is at least 50% higher than the level of ASC in a serum sample obtained from a control subject is indicative of the patient as having MCI.

The present invention also provides a method of determining a prognosis for a patient with a brain injury (e.g., MCI, stroke, MS or TBI). In one embodiment, the method comprises providing a biological sample obtained from the patient and measuring the level of at least one inflammasome protein in the biological sample to prepare an inflammasome protein profile as described above, wherein the inflammasome protein profile is indicative of the prognosis of the patient. In some embodiments, an increase in the level of one or more inflammasome proteins (e.g., IL-18, NLRP1, ASC, caspase-1, or combinations thereof) relative to a pre-determined reference value or range of reference values is indicative of a poorer prognosis. For instance, an increase of about 20% to about 300% in the level of one or more inflammasome proteins relative to a pre-determined reference value or range of reference values is indicative of a poorer prognosis. In some cases, the inflammasome protein is ASC and the pre-determined reference values can be derived from Tables 7-9, 16, 22 or 23.

### Methods of Treatment

In other embodiments of the invention, the methods of diagnosing or evaluating a patient as having a brain injury (e.g., MCI, stroke, MS or TBI) further comprises administering a standard of care treatment for said brain injury (e.g., MCI, TBI, stroke or MS) to the patient based on the measured level of said at least one inflammasome protein or when a protein signature associated with a brain injury (e.g., MCI, stroke or MS or TBI) is identified. The methods of diagnosing or evaluating a patient as having a brain injury (e.g., MCI, stroke, MS or TBI) can be ascertained using the methods described herein. In some embodiment, the methods of diagnosing or evaluating a patient having a brain injury further comprises administering a neuroprotective treatment to the patient based on the measured level of said at least one inflammasome protein or when a protein signature associated with a brain injury or a more severe brain injury is identified. Such neuroprotective treatments include drugs that reduce excitotoxicity, oxidative stress, and inflammation. Thus, suitable neuroprotective treatments include, but are not limited to, methylprednisolone, 17alpha-estradiol, 17beta-estradiol, ginsenoside, progesterone, simvastatin, deprenyl, minocycline, resveratrol, and other glutamate receptor antagonists (e.g. NMDA receptor antagonists) and antioxidants. In some embodiments, neuroprotective treatments are antibodies against an inflammasome protein or binding fragments thereof, such as the antibodies directed against inflammasome proteins provided herein.

The success of, or response to, a standard of care treatment can also be monitored by measuring the levels of at least one inflammasome protein. Accordingly, in some embodiments, the methods of evaluating or diagnosing a patient with a brain injury (e.g., MCI, stroke, MS or TBI) further comprise measuring the level of at least one inflammasome protein in a biological sample obtained from the patient following treatment, preparing a treatment protein signature associated with a positive response to the treatment, wherein the treatment protein signature comprises a reduced level of at least one inflammasome protein, and identifying patients exhibiting the presence of the treatment protein signature as responding positively to the treatment. A reduction in the level, abundance, or concentration of one or more inflammasome proteins (e.g. ASC, IL-18 or caspase-1) is indicative of the efficacy of the treatment in the patient. The one or more inflammasome proteins measured in the sample obtained following treatment may be the same as or different than the inflammasome proteins measured in the sample obtained prior to treatment. The inflammasome protein levels may also be used to adjust dosage or frequency of a treatment. The inflammasome protein levels can be ascertained using the methods and techniques provided herein.

In one embodiment, the brain injury (e.g., MCI, TBI, stroke or MS) is MS and the standard of care treatment is selected from is selected from therapies directed towards modifying disease outcome, managing relapses, managing symptoms or any combination thereof. The therapies directed toward modifying disease outcome can be selected from beta-interferons, glatiramer acetate, fingolimod, teriflunomide, dimethyl fumarate, mitoxanthrone, ocrelizumab, alemtuzumab, daclizumab and natalizumab. wherein the stroke is ischemic stroke, transient ischemic stroke or hemorrhagic stroke.

In another embodiment, the brain injury (e.g., MCI, TBI, stroke or MS) is ischemic stroke or transient ischemic stroke and the standard of care treatment is selected from tissue plasminogen activator (tPA), antiplatelet medicine, anticoagulants, a carotid artery angioplasty, carotid endarterectomy, intra-arterial thrombolysis and mechanical clot removal in cerebral ischemia (MERCI) or a combination thereof. In still another embodiment, the brain injury (e.g., TBI, stroke or MS) is hemorrhagic stroke and the standard of care treatment is an aneurysm clipping, coil embolization or arteriovenous malformation (AVM) repair.

In another embodiment, the brain injury (e.g., MCI, TBI, stroke or MS) is TBI and the standard of care treatment is selected from diuretics, anti-seizure drugs, coma inducing drugs, surgery and/or rehabilitation. Diuretics can be used to reduce the amount of fluid in tissues and increase urine output. Diuretics, given intravenously to people with traumatic brain injury, can help reduce pressure inside the brain. An anti-seizure drug may be given during the first week to avoid any additional brain damage that might be caused by a seizure. Continued anti-seizure treatments are used only if seizures occur. Coma-inducing drugs can sometimes be used drugs to put people into temporary comas because a comatose brain needs less oxygen to function. This can be especially helpful if blood vessels, compressed by increased pressure in the brain, are unable to supply brain cells with normal amounts of nutrients and oxygen. The severity of the TBI can be assessed using the Glasgow Coma Scale. This 15-point test can help a doctor or other emergency medical personnel assess the initial severity of a brain injury by checking a person’s ability to follow directions and move their eyes and limbs. The coherence of speech can also provides important clues. Abilities are scored from three to 15 in the Glasgow Coma Scale. Higher scores mean less severe injuries.

In yet another embodiment, the brain injury (e.g., MCI, TBI, stroke or MS) is MCI and the standard of care treatment is selected from computerized cognitive training, group memory training, individual errorless learning sessions, family memory strategy interventions, DHA (docosahexaenoic acid), EPA (eicosapentanoic acid), ginko biloba, donepezil, rivastigimine, triflusal, Huannao Yicong capsules, piribedil, nicotine patch, vitamin E, vitamins B12 & B6, folic acid, rofecoxib, galantamine, cholinesterase inhibitors memantine, lithium, Wuzi Yanzong granules, ginseng, and exercise.

### Kits

Also provided herein are kits for preparing an inflammasome protein profile associated with a brain injury (e.g., MCI, stroke, MS or TBI). The kits may include a reagent for measuring at least one inflammasome protein and instructions for measuring said at least one inflammasome protein for assessing the severity of a brain injury (e.g., MCI, stroke, MS or TBI) in a patient. As used herein, a “reagent” refers to the components necessary for detecting or quantitating one or more proteins by any one of the methods described herein. For instance, in some embodiments, kits for measuring one or more inflammasome proteins can include reagents for performing liquid or gas chromatography, mass spectrometry, immunoassays, immunoblots, or electrophoresis to detect one or more inflammasome proteins as described herein. In some embodiments, the kit includes reagents for measuring one or more inflammasome proteins selected from IL-18, ASC, caspase-1, or combinations thereof.

In one embodiment, the kit comprises a labeled-binding partner that specifically binds to one or more inflammasome proteins, wherein said one or more inflammasome proteins are selected from the group consisting of IL-18, ASC, caspase-1, and combinations thereof. Suitable binding partners for specifically binding to inflammasome proteins include, but are not limited to, antibodies and fragments thereof, aptamers, peptides, and the like. In certain embodiments, the binding partners for detecting ASC are antibodies or fragments thereof, The antibodies directed to ASC can be any antibodies known in the art and/or commercially available. Examples of anti-ASC antibodies for use in the methods provided herein are described herein. In certain embodiments, the binding partners for detecting ASC are antibodies or fragments thereof, aptamers, or peptides that specifically bind to the amino acid sequence of SEQ ID NO: 1 or SEQ ID NO: 2 of rat ASC and human ASC, respectively. In certain embodiments, the binding partners for detecting IL-18 are antibodies or fragments thereof. The antibodies to IL-18 can be any antibodies known in the art and/or commercially available, such as those, for example, provided herein. In certain embodiments, the binding partners for detecting caspase-1 are antibodies or fragments thereof. The antibodies to caspase-1 can be any antibodies known in the art and/or commercially available, such as those, for example, provided herein. In certain embodiments, the binding partners for detecting IL-1beta are antibodies or fragments thereof. The antibodies to IL-1beta can be any antibodies known in the art and/or commercially available, such as those, for example, provided herein. Labels that can be conjugated to the binding partner include metal nanoparticles (e.g., gold, silver, copper, platinum, cadmium, and composite nanoparticles), fluorescent labels (e.g., fluorescein, Texas-Red, green fluorescent protein, yellow fluorescent protein, cyan fluorescent protein, Alexa dye molecules, etc.), and enzyme labels (e.g., alkaline phosphatase, horseradish peroxidase, beta-galactosidase, beta-lactamase, galactose oxidase, lactoperoxidase, luciferase, myeloperoxidase, and amylase).

### EXAMPLES

The present invention is further illustrated by the following specific examples. The examples are provided for illustration only and should not be construed as limiting the scope of the invention in any way.

### Example 1: Examination of Inflammasome Proteins as Biomarkers of Multiple Sclerosis (MS)

Multiple sclerosis (MS) is an autoimmune disease that affects the brain and spinal cord. Important to the care of patients with MS is the need for biomarkers that can predict disease onset, disease exacerbation as well as response to treatment¹.

The inflammasome is a key mediator of the innate immune response that in the CNS was first described to mediate inflammation after spinal cord injury². The inflammasome is a multiprotein complex involved in the activation of caspase-1 and the processing of the pro-inflammatory cytokines IL-1β and IL-18 ³.

In this example, the expression level of inflammasome proteins in serum samples from patients with MS are determined. Further, an examination of the sensitivity and specificity of inflammasome signaling proteins as biomarkers of MS was examined.

### Materials and Methods

### Participants

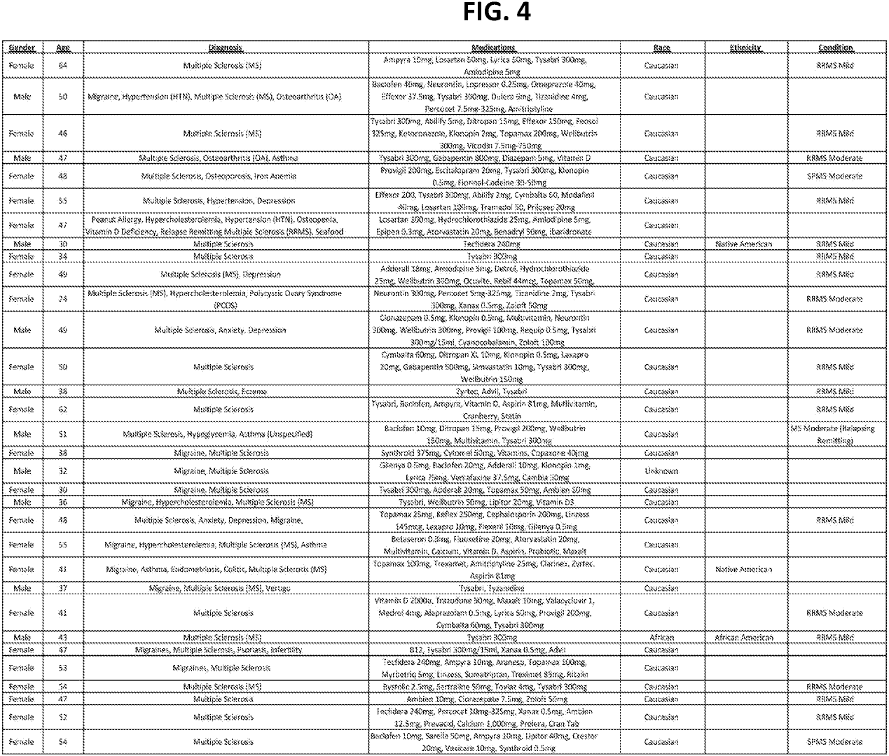

In this study, serum samples were analyzed from 120 normal donors and 32 patients that were diagnosed with MS. Samples were purchased from BioreclamationIVT. The normal donor group consisted of samples obtained from 60 male and 60 female donors in the age range of 20 to 70 years old. The age range in the MS group consisted of samples obtained from patients in the age range of 24 to 64 years old (FIG. 4).

### Protein Assay

Concentration of inflammasome proteins ASC, IL-1β and IL-18 in serum was analyzed using a Simple Plex and a Simple Plex Explorer software. Results shown correspond to the mean of each sample run in triplicates. It should be noted that any system/instrument known in the art can be used to measure the levels of proteins (e.g., inflammasome proteins) in bodily fluids.

### Biomarker Analyses

Prism 7 software (GraphPad) was used to analyze the data obtained from the Simple Plex Explorer Software. Comparisons between groups were carried after identifying outliers followed by determination of the area under the receiver operator characteristic (ROC) curve, as well as the 95% confidence interval (CI). The p-value of significance used was <0.05. Sensitivity and specificity of each biomarker was obtained for a range of different cut-off points. Samples that yielded a protein value below the level of detection of the assay were not included in the analyses for that analyte.

ROC curves are summarized as the area under the curve (AUC). A perfect AUC value is 1.0, where 100% of subjects in the population will be correctly classified as having MS or not. In contrast, an AUC of 0.5 signifies that subjects are randomly classified as either positive or negative for MS, which has no clinical utility. It has been suggested that an AUC between 0.9 to 1.0 applies to an excellent biomarker; from 0.8 to 0.9, good; 0.7 to 0.8 fair; 0.6 to 0.7, poor and 0.5 to 0.6, fail. ¹⁰

### Results

### Caspase-1, ASC and IL-18 are Elevated in the Serum of MS Patients

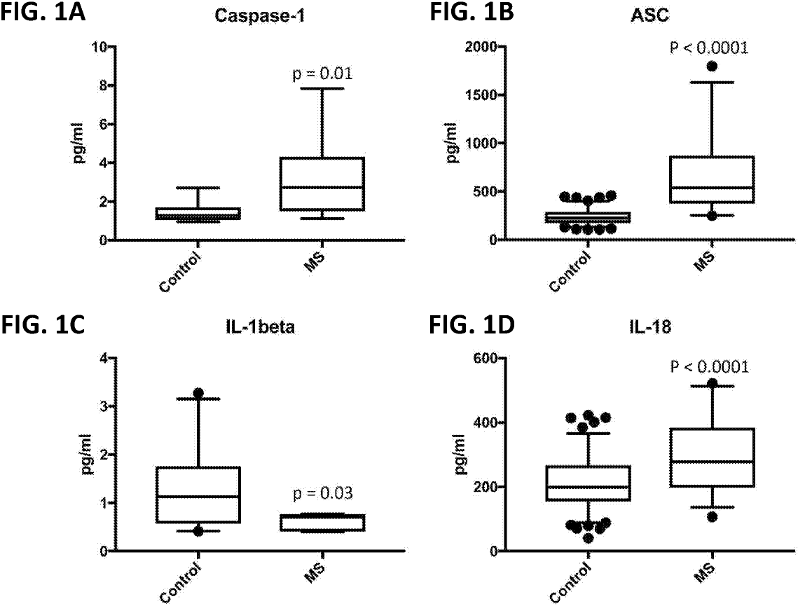

Serum samples from MS patients were analyzed and compared to serum from healthy/control individuals using a Simple Plex assay (Protein Simple) for the protein expression of the inflammasome signaling proteins caspase-1, ASC, IL-1β and IL-18 (FIGS. 1A-1D). The protein levels of caspase-1, ASC and IL-18 in the serum of MS patients was higher than in the control group. However, the levels of IL-1β were lower in the MS than controls. These findings were consistent with previous reports indicating a role for the inflammasome in the pathology of MS ^{6,}^{8,}¹¹.

### ASC and Caspase-1 are Good Serum Biomarkers of MS

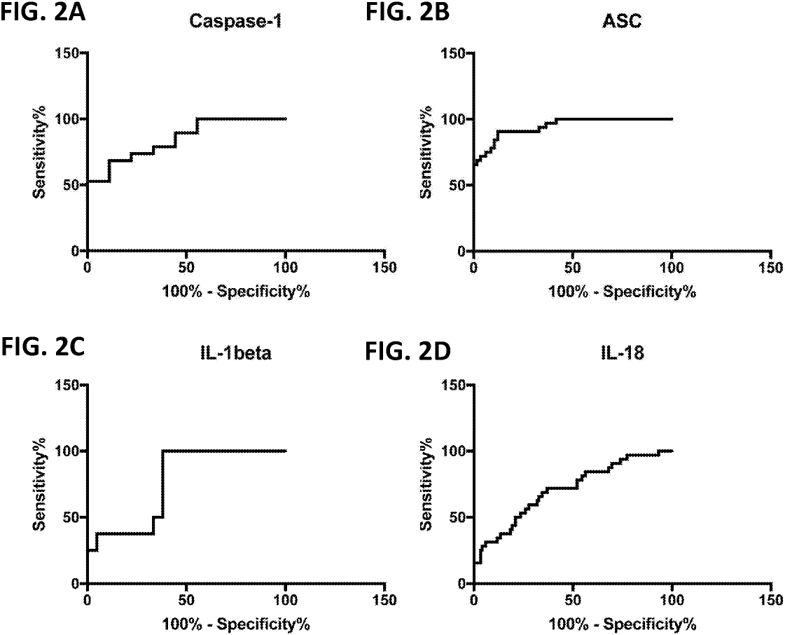

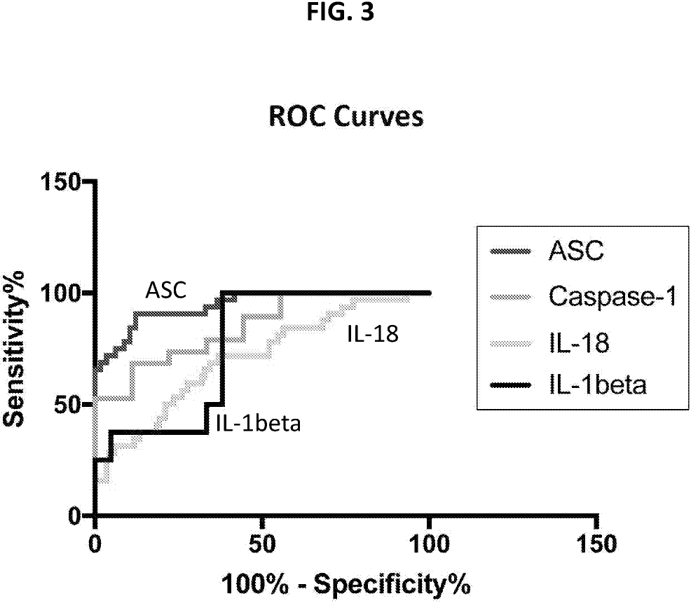

To then determine if these inflammasome signaling proteins have the potential to be reliable biomarkers for MS pathology, the area under the curve (AUC) for caspase-1 (FIG. 2A), ASC (FIG. 2B), IL-1beta (FIG. 2C) and IL-18 (FIG. 2D) were determined. Of the three proteins measured, ASC was shown to be the best biomarker (FIG. 3) with an AUC of 0.9448 and a CI between 0.9032 to 0.9864 (Table 1). In addition, caspase-1 with an AUC of 0.848 and a CI between 0.703 and 0.9929 is also promising biomarker of MS.

TABLE 1

ROC analysis results for inflammasome signaling proteins in serum

| BIOMARKER | AREA | STD. ERROR | 95% C.I. | P VALUE |
|---|---|---|---|---|
| Caspase-1 | 0.848 | 0.07394 | 0.703 to 0.9929 | 0.0034 |
| ASC | 0.9448 | 0.02122 | 0.9032 to 0.9864 | < 0.0001 |
| IL-1beta | 0.7619 | 0.0925 | 0.5806 to 0.9432 | 0.0318 |
| IL-18 | 0.7075 | 0.05216 | 0.6052 to 0.8097 | 0.0003 |

Furthermore, the cut-off point for ASC was 352.4 pg/ml with 84% sensitivity and 90% sensitivity (Table 2). For caspase-1, the cut-off point was 1.302 pg/ml with 89% sensitivity and 56% specificity (Table 2). Moreover, we found that in regards to ASC for a 100% sensitivity the cut-off point was 247.2 pg/ml with 58.26% specificity, and for 100% specificity, the cut-off point was 465.1 pg/ml and a 65.63% sensitivity. In the case of caspase-1, for 100% sensitivity, the cut-off point was 1.111 pg/ml with 44.44% specificity. For 100% specificity, the cut-off point was 2.718 pg/ml with 52.63% sensitivity. Thus, these findings indicate that caspase-1 and ASC can be biomarkers for MS.

TABLE 2

Cut-off point analyses for inflammasome signaling proteins in serum

| Biomarker | Cut-off point (pg/ml) | Sensitivity (%) | Specificity (%) |
|---|---|---|---|
| Caspase-1 | >1.302 | 89 | 56 |
| ASC | >352.4 | 84 | 90 |
| IL-1beta | <0.825 | 100 | 62 |
| IL-18 | >190.1 | 84 | 44 |

### Conclusions

In this study, a statistically significant higher level of IL-18 was detected in the serum of MS patients when compared to healthy subjects. In addition, the AUC for IL-18 in the cohort of patients was 0.7075 with a CI between 0.6052 to 0.8097 and a sensitivity of 84%, however, the specificity was only 44% when the cut-off point was 190.1 pg/ml. When the cut-off point was 104.2 pg/ml the sensitivity was 100% but the specificity was only 6.723%. Similarly, when the cut-off point was 427.2 pg/ml, the specificity was 100% but the sensitivity was only 15.63%.

Further, the levels of IL-1β were significantly lower in the MS group than the control group. The AUC was 0.7619 with a CI between 0.5806 to 0.9432. The sensitivity was 100% when the cut-off point was 0.825 with 62% specificity.

Higher protein levels of caspase-1 was also found in the serum of MS patients. Importantly, the AUC for caspase-1 was 0.848 with a CI between 0.703 to 0.9929. With a cut-off point of 1.302 pg/ml the sensitivity was 89% with 56% specificity. Moreover, with a 100% sensitivity the cut-off point was 1.111 pg/ml with 44.44% specificity; whereas with 100% specificity, the sensitivity was 52.63% with a cut-off point of 2.718 pg/ml.

Moreover, in this example, ASC was the most promising biomarker with an AUC of 0.9448 and a narrow CI between 0.9032 to 0.9864. A cut-off point of 352.4 pg/ml resulted in 84% sensitivity and 90% specificity. When the cut-off point was 247.2 pg/ml, the sensitivity was 100% and the specificity 58%.

Thus, based on these findings caspase-1 and ASC are promising biomarker with a high AUC value and a high sensitivity. Importantly, a combination of caspase-1 and ASC as biomarkers for MS with other diagnostic criteria may further increase the sensitivity of these biomarkers for MS beyond what is described in this example. Some clinically used biomarkers such as serum aquaporin 4 antibodies (AQP4-IgG), which is used to differentiate between patients with MS and patients with neuromyelitis optica, have a median sensitivity of 62.3% with a range between 12.5% to 100%, depending on the assay used for the measurements. ²⁹

Since the 1960s immunoglobulin (Ig) G oligoclonal bands (OCB) have been used as a classic biomarker in the diagnosis of MS. ³⁰ However, the specificity of IgG-OCB is only 61%, as a result, other diagnostic criteria is needed to clinically determine the diagnosis of MS, ³¹ yet CSF-restricted IgG-OCB is a good predictor for conversion from CIS to CDMS, independently of MRI ³². Similar results have been obtained when analyzing IgM-OCB. ³³ Interestingly, IgG against measles, rubella and varicella zoster (MRZ) are present in the CSF of MS patients, thus MRZ-specific IgG have the potential to be used as biomarkers of MS diagnosis. ³⁴

Importantly, in this study, caspase-1 and ASC have been identified as potential biomarkers of MS pathology with high AUC values; 0.9448 and 0.848, respectively with sensitivities above 80% and in the case of ASC a specificity of 90%.

### Incorporation by Reference

The following references are incorporated by reference in their entireties for all purposes.

1. Compston A. The pathogenesis and basis for treatment in multiple sclerosis. Clin Neurol Neurosurg. 2004;106:246-8.

2. de Rivero Vaccari JP, Lotocki G, Marcillo AE, Dietrich WD and Keane RW. A molecular platform in neurons regulates inflammation after spinal cord injury. J Neurosci. 2008;28:3404-14.

3. de Rivero Vaccari JP, Dietrich WD and Keane RW. Activation and regulation of cellular inflammasomes: gaps in our knowledge for central nervous system injury. J Cereb Blood Flow Metab. 2014;34:369-75.

4. Ming X, Li W, Maeda Y, Blumberg B, Raval S, Cook SD and Dowling PC. Caspase-1 expression in multiple sclerosis plaques and cultured glial cells. J Neurol Sci. 2002;197:9-18.

5. Cao Y, Goods BA, Raddassi K, Nepom GT, Kwok WW, Love JC and Hafler DA. Functional inflammatory profiles distinguish myelin-reactive T cells from patients with multiple sclerosis. Sci Transl Med. 2015;7:287ra74.

6. Furlan R, Martino G, Galbiati F, Poliani PL, Smiroldo S, Bergami A, Desina G, Comi G, Flavell R, Su MS and Adorini L. Caspase-1 regulates the inflammatory process leading to autoimmune demyelination. J Immunol. 1999;163:2403-9.

7. Inoue M, Williams KL, Gunn MD and Shinohara ML. NLRP3 inflammasome induces chemotactic immune cell migration to the CNS in experimental autoimmune encephalomyelitis. Proc Natl Acad Sci USA. 2012;109:10480-5.

8. Gris D, Ye Z, Iocca HA, Wen H, Craven RR, Gris P, Huang M, Schneider M, Miller SD and Ting JP. NLRP3 plays a critical role in the development of experimental autoimmune encephalomyelitis by mediating Th1 and Th17 responses. J Immunol. 2010;185:974-81.

9. Brand FJ, 3rd, Forouzandeh M, Kaur H, Travascio F and de Rivero Vaccari JP. Acidification changes affect the inflammasome in human nucleus pulposus cells. J Inflamm (Lond). 2016;13:29.

10. Xia J, Broadhurst DI, Wilson M and Wishart DS. Translational biomarker discovery in clinical metabolomics: an introductory tutorial. Metabolomics. 2013;9:280-299.

11. Dumas A, Amiable N, de Rivero Vaccari JP, Chae JJ, Keane RW, Lacroix S and Vallieres L. The inflammasome pyrin contributes to pertussis toxin-induced IL-1beta synthesis, neutrophil intravascular crawling and autoimmune encephalomyelitis. PLoS Pathog. 2014;10:e1004150.

12. Katsavos S and Anagnostouli M. Biomarkers in Multiple Sclerosis: An Up-to-Date Overview. Mult Scler Int. 2013;2013:340508.

13. Kuhle J, Disanto G, Dobson R, Adiutori R, Bianchi L, Topping J, Bestwick JP, Meier UC, Marta M, Dalla Costa G, Runia T, Evdoshenko E, Lazareva N, Thouvenot E, Iaffaldano P, Direnzo V, Khademi M, Piehl F, Comabella M, Sombekke M, Killestein J, Hegen H, Rauch S, D′Alfonso S, Alvarez-Cermeno JC, Kleinova P, Horakova D, Roesler R, Lauda F, Llufriu S, Avsar T, Uygunoglu U, Altintas A, Saip S, Menge T, Rajda C, Bergamaschi R, Moll N, Khalil M, Marignier R, Dujmovic I, Larsson H, Malmestrom C, Scarpini E, Fenoglio C, Wergeland S, Laroni A, Annibali V, Romano S, Martinez AD, Carra A, Salvetti M, Uccelli A, Torkildsen O, Myhr KM, Galimberti D, Rejdak K, Lycke J, Frederiksen JL, Drulovic J, Confavreux C, Brassat D, Enzinger C, Fuchs S, Bosca I, Pelletier J, Picard C, Colombo E, Franciotta D, Derfuss T, Lindberg R, Yaldizli O, Vecsei L, Kieseier BC, Hartung HP, Villoslada P, Siva A, Saiz A, Tumani H, Havrdova E, Villar LM, Leone M, Barizzone N, Deisenhammer F, Teunissen C, Montalban X, Tintore M, Olsson T, Trojano M, Lehmann S, Castelnovo G, Lapin S, Hintzen R, Kappos L, Furlan R, Martinelli V, Comi G, Ramagopalan SV and Giovannoni G. Conversion from clinically isolated syndrome to multiple sclerosis: A large multicentre study. Mult Scler. 2015;21:1013-24.

14. Lublin FD. New multiple sclerosis phenotypic classification. Eur Neurol. 2014;72 Suppl 1:1-5.

15. Milo R and Miller A. Revised diagnostic criteria of multiple sclerosis. Autoimmun Rev. 2014;13:518-24.

16. Inoue M, Chen PH, Siecinski S, Li QJ, Liu C, Steinman L, Gregory SG, Benner E and Shinohara ML. An interferon-beta-resistant and NLRP3 inflammasome-independent subtype of EAE with neuronal damage. Nat Neurosci. 2016;19:1599-1609.

17. Inoue M, Williams KL, Oliver T, Vandenabeele P, Rajan JV, Miao EA and Shinohara ML. Interferon-beta therapy against EAE is effective only when development of the disease depends on the NLRP3 inflammasome. Sci Signal. 2012;5:ra38.

18. Chen YC, Chen SD, Miao L, Liu ZG, Li W, Zhao ZX, Sun XJ, Jiang GX and Cheng Q. Serum levels of interleukin (IL)-18, IL-23 and IL-17 in Chinese patients with multiple sclerosis. J Neuroimmunol. 2012;243:56-60.

19. Losy J and Niezgoda A. IL-18 in patients with multiple sclerosis. Acta Neurol Scand. 2001;104:171-3.

20. Levesque SA, Pare A, Mailhot B, Bellver-Landete V, Kebir H, Lecuyer MA, Alvarez JI, Prat A, de Rivero Vaccari JP, Keane RW and Lacroix S. Myeloid cell transmigration across the CNS vasculature triggers IL-1beta-driven neuroinflammation during autoimmune encephalomyelitis in mice. J Exp Med. 2016;213:929-49.

21. Dujmovic I, Mangano K, Pekmezovic T, Quattrocchi C, Mesaros S, Stojsavljevic N, Nicoletti F and Drulovic J. The analysis of IL-1 beta and its naturally occurring inhibitors in multiple sclerosis: The elevation of IL-1 receptor antagonist and IL-1 receptor type II after steroid therapy. J Neuroimmunol. 2009;207:101-6.

22. Hauser SL, Doolittle TH, Lincoln R, Brown RH and Dinarello CA. Cytokine accumulations in CSF of multiple sclerosis patients: frequent detection of interleukin-1 and tumor necrosis factor but not interleukin-6. Neurology. 1990;40:1735-9.

23. Maimone D, Gregory S, Amason BG and Reder AT. Cytokine levels in the cerebrospinal fluid and serum of patients with multiple sclerosis. J Neuroimmunol. 1991;32:67-74.

24. Tsukada N, Miyagi K, Matsuda M, Yanagisawa N and Yone K. Tumor necrosis factor and interleukin-1 in the CSF and sera of patients with multiple sclerosis. J Neurol Sci. 1991;104:230-4.

25. Huang WX, Huang P and Hillert J. Increased expression of caspase-1 and interleukin-18 in peripheral blood mononuclear cells in patients with multiple sclerosis. Mult Scler. 2004;10:482-7.

26. de Rivero Vaccari JP, Dietrich WD and Keane RW. Therapeutics targeting the inflammasome after central nervous system injury. Transl Res. 2016;167:35-45.

27. de Rivero Vaccari JP, Lotocki G, Alonso OF, Bramlett HM, Dietrich WD and Keane RW. Therapeutic neutralization of the NLRP1 inflammasome reduces the innate immune response and improves histopathology after traumatic brain injury. J Cereb Blood Flow Metab. 2009;29:1251-61.

28. Shaw PJ, Lukens JR, Burns S, Chi H, McGargill MA and Kanneganti TD. Cutting edge: critical role for PYCARD/ASC in the development of experimental autoimmune encephalomyelitis. J Immunol. 2010;184:4610-4.

29. Jarius S and Wildemann B. Aquaporin-4 antibodies (NMO-IgG) as a serological marker of neuromyelitis optica: a critical review of the literature. Brain Pathol. 2013;23:661-83.

30. Stangel M, Fredrikson S, Meinl E, Petzold A, Stuve O and Tumani H. The utility of cerebrospinal fluid analysis in patients with multiple sclerosis. Nat Rev Neurol. 2013;9:267-76.

31. Teunissen CE, Malekzadeh A, Leurs C, Bridel C and Killestein J. Body fluid biomarkers for multiple sclerosis--the long road to clinical application. Nat Rev Neurol. 2015;11:585-96.

32. Tintore M, Rovira A, Rio J, Tur C, Pelayo R, Nos C, Tellez N, Perkal H, Comabella M, Sastre-Garriga J and Montalban X. Do oligoclonal bands add information to MRI in first attacks of multiple sclerosis? Neurology. 2008;70:1079-83.

33. Villar LM, Masjuan J, Gonzalez-Porque P, Plaza J, Sadaba MC, Roldan E, Bootello A and Alvarez-Cermeno JC. Intrathecal IgM synthesis predicts the onset of new relapses and a worse disease course in MS. Neurology. 2002;59:555-9.

34. Brettschneider J, Tumani H, Kiechle U, Muche R, Richards G, Lehmensiek V, Ludolph AC and Otto M. IgG antibodies against measles, rubella, and varicella zoster virus predict conversion to multiple sclerosis in clinically isolated syndrome. PLoS One. 2009;4:e7638.

### Example 2: Examination of Inflammasome Proteins as Biomarkers of Stroke Introduction

A biomarker is a characteristic that can be measured objectively and evaluated as an indicator of normal or pathologic biological processes⁹. Thus, in the context of stroke, biomarkers in blood or other body fluids can be used as indicators of stroke onset. However, to date, there is no biomarker available that is regularly used in the diagnosis and management of stroke. To this end, cytokines such as IL-10 or tumor necrosis factor as well as other inflammatory proteins such as C-reactive protein, high-mobility group box-1 or heat shock proteins have been considered as potential candidates for further biomarker analyses in stroke patients¹⁰⁻¹².

In this example, a Simple Plex Assay (Protein Simple) was used to analyze serum and serum-derived EV samples from stroke patients and control donors for inflammasome protein levels of caspase-1, apoptosis-associated speck-like protein containing a caspase-recruitment domain (ASC), Interleukin (IL)-1beta. Receiver operator characteristic (ROC) curves and associated confidence intervals were calculated following analysis of the serum and serum-derived EV samples from patients after stroke and from healthy unaffected donors to measure sensitivity and specificity of inflammasome proteins to establish the potential of inflammasome signaling proteins as biomarkers of stroke.

### Methods

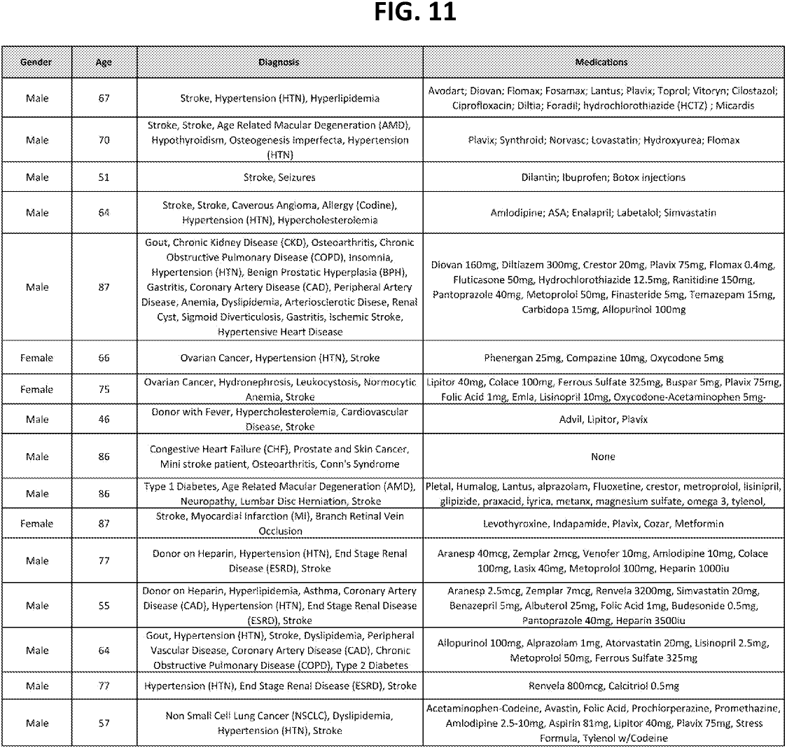

Participants: In this example, serum samples from 80 normal donors and 16 patients that were diagnosed with stroke were analyzed. Samples were purchased from BioreclamationIVT. The normal donor group consisted of samples obtained from 40 male and 40 female donors in the age range of 46 to 70 years old. The age range in the stroke group consisted of samples obtained from patients in the age range of 46 to 87 years old (FIG. 11).

### Isolation of EV

By Total Exosome Isolation from Serum kit (Invitrogen): Total Exosome Isolation from serum was used according to the manufacturer’s instructions (Invitrogen). Briefly, 100 ul of each sample was centrifuged at 2000 xg for 30 minutes. The supernatant was then incubated with 20 ul of Total Exosome Isolation reagent for 30 minutes at 4⁰ C followed by centrifugation at 10,000 xg for 10 minutes at room temperature. Supernatants were discarded and the pellet was resuspended in 50 ul of PBS.

By ExoQuick: EV were isolated from serum samples using ExoQuick (EQ, System Biosciences) as described in6. Briefly, 100 ul of each sample was centrifuged at 3,000 xg for 15 minutes. The supernatant was then incubated with 24.23 ul of ExoQuick Exosome Precipitation Solution (for serum) for 30 min at 4⁰ C followed by centrifugation at 1,500 xg for 30 minutes. Supernatants were discarded and residual EQ solution was centrifuged at 1,500 xg for 5 minutes. The pellet was then resuspended in 50 ul of PBS.

### Protein Assay

To determine the protein concentration of caspase-1, ASC, IL-1β and IL-18 in serum and serum-derived EV, a Simple Plex assay was run and analyzed with Simple Plex Explorer software. Results shown correspond to the mean of each sample run in triplicates. It should be noted that any system/instrument known in the art can be used to measure the levels of proteins (e.g., inflammasome proteins) in bodily fluids.

### Protein Quantification

To quantify the protein concentration in isolated EV, the Pierce Coomassie (Bradford) Protein Assay Kit (ThermoFisher Scienftific, Inc.) was used according to the manufacturer’s instructions. Serum-derived EV were lysed (1:1 dilution) in lysis buffer as described.⁶

### Nanoparticle Tracking Analysis (NTA)

EV were analyzed by NanoSight NS300 (Malvern Instruments Company, Nanosight, and Malvern, United Kingdom). Isolated exosomes were diluted in PBS (1:1000) for analysis, and three 90 second videos were then recorded. Data were analyzed using Nanosight NTA 2.3 Analytical Software (Malvern Instruments Company) with a detection threshold optimized for each sample and a screen gain set at 10 to track as many particles as possible while maintaining minimal background. At least three independent measurements were performed for each isolated sample.

### Immunoblotting

For detection of inflammasome signaling proteins in isolated EV, EV were resuspended in protein lysis buffer and resolved by immunoblotting as described in ¹⁵. Briefly, following lysis of the pellet proteins were resolved in 10-20% Criterion TGX Stain-Free precasted gels (Bio-Rad), using antibodies (1:1000 dilution) to NLRP3 (Novus Biologicals), caspase-1 (Novus Biologicals), ASC (Santa Cruz), IL-1beta (Cell Signaling), IL-18 (Abcam), CD81 (Thermo Scientific) and NCAM (Sigma). Quantification of band density was done using the UN-SCAN-IT gel 5.3 Software (Silk Scientific Corporation). Ten ul of sample was loaded. Chemilluminescence substrate (LumiGlo, Cell Signaling) in membranes was imaged using the ChemiDoc Touch Imaging System (BioRad).

### Gel Imaging

Total protein in the Criterion TGX Stain-Free precasted gels was imaged using the ChemiDoc Touch Imaging System (BioRad) by placing the gel in the tray of the ChemiDoc Touch following protein transfer. The image was then adjusted in the screen to show the entirety of the gel and running the Stain-Free Blot setting in the application window.

### Statistical Analyses

Statistical comparisons between the Invitrogen and ExoQuick isolation procedures were done using a two-tailed student t-test.

### Electron Microscopy Procedures

EV were loaded onto formvar-carbon coated grids. A 10 ul drop of the sample was then placed on clean parafilm and the grid was floated (face-down) for 30 min. Subsequent steps were also performed by floating the grid on a 10 ul bubble. The EV-loaded grid was then rinsed with 0.1 M Millonig’s phosphate buffer (Electron Microscopy Sciences) for 5 min. Excess fluid was drained. Then the grid was placed into 2% glutaraldehyde for 5 min. Subsequent washes were done to remove excess glutaraldehyde by rinsing with 0.1 M Millonig’s phosphate buffer for 5 min followed by distilled water for 2 min seven times on seven different bubbles. The grid was then transferred to a 0.4% Uranyl Acetate solution for 5 min. Grids were allowed to dry for imaging. Images were acquired with a Joel JEM-1400 transmission electron microscope, at a voltage of 80 kV, and a digital Gatan camera.

### Biomarker Analyses

Data were analyzed using Prism 7 software (GraphPad). Comparisons between groups for protein levels were carried by first identifying outliers followed by an unpaired t-test and then determining the area under the ROC curve, as well as the 95% confidence interval and the p-value (p-value of significance used was <0.05). Finally, sensitivity, specificity, positive predictive value (PPV), negative predictive value (NPV) and accuracy of each biomarker was obtained for a range of different cut-off points. Samples that yielded a protein value below the level of detection of the assay were not included in the analyses for that particular analyte.

### Results

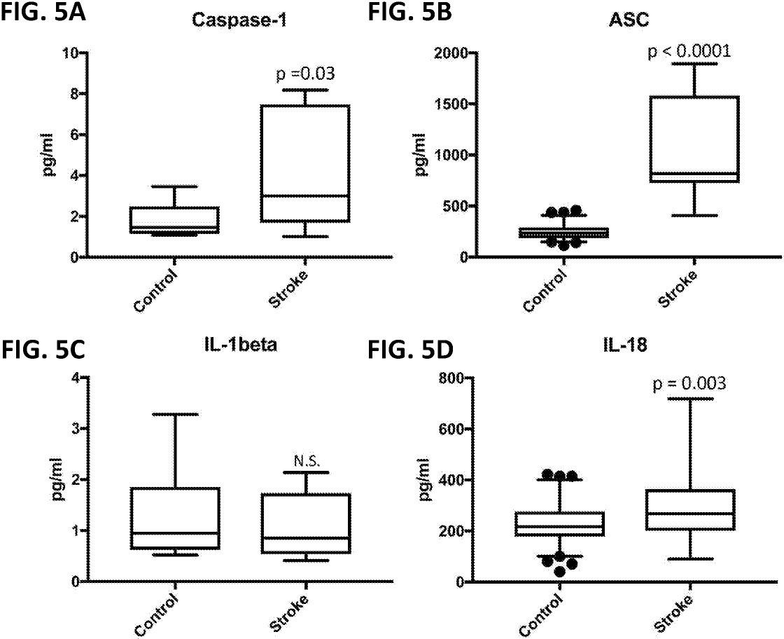

Caspase-1, ASC and IL-18 are elevated in the serum of stroke patients: To determine the protein levels of inflammasome proteins in serum from stroke patients and control donors, serum samples were analyzed with a Simple Plex system. Protein levels of caspase-1, ASC and IL-18 were higher in the serum of stroke patients when compared to the control samples, whereas levels of IL-1 were not significantly different (FIGS. 5A-5D). These findings confirm previous data showing that the inflammasome is involved in the inflammatory response after stroke^{4,}¹⁶.

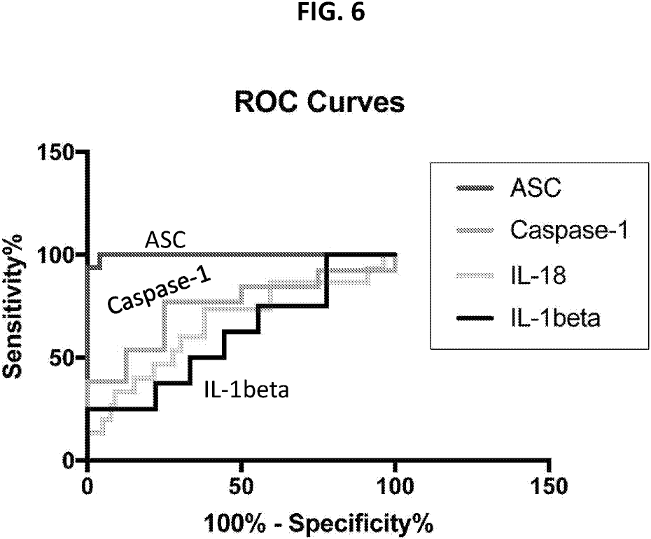

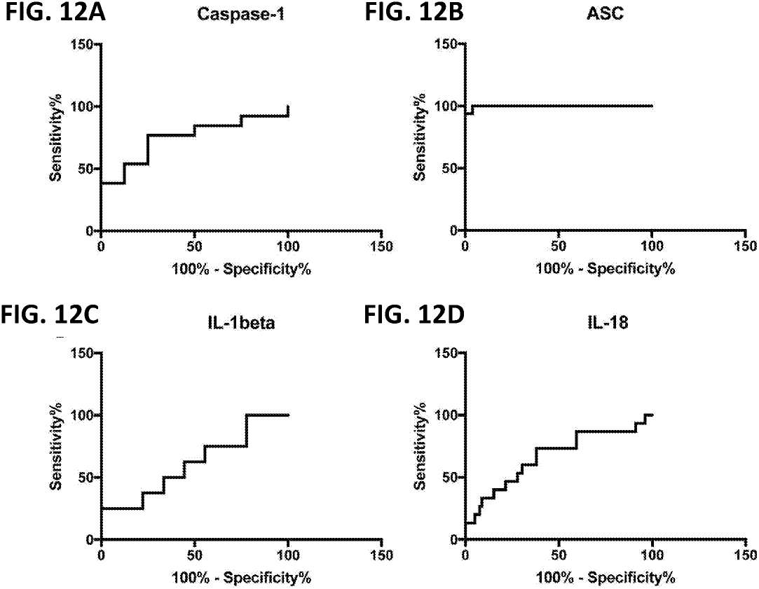

ASC as a serum biomarker of stroke: Higher levels of inflammasome proteins in serum from stroke patients may not be enough proof to show that inflammasome proteins are good biomarkers of stroke. Thus, an ROC analysis was performed (FIG. 6 and FIGS. 12A-12D) to determine the AUC. The AUC for ASC was 0.9975 with a confidence interval between 0.9914 to 1.004 (Table 3). The cut-off point for ASC was 404.8 pg/ml with a sensitivity of 100% and a specificity of 96% (Table 4). Thus, ASC appears to be a reliable biomarker of stroke.

TABLE 3

ROC analysis results for inflammasome signaling proteins in serum

| BIOMARKER | AREA | STD. ERROR | 95% C.I. | P VALUE |
|---|---|---|---|---|
| Caspase-1 | 0.75 | 0.1087 | 0.5369 to 0.9631 | 0.05 |
| ASC | 0.9975 | 0.003 | 0.9914 to 1.004 | < 0.0001 |
| IL-1beta | 0.6111 | 0.1407 | 0.3353 to 0.8869 | 0.44 |
| IL-18 | 0.6675 | 0.082 | 0.5059 to 0.8291 | 0.04 |

TABLE 4

Cut-off point analyses for inflammasome signaling proteins in serum

| Biomarker | Cut-off point (pg/ml) | Sensitivity (%) | Specificity (%) |
|---|---|---|---|
| Caspase-1 | > 1.412 | 85 | 50 |
| ASC | > 404.8 | 100 | 96 |
| IL-lbeta | < 0.984 | 63 | 56 |
| IL-18 | > 244.6 | 73 | 62 |

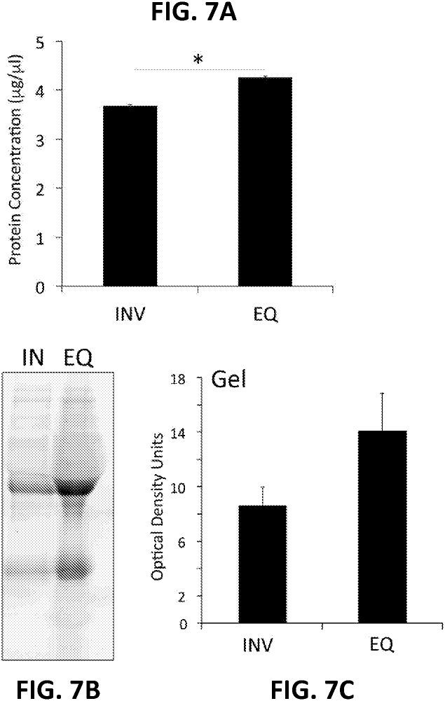

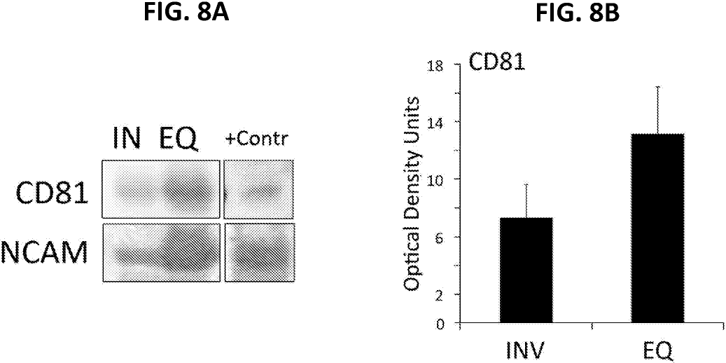

Amount of protein loaded in Isolated EV from stroke patients: To calculate the amount of protein present in the isolated exosomes from serum samples, a BCA assay was performed from isolates obtained by the Invitrogen method and the EQ method. The data indicated that the EQ method was able to isolate more protein than the Invitrogen method (FIGS. 7A-7C).

To visualize how much protein was loaded in a gel during immunoblot analysis, the Stain-Free Blot setting of the ChemiDoc Touch Imaging System was used. The representative image in FIG. 7B showed that when 10 ul was loaded of the serum-derived EV re-suspended in lysis buffer containing a protease inhibitor cocktail (Sigma), the lanes corresponding to the Invitrogen kit had less protein than the lane corresponding to the EQ kit; however, there was no statistical significant difference between the groups.

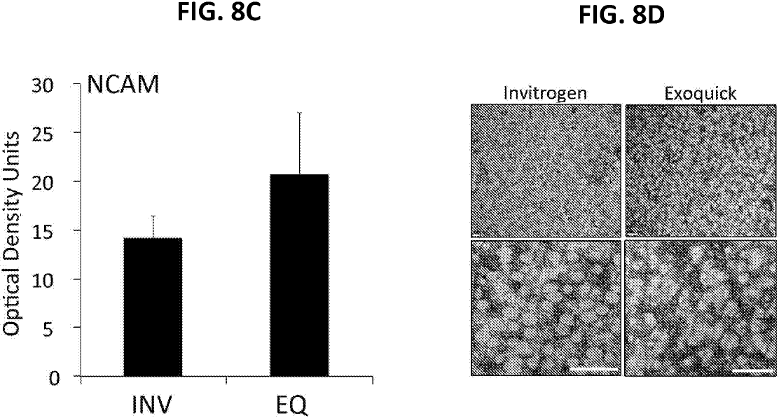

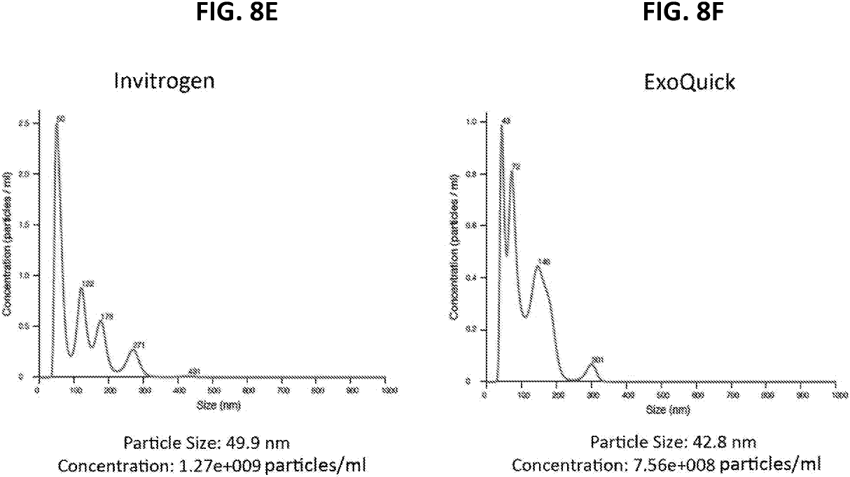

Invitrogen’s kit and EQ isolate CD81- and NCAM-positive EV from the serum of patients with stroke: To determine if inflammasome proteins present in EV are promising biomarkers of stroke, EV from the serum of stroke patients was isolated. Two different techniques of EV isolation was used to identify the most suitable method to isolate, inflammasome-containing EV. In addition, the tetraspanin protein CD81, a marker of EV {Andreu, 2014 #33} as well as and neural cell adhesion molecule (NCAM) a marker of neuronal-derived EV was used to demonstrate that the isolated EV are brain derived {Vella, 2016 #36}. Accordingly, both methods, the one from Invitrogen and EQ, were able to isolate CD81- and (NCAM)-positive EV (FIG. 8A). However, although the EQ seem to isolate higher levels of these proteins, there was no statistical significant difference between the two groups (FIG. 8B and FIG. 8C). EV-positive control isolate (System Biosciences) was run in parallel.

Electron microscopy was performed on the EV isolated by the two techniques and found that the Invitrogen kit gave more uniformed and round vesicles (FIG. 8D). In addition, NTA analyses revealed that the particle size was in the 40 to 50 nm range for both techniques, and the particle concentration of EV with the Invitrogen method was 1.27e+009 particles/ml and with EQ, 7.56+008 particles/ml (FIG. 8E and FIG. 8F). Taken together, based on the particle size and uniformity of vesicles, as determined by electron microscopy, it seems that the Invitrogen method is more suitable to isolate EV.

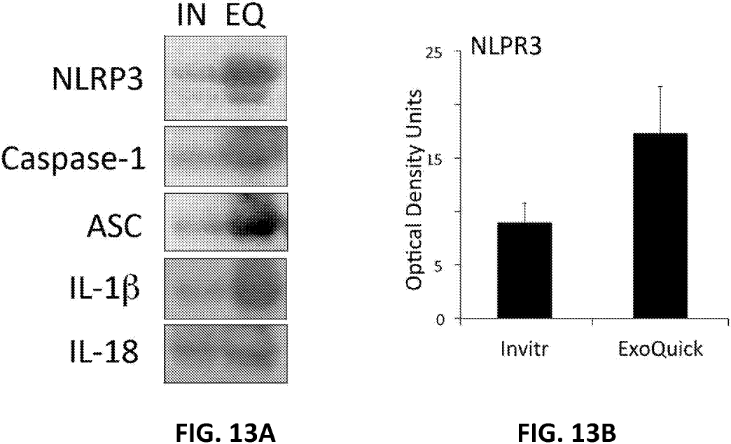

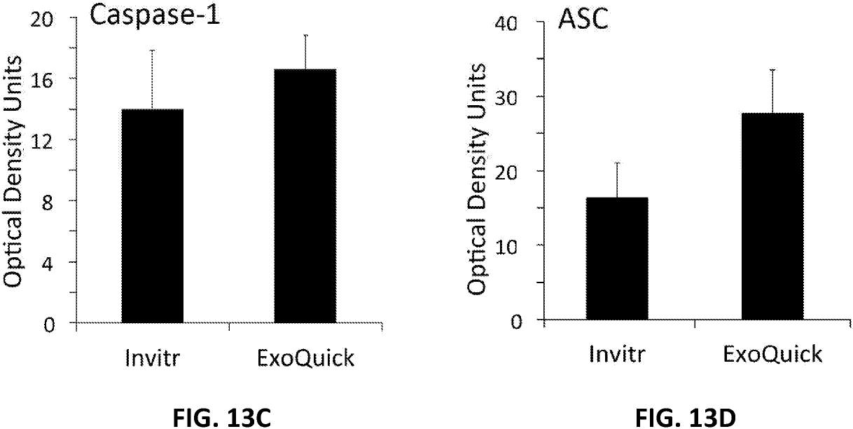

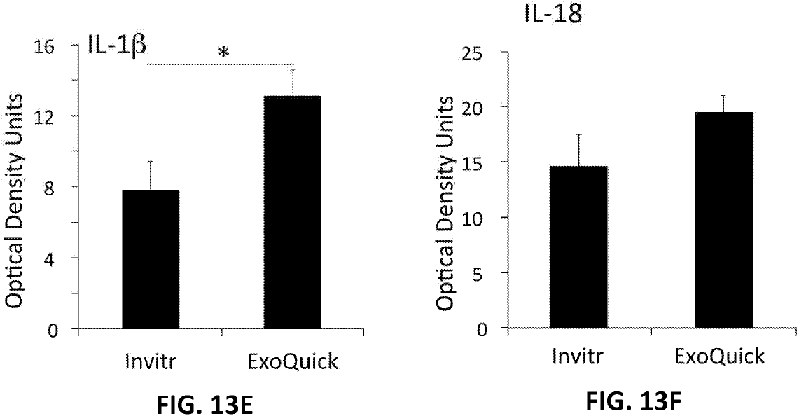

Invitrogen’s kit and EQ isolate inflammasome-positive EV from the serum of patients with stroke: It has been previously shown that inflammasome proteins are present in EV⁶. The levels of inflammasome protein expression was compared by the two different methods and found no statistical significant difference in NLPR3, caspase-1, ASC and IL-18 levels between the two different methods. However, the EQ method was able to isolate EV with higher levels of IL-1 beta than the Invitrogen method (see FIGS. 13A-13F).

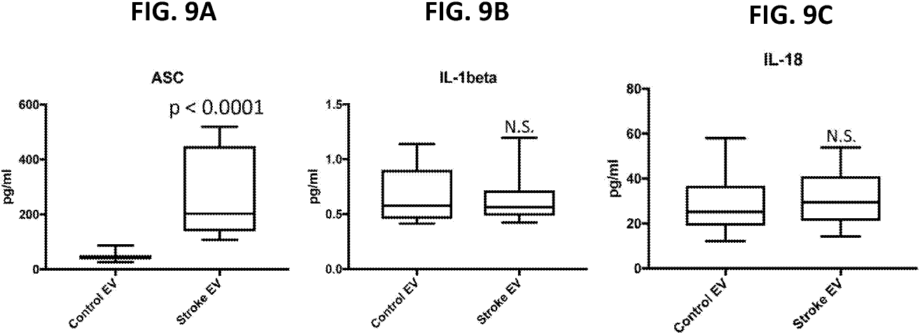

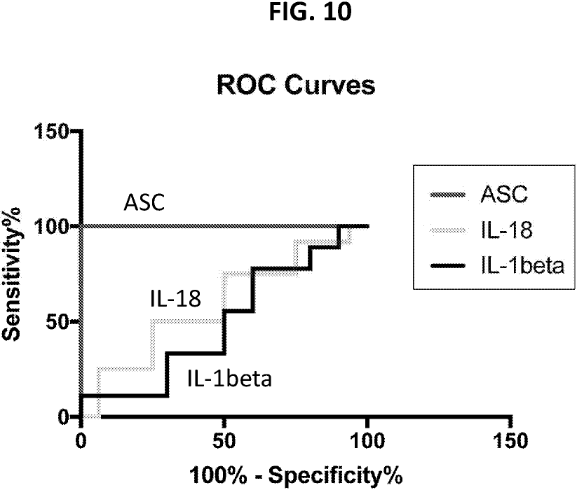

ASC is elevated in EV isolated from the serum of stroke patients: EV from the serum of 16 aged-matched donors and the 16 stroke samples (FIG. 11) was isolated and analyzed inflammasome protein levels in these isolated EV with the Simple Plex technology. The protein levels of ASC remained higher in serum-derived EV from stroke samples when compared to controls (FIGS. 9A-9C). However, the levels of IL-lbeta and IL-18 were not significantly different between the two groups, while the levels of caspase-1 in these isolated EV was below the limit of detection of these assay for this analyte.

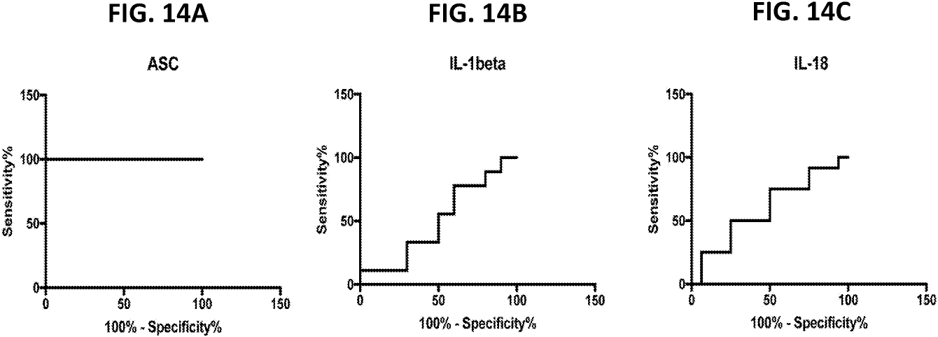

ASC in serum-derived EV is a good biomarker of stroke: To determine if inflammasome proteins in serum-derived EV can be viable biomarkers of stroke, an ROC analysis (see FIGS. 14A-14C) was conducted and found that ASC is a reliable biomarker of stroke (FIG. 10) with an AUC of 1 (Table 5) and a cut-off point of 97.57 pg/ml (Table 6).

TABLE 5

ROC analysis results for inflammasome signaling proteins in serum-derived EV

| BIOMARKER | AREA | STD. ERROR | 95% C.I. | P VALUE |
|---|---|---|---|---|
| ASC | 1 | 0 | 1 | < 0.0001 |
| IL-lbeta | 0.5 | 0.1375 | 0.2303 to 0.7697 | >0.9999 |
| IL-18 | 0.5938 | 0.1109 | 0.3763 to 0.8112 | 0.4034 |

TABLE 6

Cut-off analyses for inflammasome signaling proteins in serum-derived EV

| Biomarker | Cut-off point (pg/ml) | Sensitivity (%) | Specificity (%) |
|---|---|---|---|
| ASC | >97.57 | 100 | 100 |
| IL-lbeta | >0.5585 | 56 | 50 |
| IL-18 | >23.66 | 75 | 50 |

### Conclusion

In this example, it was shown that ASC is a reliable biomarker of stroke onset. The area under the curve (AUC) for ASC in serum was 0.9975 with a confidence interval between 0.9914 to 1.004. This AUC value was higher than the other inflammasome signaling proteins analyzed in this study: caspase-1 (0.75), IL-1beta (0.6111) and IL-18 (0.6675), indicating that ASC is a superior biomarker to the other inflammasome proteins that were looked at in this study. The cut-off point for ASC was 404.8 pg/ml with 100% sensitivity and a 96% specificity with the cohort of samples used. Importantly, the AUC was increased to 1 when analyzing serum-derived EV samples from a small subset of patients. Accordingly, the cut-off point for ASC in serum-derived EV was found to be 97.57 pg/ml.

In this study, the Invitrogen kit was able to provide better quality EV as visualized by electron microscopy and by NTA analysis of isolated vesicles, despite obtained higher levels of protein isolation with the EQ kit. Importantly, both methods were efficient at isolating EV containing inflammasome proteins

In conclusion, these studies highlight the potential of inflammasome proteins, particularly ASC as a biomarker of stroke in serum and serum-derived EV.

### Incorporation by Reference

The following references are incorporated by reference in their entireties for all purposes.

1. Xu X and Jiang Y. The Yin and Yang of innate immunity in stroke. Biomed Res Int. 2014;2014:807978.

2. Neumann S, Shields NJ, Balle T, Chebib M and Clarkson AN. Innate Immunity and Inflammation Post-Stroke: An alpha7-Nicotinic Agonist Perspective. Int J Mol Sci. 2015;16:29029-46.

3. Brand FJ, 3rd, de Rivero Vaccari JC, Mejias NH, Alonso OF and de Rivero Vaccari JP. RIG-I contributes to the innate immune response after cerebral ischemia. J Inflamm (Lond). 2015;12:52.

4. Abulafia DP, de Rivero Vaccari JP, Lozano JD, Lotocki G, Keane RW and Dietrich WD. Inhibition of the inflammasome complex reduces the inflammatory response after thromboembolic stroke in mice. J Cereb Blood Flow Metab. 2009;29:534-44.

5. de Rivero Vaccari JP, Dietrich WD and Keane RW. Therapeutics targeting the inflammasome after central nervous system injury. Transl Res. 2016;167:35-45.

6. de Rivero Vaccari JP, Brand F, 3rd, Adamczak S, Lee SW, Perez-Barcena J, Wang MY, Bullock MR, Dietrich WD and Keane RW. Exosome-mediated inflammasome signaling after central nervous system injury. J Neurochem. 2016;136 Suppl 1:39-48.

7. Zhang ZG and Chopp M. Exosomes in stroke pathogenesis and therapy. J Clin Invest. 2016; 126:1190-7.

8. Ji Q, Ji Y, Peng J, Zhou X, Chen X, Zhao H, Xu T, Chen L and Xu Y. Increased Brain-Specific MiR-9 and MiR-124 in the Serum Exosomes of Acute Ischemic Stroke Patients. PLoS One. 2016;11:e0163645.

9. Biomarkers Definitions Working G. Biomarkers and surrogate endpoints: preferred definitions and conceptual framework. Clin Pharmacol Ther. 2001;69:89-95.

10. Whiteley W, Chong WL, Sengupta A and Sandercock P. Blood markers for the prognosis of ischemic stroke: a systematic review. Stroke. 2009;40:e380-9.

11. Bustamante A, Simats A, Vilar-Bergua A, Garcia-Berrocoso T and Montaner J. Blood/Brain Biomarkers of Inflammation After Stroke and Their Association With Outcome: From C-Reactive Protein to Damage-Associated Molecular Patterns. Neurotherapeutics. 2016;13:671-684.

12. Katan M and Elkind MS. Inflammatory and neuroendocrine biomarkers of prognosis after ischemic stroke. Expert Rev Neurother. 2011;11:225-39.

13. Adamczak S, Dale G, de Rivero Vaccari JP, Bullock MR, Dietrich WD and Keane RW. Inflammasome proteins in cerebrospinal fluid of brain-injured patients as biomarkers of functional outcome: clinical article. J Neurosurg. 2012;117:1119-25.

14. Brand FJ, 3rd, Forouzandeh M, Kaur H, Travascio F and de Rivero Vaccari JP. Acidification changes affect the inflammasome in human nucleus pulposus cells. J Inflamm (Lond). 2016; 13:29.

15. de Rivero Vaccari JC, Brand FJ, 3rd, Berti AF, Alonso OF, Bullock MR and de Rivero Vaccari JP. Mincle signaling in the innate immune response after traumatic brain injury. Journal of neurotrauma. 2015;32:228-36.

16. de Rivero Vaccari JP, Patel HH, Brand FJ, 3rd, Perez-Pinzon MA, Bramlett HM and Raval AP. Estrogen receptor beta signaling alters cellular inflammasomes activity after global cerebral ischemia in reproductively senescence female rats. J Neurochem. 2016;136:492-6.

17. de Rivero Vaccari JP, Dietrich WD and Keane RW. Activation and regulation of cellular inflammasomes: gaps in our knowledge for central nervous system injury. J Cereb Blood Flow Metab. 2014;34:369-75.

18. de Rivero Vaccari JP, Dietrich WD and Keane RW. Therapeutics targeting the inflammasome after central nervous system injury. Translational research : the journal of laboratory and clinical medicine. 2015.

19. de Rivero Vaccari JP, Lotocki G, Alonso OF, Bramlett HM, Dietrich WD and Keane RW. Therapeutic neutralization of the NLRP1 inflammasome reduces the innate immune response and improves histopathology after traumatic brain injury. J Cereb Blood Flow Metab. 2009;29:1251-61.

20. de Rivero Vaccari JP, Lotocki G, Marcillo AE, Dietrich WD and Keane RW. A molecular platform in neurons regulates inflammation after spinal cord injury. J Neurosci. 2008;28:3404-14.

21. Fann DY, Lee SY, Manzanero S, Tang SC, Gelderblom M, Chunduri P, Bernreuther C, Glatzel M, Cheng YL, Thundyil J, Widiapradja A, Lok KZ, Foo SL, Wang YC, Li YI, Drummond GR, Basta M, Magnus T, Jo DG, Mattson MP, Sobey CG and Arumugam TV. Intravenous immunoglobulin suppresses NLRP1 and NLRP3 inflammasome-mediated neuronal death in ischemic stroke. Cell Death Dis. 2013;4:e790.

22. Minkiewicz J, de Rivero Vaccari JP and Keane RW. Human astrocytes express a novel NLRP2 inflammasome. Glia. 2013;61:1113-21.

23. Sun X, Song X, Zhang L, Sun J, Wei X, Meng L and An J. NLRP2 is highly expressed in a mouse model of ischemic stroke. Biochem Biophys Res Commun. 2016;479:656-662.

24. Ma Q, Chen S, Hu Q, Feng H, Zhang JH and Tang J. NLRP3 inflammasome contributes to inflammation after intracerebral hemorrhage. Ann Neurol. 2014;75:209-19.

25. Fann DY, Lim YA, Cheng YL, Lok KZ, Chunduri P, Baik SH, Drummond GR, Dheen ST, Sobey CG, Jo DG, Chen CL and Arumugam TV. Evidence that NF-kappaB and MAPK Signaling Promotes NLRP Inflammasome Activation in Neurons Following Ischemic Stroke. Mol Neurobiol. 2017.

26. Zhang N, Zhang X, Liu X, Wang H, Xue J, Yu J, Kang N and Wang X. Chrysophanol inhibits NALP3 inflammasome activation and ameliorates cerebral ischemia/reperfusion in mice. Mediators Inflamm. 2014;2014:370530.

27. Mendis S, Davis S and Norrving B. Organizational update: the world health organization global status report on noncommunicable diseases 2014; one more landmark step in the combat against stroke and vascular disease. Stroke. 2015;46:e121-2.

28. Esenwa CC and Elkind MS. Inflammatory risk factors, biomarkers and associated therapy in ischaemic stroke. Nat Rev Neurol. 2016;12:594-604.

29. Ridker PM and Haughie P. Prospective studies of C-reactive protein as a risk factor for cardiovascular disease. J Investig Med. 1998;46:391-5.

30. Rosenson RS and Stafforini DM. Modulation of oxidative stress, inflammation, and atherosclerosis by lipoprotein-associated phospholipase A2. J Lipid Res. 2012;53:1767-82.

31. Oei HH, van der Meer IM, Hofman A, Koudstaal PJ, Stijnen T, Breteler MM and Witteman JC. Lipoprotein-associated phospholipase A2 activity is associated with risk of coronary heart disease and ischemic stroke: the Rotterdam Study. Circulation. 2005; 111:570-5.

33. Barber M, Langhome P, Rumley A, Lowe GD and Stott DJ. Hemostatic function and progressing ischemic stroke: D-dimer predicts early clinical progression. Stroke. 2004;35:1421-5.

34. Turaj W, Slowik A, Dziedzic T, Pulyk R, Adamski M, Strojny J and Szczudlik A. Increased plasma fibrinogen predicts one-year mortality in patients with acute ischemic stroke. J Neurol Sci. 2006;246:13-9.

35. Mathivanan S, Ji H and Simpson RJ. Exosomes: extracellular organelles important in intercellular communication. J Proteomics. 2010;73:1907-20.

36. Le Pecq JB. Dexosomes as a therapeutic cancer vaccine: from bench to bedside. Blood Cells Mol Dis. 2005;35:129-35.

37. Kourembanas S. Exosomes: vehicles of intercellular signaling, biomarkers, and vectors of cell therapy. Annu Rev Physiol. 2015;77:13-27.

38. Thery C, Zitvogel L and Amigorena S. Exosomes: composition, biogenesis and function. Nat Rev Immunol. 2002;2:569-79.

39. Campos JH, Soares RP, Ribeiro K, Andrade AC, Batista WL and Torrecilhas AC. Extracellular Vesicles: Role in Inflammatory Responses and Potential Uses in Vaccination in Cancer and Infectious Diseases. J Immunol Res. 2015;2015:832057.

40. Hurley JH, Boura E, Carlson LA and Rozycki B. Membrane budding. Cell. 2010;143:875-87.

41. Thery C, Ostrowski M and Segura E. Membrane vesicles as conveyors of immune responses. Nat Rev Immunol. 2009;9:581-93.

42. Vella LJ, Sharples RA, Nisbet RM, Cappai R and Hill AF. The role of exosomes in the processing of proteins associated with neurodegenerative diseases. Eur Biophys J. 2008;37:323-32.

43. Izquierdo-Useros N, Naranjo-Gomez M, Erkizia I, Puertas MC, Borras FE, Blanco J and Martinez-Picado J. HIV and mature dendritic cells: Trojan exosomes riding the Trojan horse? PLoS Pathog. 2010;6:e1000740.

44. Luga V, Zhang L, Viloria-Petit AM, Ogunjimi AA, Inanlou MR, Chiu E, Buchanan M, Hosein AN, Basik M and Wrana JL. Exosomes mediate stromal mobilization of autocrine Wnt-PCP signaling in breast cancer cell migration. Cell. 2012;151:1542-56.

45. Robbins PD and Morelli AE. Regulation of immune responses by extracellular vesicles. Nat Rev Immunol. 2014; 14:195-208.

46. Rekker K, Saare M, Roost AM, Kubo AL, Zarovni N, Chiesi A, Salumets A and Peters M. Comparison of serum exosome isolation methods for microRNA profiling. Clin Biochem. 2014;47:135-8.

47. Taylor DD, Zacharias W and Gercel-Taylor C. Exosome isolation for proteomic analyses and RNA profiling. Methods Mol Biol. 2011;728:235-46.

48. Caradec J, Kharmate G, Hosseini-Beheshti E, Adomat H, Gleave M and Guns E. Reproducibility and efficiency of serum-derived exosome extraction methods. Clin Biochem. 2014;47:1286-92.

TABLE 7

Cut-off values for ASC levels in serum for Multiple Sclerosis (MS)

| Cutoff (pg/ml) | Sensitivity% | 95% CI | Specificity% | 95% CI | Likelihood ratio |
|---|---|---|---|---|---|
| > 105.8 | 100 | 89.11% to 100% | 0.8696 | 0.02201% to 4.75% | 1.009 |
| > 107.9 | 100 | 89.11% to 100% | 1.739 | 0.2113% to 6.141% | 1.018 |
| > 112.1 | 100 | 89.11% to 100% | 2.609 | 0.5412% to 7.435% | 1.027 |
| > 123.3 | 100 | 89.11% to 100% | 3.478 | 0.9557% to 8.667% | 1.036 |
| > 132.4 | 100 | 89.11% to 100% | 4.348 | 1.427% to 9.855% | 1.045 |
| > 133 | 100 | 89.11% to 100% | 5.217 | 1.939% to 11.01% | 1.055 |
| > 134.2 | 100 | 89.11% to 100% | 6.087 | 2.482% to 12.14% | 1.065 |
| > 135.2 | 100 | 89.11% to 100% | 6.957 | 3.051% to 13.25% | 1.075 |
| > 135.5 | 100 | 89.11% to 100% | 7.826 | 3.641% to 14.34% | 1.085 |
| > 135.8 | 100 | 89.11% to 100% | 8.696 | 4.249% to 15.41% | 1.095 |
| > 136.1 | 100 | 89.11% to 100% | 9.565 | 4.872% to 16.47% | 1.106 |
| > 139.2 | 100 | 89.11% to 100% | 10.43 | 5.509% to 17.52% | 1.117 |
| > 142.6 | 100 | 89.11% to 100% | 11.3 | 6.158% to 18.55% | 1.127 |
| > 143.3 | 100 | 89.11% to 100% | 12.17 | 6.818% to 19.58% | 1.139 |
| > 144.6 | 100 | 89.11% to 100% | 13.04 | 7.488% to 20.6% | 1.15 |
| > 146.2 | 100 | 89.11% to 100% | 13.91 | 8.167% to 21.61% | 1.162 |
| > 147.5 | 100 | 89.11% to 100% | 14.78 | 8.854% to 22.61% | 1.173 |
| > 148.9 | 100 | 89.11% to 100% | 15.65 | 9.548% to 23.6% | 1.186 |
| > 150.4 | 100 | 89.11% to 100% | 16.52 | 10.25% to 24.59% | 1.198 |
| > 151.4 | 100 | 89.11% to 100% | 17.39 | 10.96% to 25.57% | 1.211 |
| > 151.8 | 100 | 89.11% to 100% | 18.26 | 11.67% to 26.55% | 1.223 |
| > 154.3 | 100 | 89.11% to 100% | 19.13 | 12.39% to 27.52% | 1.237 |
| > 158.2 | 100 | 89.11% to 100% | 20 | 13.12% to 28.48% | 1.25 |
| > 160.8 | 100 | 89.11% to 100% | 20.87 | 13.85% to 29.44% | 1.264 |
| > 164 | 100 | 89.11% to 100% | 21.74 | 14.59% to 30.4% | 1.278 |
| > 168 | 100 | 89.11% to 100% | 22.61 | 15.33% to 31.35% | 1.292 |
| > 170.2 | 100 | 89.11% to 100% | 23.48 | 16.08% to 32.29% | 1.307 |
| > 171.2 | 100 | 89.11% to 100% | 24.35 | 16.83% to 33.23% | 1.322 |
| > 172.2 | 100 | 89.11% to 100% | 25.22 | 17.58% to 34.17% | 1.337 |
| > 173.4 | 100 | 89.11% to 100% | 26.09 | 18.34% to 35.1% | 1.353 |
| > 175.6 | 100 | 89.11% to 100% | 26.96 | 19.11% to 36.03% | 1.369 |
| > 178.5 | 100 | 89.11% to 100% | 27.83 | 19.87% to 36.95% | 1.386 |
| > 180.9 | 100 | 89.11% to 100% | 28.7 | 20.65% to 37.88% | 1.402 |
| > 182.1 | 100 | 89.11% to 100% | 29.57 | 21.42% to 38.79% | 1.42 |
| > 183.3 | 100 | 89.11% to 100% | 30.43 | 22.2% to 39.71% | 1.438 |
| > 184.4 | 100 | 89.11% to 100% | 31.3 | 22.98% to 40.62% | 1.456 |
| > 184.9 | 100 | 89.11% to 100% | 32.17 | 23.77% to 41.53% | 1.474 |
| > 185.7 | 100 | 89.11% to 100% | 33.04 | 24.56% to 42.43% | 1.494 |
| > 186.5 | 100 | 89.11% to 100% | 33.91 | 25.35% to 43.33% | 1.513 |
| > 188.9 | 100 | 89.11% to 100% | 34.78 | 26.14% to 44.23% | 1.533 |
| > 191.1 | 100 | 89.11% to 100% | 35.65 | 26.94% to 45.12% | 1.554 |
| > 191.9 | 100 | 89.11% to 100% | 36.52 | 27.74% to 46.01% | 1.575 |
| > 193.1 | 100 | 89.11% to 100% | 37.39 | 28.55% to 46.9% | 1.597 |
| > 195.2 | 100 | 89.11% to 100% | 38.26 | 29.35% to 47.79% | 1.62 |
| > 196.6 | 100 | 89.11% to 100% | 39.13 | 30.16% to 48.67% | 1.643 |
| > 197.2 | 100 | 89.11% to 100% | 40 | 30.98% to 49.55% | 1.667 |
| > 198.7 | 100 | 89.11% to 100% | 40.87 | 31.79% to 50.43% | 1.691 |
| > 202.1 | 100 | 89.11% to 100% | 41.74 | 32.61% to 51.3% | 1.716 |
| > 207.2 | 100 | 89.11% to 100% | 42.61 | 33.44% to 52.17% | 1.742 |
| > 210 | 100 | 89.11% to 100% | 43.48 | 34.26% to 53.04% | 1.769 |
| > 211.1 | 100 | 89.11% to 100% | 44.35 | 35.09% to 53.91% | 1.797 |
| > 214.3 | 100 | 89.11% to 100% | 45.22 | 35.92% to 54.77% | 1.825 |
| > 216.8 | 100 | 89.11% to 100% | 46.09 | 36.75% to 55.63% | 1.855 |
| > 218.1 | 100 | 89.11% to 100% | 46.96 | 37.59% to 56.49% | 1.885 |
| > 220.4 | 100 | 89.11% to 100% | 47.83 | 38.43% to 57.34% | 1.917 |
| > 224.1 | 100 | 89.11% to 100% | 48.7 | 39.27% to 58.19% | 1.949 |
| > 227.1 | 100 | 89.11% to 100% | 49.57 | 40.11% to 59.04% | 1.983 |
| > 228.8 | 100 | 89.11% to 100% | 50.43 | 40.96% to 59.89% | 2.018 |
| > 230.8 | 100 | 89.11% to 100% | 51.3 | 41.81% to 60.73% | 2.054 |
| > 231.7 | 100 | 89.11% to 100% | 52.17 | 42.66% to 61.57% | 2.091 |
| > 232.6 | 100 | 89.11% to 100% | 53.04 | 43.51% to 62.41% | 2.13 |
| > 233.5 | 100 | 89.11% to 100% | 53.91 | 44.37% to 63.25% | 2.17 |
| > 238.2 | 100 | 89.11% to 100% | 54.78 | 45.23% to 64.08% | 2.212 |
| > 243.1 | 100 | 89.11% to 100% | 55.65 | 46.09% to 64.91% | 2.255 |
| > 244 | 100 | 89.11% to 100% | 56.52 | 46.96% to 65.74% | 2.3 |
| > 244.7 | 100 | 89.11% to 100% | 57.39 | 47.83% to 66.56% | 2.347 |
| > 247.2 | 100 | 89.11% to 100% | 58.26 | 48.7% to 67.39% | 2.396 |
| > 249.6 | 96.88 | 83.78% to 99.92% | 58.26 | 48.7% to 67.39% | 2.321 |
| > 250.2 | 96.88 | 83.78% to 99.92% | 59.13 | 49.57% to 68.21% | 2.37 |
| > 250.5 | 96.88 | 83.78% to 99.92% | 60 | 50.45% to 69.02% | 2.422 |
| > 250.7 | 96.88 | 83.78% to 99.92% | 60.87 | 51.33% to 69.84% | 2.476 |
| > 251.6 | 96.88 | 83.78% to 99.92% | 61.74 | 52.21% to 70.65% | 2.532 |
| > 252.4 | 96.88 | 83.78% to 99.92% | 62.61 | 53.1% to 71.45% | 2.591 |
| > 253.2 | 96.88 | 83.78% to 99.92% | 63.48 | 53.99% to 72.26% | 2.653 |
| > 254.9 | 93.75 | 79.19% to 99.23% | 63.48 | 53.99% to 72.26% | 2.567 |
| > 257.2 | 93.75 | 79.19% to 99.23% | 64.35 | 54.88% to 73.06% | 2.63 |
| > 259 | 93.75 | 79.19% to 99.23% | 65.22 | 55.77% to 73.86% | 2.695 |
| > 260.8 | 93.75 | 79.19% to 99.23% | 66.09 | 56.67% to 74.65% | 2.764 |
| > 263 | 93.75 | 79.19% to 99.23% | 66.96 | 57.57% to 75.44% | 2.837 |
| > 264.2 | 90.63 | 74.98% to 98.02% | 66.96 | 57.57% to 75.44% | 2.743 |
| > 267.1 | 90.63 | 74.98% to 98.02% | 67.83 | 58.47% to 76.23% | 2.817 |
| > 270.9 | 90.63 | 74.98% to 98.02% | 68.7 | 59.38% to 77.02% | 2.895 |
| > 272.3 | 90.63 | 74.98% to 98.02% | 69.57 | 60.29% to 77.8% | 2.978 |
| > 272.7 | 90.63 | 74.98% to 98.02% | 70.43 | 61.21% to 78.58% | 3.065 |
| > 273.3 | 90.63 | 74.98% to 98.02% | 71.3 | 62.12% to 79.35% | 3.158 |
| > 277.9 | 90.63 | 74.98% to 98.02% | 72.17 | 63.05% to 80.13% | 3.257 |
| > 282.9 | 90.63 | 74.98% to 98.02% | 73.04 | 63.97% to 80.89% | 3.362 |
| > 283.9 | 90.63 | 74.98% to 98.02% | 73.91 | 64.9% to 81.66% | 3.474 |
| > 286.3 | 90.63 | 74.98% to 98.02% | 74.78 | 65.83% to 82.42% | 3.594 |
| > 289.3 | 90.63 | 74.98% to 98.02% | 75.65 | 66.77% to 83.17% | 3.722 |
| > 290.4 | 90.63 | 74.98% to 98.02% | 76.52 | 67.71% to 83.92% | 3.86 |
| > 294.2 | 90.63 | 74.98% to 98.02% | 77.39 | 68.65% to 84.67% | 4.008 |
| > 298 | 90.63 | 74.98% to 98.02% | 78.26 | 69.6% to 85.41% | 4.169 |
| > 300.4 | 90.63 | 74.98% to 98.02% | 79.13 | 70.56% to 86.15% | 4.342 |
| > 302.7 | 90.63 | 74.98% to 98.02% | 80 | 71.52% to 86.88% | 4.531 |
| > 304 | 90.63 | 74.98% to 98.02% | 80.87 | 72.48% to 87.61% | 4.737 |
| > 310.4 | 90.63 | 74.98% to 98.02% | 81.74 | 73.45% to 88.33% | 4.963 |
| > 318.3 | 90.63 | 74.98% to 98.02% | 82.61 | 74.43% to 89.04% | 5.211 |
| > 321.9 | 90.63 | 74.98% to 98.02% | 83.48 | 75.41% to 89.75% | 5.485 |
| > 324.4 | 90.63 | 74.98% to 98.02% | 84.35 | 76.4% to 90.45% | 5.79 |
| > 326.2 | 90.63 | 74.98% to 98.02% | 85.22 | 77.39% to 91.15% | 6.131 |
| > 328.7 | 90.63 | 74.98% to 98.02% | 86.09 | 78.39% to 91.83% | 6.514 |
| > 331 | 90.63 | 74.98% to 98.02% | 86.96 | 79.4% to 92.51% | 6.948 |
| > 335.3 | 90.63 | 74.98% to 98.02% | 87.83 | 80.42% to 93.18% | 7.444 |
| > 343.6 | 87.5 | 71.01% to 96.49% | 87.83 | 80.42% to 93.18% | 7.188 |
| > 349 | 84.38 | 67.21% to 94.72% | 87.83 | 80.42% to 93.18% | 6.931 |
| > 351.1 | 84.38 | 67.21% to 94.72% | 88.7 | 81.45% to 93.84% | 7.464 |
| > 352.4 | 84.38 | 67.21% to 94.72% | 89.57 | 82.48% to 94.49% | 8.086 |
| > 353.5 | 81.25 | 63.56% to 92.79% | 89.57 | 82.48% to 94.49% | 7.786 |
| > 354.2 | 78.13 | 60.03% to 90.72% | 89.57 | 82.48% to 94.49% | 7.487 |
| > 356.7 | 78.13 | 60.03% to 90.72% | 90.43 | 83.53% to 95.13% | 8.168 |
| > 364.1 | 78.13 | 60.03% to 90.72% | 91.3 | 84.59% to 95.75% | 8.984 |
| > 375.2 | 75 | 56.6% to 88.54% | 91.3 | 84.59% to 95.75% | 8.625 |
| > 381.9 | 75 | 56.6% to 88.54% | 92.17 | 85.66% to 96.36% | 9.583 |
| > 383.7 | 75 | 56.6% to 88.54% | 93.04 | 86.75% to 96.95% | 10.78 |
| > 386.6 | 75 | 56.6% to 88.54% | 93.91 | 87.86% to 97.52% | 12.32 |
| > 391.8 | 71.88 | 53.25% to 86.25% | 93.91 | 87.86% to 97.52% | 11.81 |
| > 396.9 | 71.88 | 53.25% to 86.25% | 94.78 | 88.99% to 98.06% | 13.78 |
| > 400.4 | 71.88 | 53.25% to 86.25% | 95.65 | 90.15% to 98.57% | 16.53 |
| > 406.6 | 71.88 | 53.25% to 86.25% | 96.52 | 91.33% to 99.04% | 20.66 |
| > 423.8 | 68.75 | 49.99% to 83.88% | 96.52 | 91.33% to 99.04% | 19.77 |
| > 437.2 | 68.75 | 49.99% to 83.88% | 97.39 | 92.57% to 99.46% | 26.35 |
| > 437.7 | 68.75 | 49.99% to 83.88% | 98.26 | 93.86% to 99.79% | 39.53 |
| > 441 | 65.63 | 46.81% to 81.43% | 98.26 | 93.86% to 99.79% | 37.73 |
| > 451.3 | 65.63 | 46.81% to 81.43% | 99.13 | 95.25% to 99.98% | 75.47 |
| > 465.1 | 65.63 | 46.81% to 81.43% | 100 | 96.84% to 100% | |
| > 475.7 | 62.5 | 43.69% to 78.9% | 100 | 96.84% to 100% | |
| > 480.7 | 59.38 | 40.64% to 76.3% | 100 | 96.84% to 100% | |
| > 501.8 | 56.25 | 37.66% to 73.64% | 100 | 96.84% to 100% | |
| > 522.9 | 53.13 | 34.74% to 70.91% | 100 | 96.84% to 100% | |
| > 537.5 | 50 | 31.89% to 68.11% | 100 | 96.84% to 100% | |
| > 560.5 | 46.88 | 29.09% to 65.26% | 100 | 96.84% to 100% | |
| > 575.6 | 43.75 | 26.36% to 62.34% | 100 | 96.84% to 100% | |
| > 621.7 | 40.63 | 23.7% to 59.36% | 100 | 96.84% to 100% | |
| > 698.9 | 37.5 | 21.1% to 56.31% | 100 | 96.84% to 100% | |
| > 740.4 | 34.38 | 18.57% to 53.19% | 100 | 96.84% to 100% | |
| > 758.3 | 31.25 | 16.12% to 50.01% | 100 | 96.84% to 100% | |
| > 814.6 | 28.13 | 13.75% to 46.75% | 100 | 96.84% to 100% | |
| > 866.6 | 25 | 11.46% to 43.4% | 100 | 96.84% to 100% | |
| > 888.7 | 21.88 | 9.277% to 39.97% | 100 | 96.84% to 100% | |
| > 910.2 | 18.75 | 7.208% to 36.44% | 100 | 96.84% to 100% | |
| > 927.1 | 15.63 | 5.275% to 32.79% | 100 | 96.84% to 100% | |
| > 947 | 12.5 | 3.513% to 28.99% | 100 | 96.84% to 100% | |
| > 961.3 | 9.375 | 1.977% to 25.02% | 100 | 96.84% to 100% | |
| > 1252 | 6.25 | 0.7661% to 20.81% | 100 | 96.84% to 100% | |
| > 1668 | 3.125 | 0.07909% to 16.22% | 100 | 96.84% to 100% | |

TABLE 8

Cut-off values for ASC levels in serum for Stroke

| Cutoff (pg/ml) | Sensitivity% | 95% CI | Specificity% | 95% CI | Likelihood ratio |
|---|---|---|---|---|---|
| > 128.7 | 100 | 79.41% to 100% | 1.333 | 0.03375% to 7.206% | 1.014 |
| > 145.8 | 100 | 79.41% to 100% | 2.667 | 0.3246% to 9.303% | 1.027 |
| > 148.9 | 100 | 79.41% to 100% | 4 | 0.8326% to 11.25% | 1.042 |
| > 150.4 | 100 | 79.41% to 100% | 5.333 | 1.472% to 13.1% | 1.056 |
| > 153.9 | 100 | 79.41% to 100% | 6.667 | 2.2% to 14.88% | 1.071 |
| > 158.2 | 100 | 79.41% to 100% | 8 | 2.993% to 16.6% | 1.087 |
| > 164.8 | 100 | 79.41% to 100% | 9.333 | 3.835% to 18.29% | 1.103 |
| > 170.2 | 100 | 79.41% to 100% | 10.67 | 4.719% to 19.94% | 1.119 |
| > 171.2 | 100 | 79.41% to 100% | 12 | 5.637% to 21.56% | 1.136 |
| > 172.2 | 100 | 79.41% to 100% | 13.33 | 6.583% to 23.16% | 1.154 |
| > 173.4 | 100 | 79.41% to 100% | 14.67 | 7.556% to 24.73% | 1.172 |
| > 175.6 | 100 | 79.41% to 100% | 16 | 8.55% to 26.28% | 1.19 |
| > 178.5 | 100 | 79.41% to 100% | 17.33 | 9.565% to 27.81% | 1.21 |
| > 180.9 | 100 | 79.41% to 100% | 18.67 | 10.6% to 29.33% | 1.23 |
| > 182.1 | 100 | 79.41% to 100% | 20 | 11.65% to 30.83% | 1.25 |
| > 183.3 | 100 | 79.41% to 100% | 21.33 | 12.71% to 32.32% | 1.271 |
| > 184.4 | 100 | 79.41% to 100% | 22.67 | 13.79% to 33.79% | 1.293 |
| > 184.9 | 100 | 79.41% to 100% | 24 | 14.89% to 35.25% | 1.316 |
| > 186.1 | 100 | 79.41% to 100% | 25.33 | 15.99% to 36.7% | 1.339 |
| > 188.9 | 100 | 79.41% to 100% | 26.67 | 17.11% to 38.14% | 1.364 |
| > 191.1 | 100 | 79.41% to 100% | 28 | 18.24% to 39.56% | 1.389 |
| > 191.9 | 100 | 79.41% to 100% | 29.33 | 19.38% to 40.98% | 1.415 |
| > 193.1 | 100 | 79.41% to 100% | 30.67 | 20.53% to 42.38% | 1.442 |
| > 195.2 | 100 | 79.41% to 100% | 32 | 21.69% to 43.78% | 1.471 |
| > 196.6 | 100 | 79.41% to 100% | 33.33 | 22.86% to 45.17% | 1.5 |
| > 197.2 | 100 | 79.41% to 100% | 34.67 | 24.04% to 46.54% | 1.531 |
| > 198.7 | 100 | 79.41% to 100% | 36 | 25.23% to 47.91% | 1.563 |
| > 204.8 | 100 | 79.41% to 100% | 37.33 | 26.43% to 49.27% | 1.596 |
| > 210 | 100 | 79.41% to 100% | 38.67 | 27.64% to 50.62% | 1.63 |
| > 211.1 | 100 | 79.41% to 100% | 40 | 28.85% to 51.96% | 1.667 |
| > 214.5 | 100 | 79.41% to 100% | 41.33 | 30.08% to 53.3% | 1.705 |
| > 219.2 | 100 | 79.41% to 100% | 42.67 | 31.31% to 54.62% | 1.744 |
| > 224.5 | 100 | 79.41% to 100% | 44 | 32.55% to 55.94% | 1.786 |
| > 228.8 | 100 | 79.41% to 100% | 45.33 | 33.79% to 57.25% | 1.829 |
| > 230.8 | 100 | 79.41% to 100% | 46.67 | 35.05% to 58.55% | 1.875 |
| > 231.7 | 100 | 79.41% to 100% | 48 | 36.31% to 59.85% | 1.923 |
| > 232.9 | 100 | 79.41% to 100% | 49.33 | 37.58% to 61.14% | 1.974 |
| > 238.2 | 100 | 79.41% to 100% | 50.67 | 38.86% to 62.42% | 2.027 |
| > 243.5 | 100 | 79.41% to 100% | 52 | 40.15% to 63.69% | 2.083 |
| > 244.7 | 100 | 79.41% to 100% | 53.33 | 41.45% to 64.95% | 2.143 |
| > 247.5 | 100 | 79.41% to 100% | 54.67 | 42.75% to 66.21% | 2.206 |
| > 250.4 | 100 | 79.41% to 100% | 56 | 44.06% to 67.45% | 2.273 |
| > 251.6 | 100 | 79.41% to 100% | 57.33 | 45.38% to 68.69% | 2.344 |
| > 252.4 | 100 | 79.41% to 100% | 58.67 | 46.7% to 69.92% | 2.419 |
| > 254.2 | 100 | 79.41% to 100% | 60 | 48.04% to 71.15% | 2.5 |
| > 257.2 | 100 | 79.41% to 100% | 61.33 | 49.38% to 72.36% | 2.586 |
| > 259 | 100 | 79.41% to 100% | 62.67 | 50.73% to 73.57% | 2.679 |
| > 260.8 | 100 | 79.41% to 100% | 64 | 52.09% to 74.77% | 2.778 |
| > 263.3 | 100 | 79.41% to 100% | 65.33 | 53.46% to 75.96% | 2.885 |
| > 268.8 | 100 | 79.41% to 100% | 66.67 | 54.83% to 77.14% | 3 |
| > 277.6 | 100 | 79.41% to 100% | 68 | 56.22% to 78.31% | 3.125 |
| > 282.9 | 100 | 79.41% to 100% | 69.33 | 57.62% to 79.47% | 3.261 |
| > 283.9 | 100 | 79.41% to 100% | 70.67 | 59.02% to 80.62% | 3.409 |
| > 286.3 | 100 | 79.41% to 100% | 72 | 60.44% to 81.76% | 3.571 |
| > 289.3 | 100 | 79.41% to 100% | 73.33 | 61.86% to 82.89% | 3.75 |
| > 290.4 | 100 | 79.41% to 100% | 74.67 | 63.3% to 84.01% | 3.947 |
| > 294.7 | 100 | 79.41% to 100% | 76 | 64.75% to 85.11% | 4.167 |
| > 300.8 | 100 | 79.41% to 100% | 77.33 | 66.21% to 86.21% | 4.412 |
| > 304 | 100 | 79.41% to 100% | 78.67 | 67.68% to 87.29% | 4.688 |
| > 310.4 | 100 | 79.41% to 100% | 80 | 69.17% to 88.35% | 5 |
| > 319.3 | 100 | 79.41% to 100% | 81.33 | 70.67% to 89.4% | 5.357 |
| > 324.4 | 100 | 79.41% to 100% | 82.67 | 72.19% to 90.43% | 5.769 |
| > 326.2 | 100 | 79.41% to 100% | 84 | 73.72% to 91.45% | 6.25 |
| > 328.7 | 100 | 79.41% to 100% | 85.33 | 75.27% to 92.44% | 6.818 |
| > 341.4 | 100 | 79.41% to 100% | 86.67 | 76.84% to 93.42% | 7.5 |
| > 353.1 | 100 | 79.41% to 100% | 88 | 78.44% to 94.36% | 8.333 |
| > 367.7 | 100 | 79.41% to 100% | 89.33 | 80.06% to 95.28% | 9.375 |
| > 381.9 | 100 | 79.41% to 100% | 90.67 | 81.71% to 96.16% | 10.71 |
| > 383.7 | 100 | 79.41% to 100% | 92 | 83.4% to 97.01% | 12.5 |
| > 391.7 | 100 | 79.41% to 100% | 93.33 | 85.12% to 97.8% | 15 |
| > 400.4 | 100 | 79.41% to 100% | 94.67 | 86.9% to 98.53% | 18.75 |
| > 404.8 | 100 | 79.41% to 100% | 96 | 88.75% to 99.17% | 25 |
| > 421.9 | 93.75 | 69.77% to 99.84% | 96 | 88.75% to 99.17% | 23.44 |
| > 437.2 | 93.75 | 69.77% to 99.84% | 97.33 | 90.7% to 99.68% | 35.16 |
| > 448 | 93.75 | 69.77% to 99.84% | 98.67 | 92.79% to 99.97% | 70.31 |
| > 547.1 | 93.75 | 69.77% to 99.84% | 100 | 95.2% to 100% | |
| > 646.2 | 87.5 | 61.65% to 98.45% | 100 | 95.2% to 100% | |
| > 689 | 81.25 | 54.35% to 95.95% | 100 | 95.2% to 100% | |
| > 733.3 | 75 | 47.62% to 92.73% | 100 | 95.2% to 100% | |
| > 755.6 | 68.75 | 41.34% to 88.98% | 100 | 95.2% to 100% | |
| > 769 | 62.5 | 35.43% to 84.8% | 100 | 95.2% to 100% | |
| > 791.5 | 56.25 | 29.88% to 80.25% | 100 | 95.2% to 100% | |
| > 818.2 | 50 | 24.65% to 75.35% | 100 | 95.2% to 100% | |
| > 901 | 43.75 | 19.75% to 70.12% | 100 | 95.2% to 100% | |
| > 1069 | 37.5 | 15.2% to 64.57% | 100 | 95.2% to 100% | |
| > 1356 | 31.25 | 11.02% to 58.66% | 100 | 95.2% to 100% | |
| > 1572 | 25 | 7.266% to 52.38% | 100 | 95.2% to 100% | |
| > 1621 | 18.75 | 4.047% to 45.65% | 100 | 95.2% to 100% | |
| > 1692 | 12.5 | 1.551% to 38.35% | 100 | 95.2% to 100% | |
| > 1814 | 6.25 | 0.1581% to 30.23% | 100 | 95.2% to 100% | |

TABLE 9

Cut-off values for ASC levels in serum-derived extracellular vesicles (EVs) for Stroke

| Cutoff (pg/ml) | Sensitivity% | 95% CI | Specificity% | 95% CI | Likelihood ratio |
|---|---|---|---|---|---|
| > 28.56 | 100 | 79.41% to 100% | 6.25 | 0.1581% to 30.23% | 1.067 |
| > 31.31 | 100 | 79.41% to 100% | 12.5 | 1.551% to 38.35% | 1.143 |
| > 33.88 | 100 | 79.41% to 100% | 18.75 | 4.047% to 45.65% | 1.231 |
| > 37.46 | 100 | 79.41% to 100% | 25 | 7.266% to 52.38% | 1.333 |
| > 41.38 | 100 | 79.41% to 100% | 31.25 | 11.02% to 58.66% | 1.455 |
| > 44.01 | 100 | 79.41% to 100% | 37.5 | 15.2% to 64.57% | 1.6 |
| > 44.38 | 100 | 79.41% to 100% | 43.75 | 19.75% to 70.12% | 1.778 |
| > 45.13 | 100 | 79.41% to 100% | 50 | 24.65% to 75.35% | 2 |
| > 46.71 | 100 | 79.41% to 100% | 56.25 | 29.88% to 80.25% | 2.286 |
| > 48.51 | 100 | 79.41% to 100% | 62.5 | 35.43% to 84.8% | 2.667 |
| > 49.35 | 100 | 79.41% to 100% | 68.75 | 41.34% to 88.98% | 3.2 |
| > 51.09 | 100 | 79.41% to 100% | 75 | 47.62% to 92.73% | 4 |
| > 58.1 | 100 | 79.41% to 100% | 81.25 | 54.35% to 95.95% | 5.333 |
| > 69.76 | 100 | 79.41% to 100% | 87.5 | 61.65% to 98.45% | 8 |
| > 81.6 | 100 | 79.41% to 100% | 93.75 | 69.77% to 99.84% | 16 |
| > 97.57 | 100 | 79.41% to 100% | 100 | 79.41% to 100% | |
| > 114.9 | 93.75 | 69.77% to 99.84% | 100 | 79.41% to 100% | |
| > 130.2 | 87.5 | 61.65% to 98.45% | 100 | 79.41% to 100% | |
| > 138.7 | 81.25 | 54.35% to 95.95% | 100 | 79.41% to 100% | |
| > 139 | 75 | 47.62% to 92.73% | 100 | 79.41% to 100% | |
| > 143.6 | 68.75 | 41.34% to 88.98% | 100 | 79.41% to 100% | |
| > 153.2 | 62.5 | 35.43% to 84.8% | 100 | 79.41% to 100% | |
| > 165.6 | 56.25 | 29.88% to 80.25% | 100 | 79.41% to 100% | |
| > 202.6 | 50 | 24.65% to 75.35% | 100 | 79.41% to 100% | |
| > 261.5 | 43.75 | 19.75% to 70.12% | 100 | 79.41% to 100% | |
| > 292.9 | 37.5 | 15.2% to 64.57% | 100 | 79.41% to 100% | |
| > 361.4 | 31.25 | 11.02% to 58.66% | 100 | 79.41% to 100% | |
| > 441.3 | 25 | 7.266% to 52.38% | 100 | 79.41% to 100% | |
| > 459.4 | 18.75 | 4.047% to 45.65% | 100 | 79.41% to 100% | |
| > 465.8 | 12.5 | 1.551% to 38.35% | 100 | 79.41% to 100% | |
| > 493.5 | 6.25 | 0.1581% to 30.23% | 100 | 79.41% to 100% | |

### Example 3: Examination of Inflammasome Proteins as Biomarkers of Traumatic Brain Injury (TBI)

As defined by the US Center for Disease Control (“CDC), a traumatic brain injury (“TBF”) is “a disruption in the normal function of the brain that can be caused by a bump, blow, or jolt to the head, or penetrating head injury.” Important to the care of patients with TBI is the need for biomarkers that can predict onset, exacerbation as well as response to treatment. Additionally, there is a need for a minimally invasive method of harvesting these biomarkers for analysis.

The inflammasome is a key mediator of the innate immune response that in the CNS was first described to mediate inflammation after spinal cord injury². The inflammasome is a multiprotein complex involved in the activation of caspase-1 and the processing of the pro-inflammatory cytokines IL-10 and IL-18³.

In this example, the expression level of inflammasome proteins in serum samples from patients with TBI are determined. Further, an examination of the sensitivity and specificity of inflammasome signaling proteins as biomarkers of TBI was examined.

### Materials and Methods

### Participants

In this study, serum samples were analyzed from 120 normal donors and 21 patients that were diagnosed with TBI. Samples were purchased from BioreclamationlVT: The normal donor group consisted of samples obtained from 60 male and 60 female donors in the age range of 20 to 70 years old. The age range in the TBI group consisted of samples obtained from patients in the age range of 24 to 64 years old. Additionally, twenty-one control cerebral spinal fluid (“CSF”) samples were obtained from BioreclamationIVT, 9 CSF samples were obtained from the cohort of patients.

### Protein Assay

Concentration of inflammasome proteins ASC, IL-1β and IL-18 in serum and CSF was analyzed using a Simple Plex and a Simple Plex Explorer software. Results shown correspond to the mean of each sample run in triplicates. It should be noted that any system/instrument known in the art can be used to measure the levels of proteins (e.g., inflammasome proteins) in bodily fluids. Samples were collected three times a day for the first 5 days since patients arrived to the hospital. Samples were analyzed for the 1st, 2nd collection (Day 1) as well as 4th and 6th collections (Day 2)

### Biomarker Analyses

Prism 7 software (GraphPad) was used to analyze the data obtained from the Simple Plex Explorer Software. Comparisons between groups were carried after identifying outliers followed by determination of the area under the receiver operator characteristic (ROC) curve, as well as the 95% confidence interval (CI). The p-value of significance used was <0.05. Sensitivity and specificity of each biomarker was obtained for a range of different cut-off points. Samples that yielded a protein value below the level of detection of the assay were not included in the analyses for that analyte.

ROC curves are summarized as the area under the curve (AUC). A perfect AUC value is 1.0, where 100% of subjects in the population will be correctly classified as having TBI or not. In contrast, an AUC of 0.5 signifies that subjects are randomly classified as either positive or negative for TBI, which has no clinical utility. It has been suggested that an AUC between 0.9 to 1.0 applies to an excellent biomarker; from 0.8 to 0.9, good; 0.7 to 0.8 fair; 0.6 to 0.7, poor and 0.5 to 0.6, fail.⁵

### Results

### Caspase-1 and ASC are Elevated in the Serum of Patients After TBI

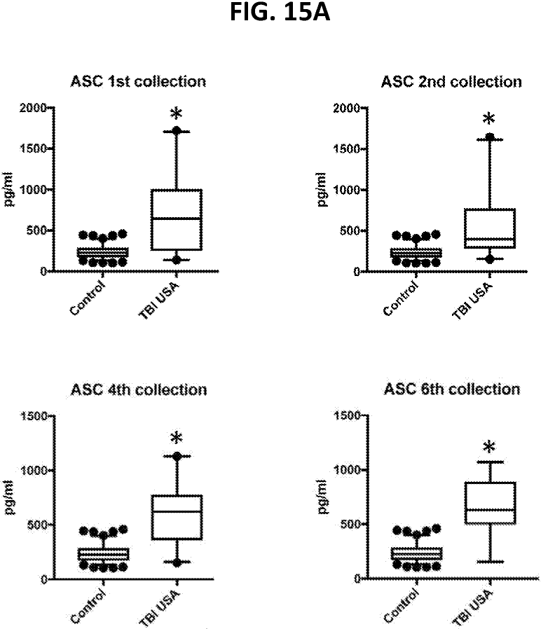

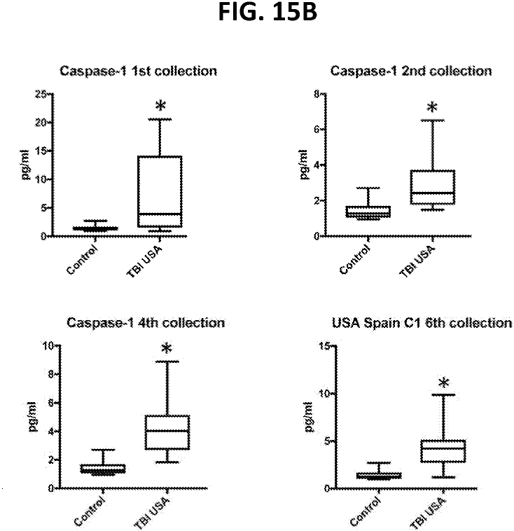

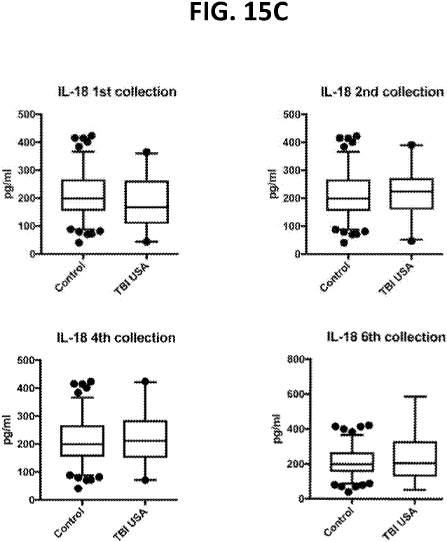

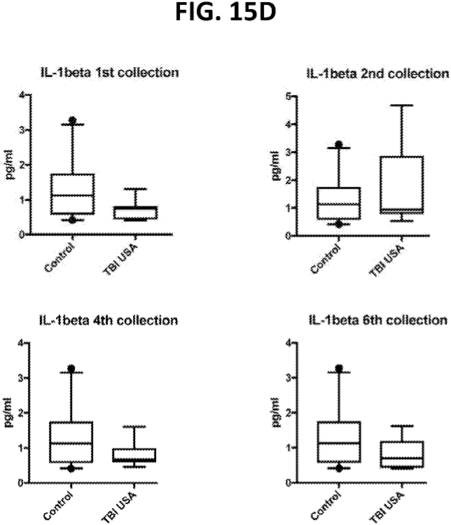

Serum samples from TBI patients were analyzed and compared to serum from healthy/control individuals using a Simple Plex assay (Protein Simple) for the protein expression of the inflammasome signaling proteins caspase-1, ASC, IL-1β and IL-18 (FIGS. 15A-15D). The protein levels of caspase-1, ASC and IL-18 in the serum of TBI patients was higher than in the control group. However, the levels of IL-10 were lower in the TBI than controls.

### ASC and Caspase-1 are Good Serum Biomarkers of TBI

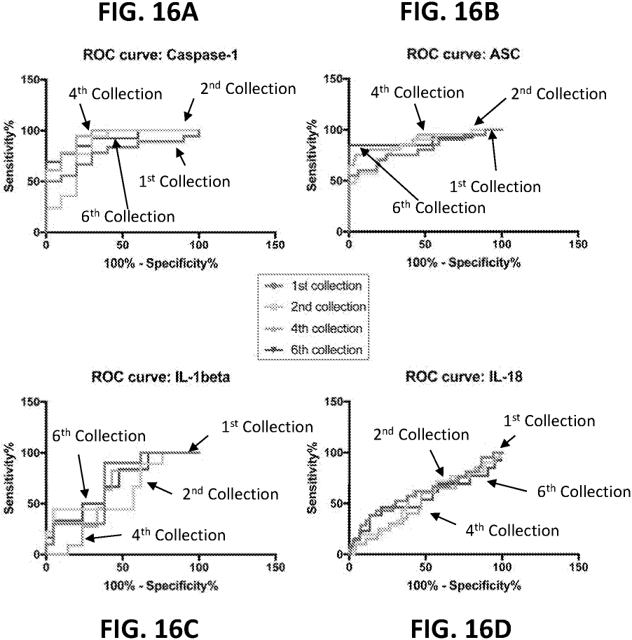

To then determine if these inflammasome signaling proteins have the potential to be reliable biomarkers for TBI pathology, the area under the curve (AUC) for caspase-1, ASC, EL-10 and IL-18 (FIGS. 16A-D) were determined. Of the proteins measured, caspase-1 and ASC were shown to be the best biomarkers (FIGS. 16A and B) with an AUC of 0.93 (4^{th} collection) and 0.90 (6^{th} collection), respectively (Tables 10A-10D).

Table 10A-D: ROC analysis results for inflammasome signaling proteins Caspase-1 (Table 10A), ASC (Table 10B), IL-10 (Table 10C) and IL-18 (Table 10D) in serum including area, standard error (STD. ERROR), 95% confidence interval (CI) and p-value for collections 1^{st}, 2^{nd}, 4^{th} and 6^{th}.

TABLE 10A

ROC analysis for Caspase-1 in Serum

| BIOMARKER | AUC | STD. ERROR | 95% C.I. | P VALUE |
|---|---|---|---|---|
| 1^{st} Collection | 0.78 | 0.08772 | 0.6058 to 0.9497 | 0.01 |
| 2^{nd} Collection | 0.83 | 0.0479 | 0.8395 to 1.027 | 0.005 |
| 4^{th} Collection | 0.93 | 0.1407 | 0.8353 to 0.8869 | 0.0002 |
| 6^{th} Collection | 0.91 | 0.06065 | 0.7888 to 1.027 | 0.001 |

TABLE 10B

ROC analysis for ASC in Serum

| BIOMARKER | AUC | STD. ERROR | 95% C.I. | P VALUE |
|---|---|---|---|---|
| 1^{st} Collection | 0.80 | 0.06472 | 0.6762 to 0.9299 | <0.0001 |
| 2^{nd} Collection | 0.84 | 0.05026 | 0.7425 to 0.9395 | <0.0001 |
| 4^{th} Collection | 0.89 | 0.04898 | 0.7931 to 0.9851 | <0.0001 |
| 6^{th} Collection | 0.90 | 0.0697 | 0.759 to 1.032 | <0.0001 |

TABLE 10C

ROC analysis for IIL-1 in Serum

| BIOMARKER | AUC | STD. ERROR | 95% C.I. | P VALUE |
|---|---|---|---|---|
| 1^{st} Collection | 0.7 | 0.0965 | 0.5109 to 0.8891 | 0.0759 |
| 2^{nd} Collection | 0.64 | 0.1182 | 0.4085 to 0.8719 | 0.2304 |
| 4^{th} Collection | 0.6234 | 0.09765 | 0.432 to 0.8148 | 0.2582 |
| 6^{th} Collection | 0.6984 | 0.1162 | 0.4707 to 0.9261 | 0.1448 |

TABLE 10D

ROC analysis for IL-18 in Serum

| BIOMARKER | AUC | STD. ERROR | 95% C.I. | P VALUE |
|---|---|---|---|---|
| 1^{st} Collection | 0.61 | 0.07475 | 0.4593 to 0.7524 | 0.1227 |
| 2^{nd} Collection | 0.55 | 0.07064 | 0.4082 to 0.6851 | 0.4966 |
| 4^{th} Collection | 0.51 | 0.0713 | 0.372 to 0.6515 | 0.8666 |
| 6^{th} Collection | 0.55 | 0.1015 | 0.3532 to 0.7509 | 0.5387 |

Furthermore, the cut-off point for caspase-1 was 1.943 pg/ml with 94% sensitivity and 89% specificity (Table 11A). For ASC, the cut-off point was 451.3 pg/ml with 85% sensitivity and 99% specificity (Table 11B). Moreover, we found that in regards to caspase-1 for 100% sensitivity, the cut-off point was 1.679 pg/ml with 78% specificity. For ASC, the cut-off point was 153.4 pg/ml and a 19% specificity (see Table 16 (4^{th} collection)). In the case of caspase-1, for 100% specificity, the cut-off point was 2.717 pg/ml with 78% sensitivity (see Table 15 (4^{th} collection)). For ASC with 100% specificity, the cut-off point was 462.4 pg/ml with 85% sensitivity (see Table 16 (4^{th} collection)). Thus, these findings indicate that caspase-1 and ASC are reliable serum biomarkers for TBI.

Table 11A-B: ROC analysis results for caspase-1 (Table 11A) and ASC (Table 11B) in serum including cut-off point in pg/ml, sensitivity and specificity, as well as positive and negative likelihood ratios (LR+/LR-).

TABLE 11A

ROC analysis for Caspase-1 in Serum

| Biomarker | Cut-off point (pg/ml) | Sensitivity (%) | Specificity (%) | LR + | LR- |
|---|---|---|---|---|---|
| 1^{st} Collection | > 1.439 | 83 | 67 | 2.50 | 0.25 |
| 2^{nd} Collection | > 1.531 | 94 | 78 | 4.24 | 0.08 |
| 4^{th} Collection | > 1.943 | 94 | 89 | 8.50 | 0.06 |
| 6^{th} Collection | > 1.947 | 85 | 89 | 7.62 | 0.17 |

TABLE 11B

ROC analysis for ASC in Serum

| Biomarker | Cut-off point (pg/ml) | Sensitivity (%) | Specificity (%) | LR + | LR- |
|---|---|---|---|---|---|
| 1^{st} Collection | > 210 | 85 | 43 | 1.50 | 0.35 |
| 2^{nd} Collection | > 275 | 81 | 72 | 2.91 | 0.26 |
| 4^{th} Collection | > 339.4 | 80 | 88 | 6.57 | 0.23 |
| 6^{th} Collection | > 451.3 | 85 | 99 | 97.26 | 0.16 |

### ASC is Elevated in the Serum of Patients With Unfavorable Outcomes After TBI

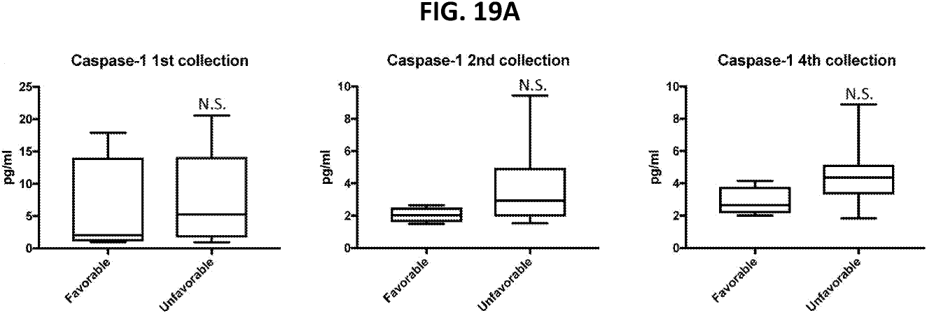

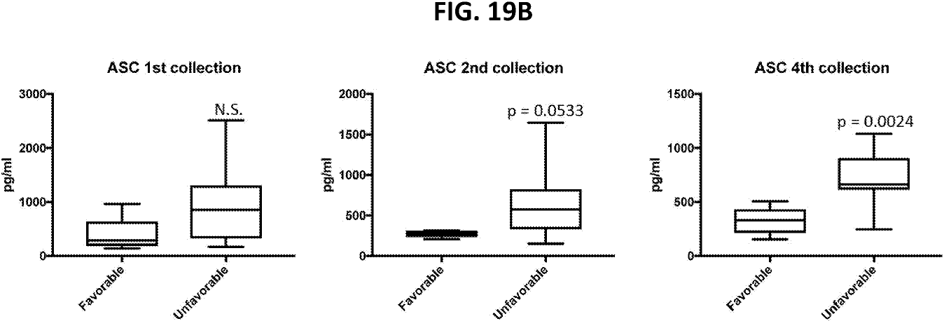

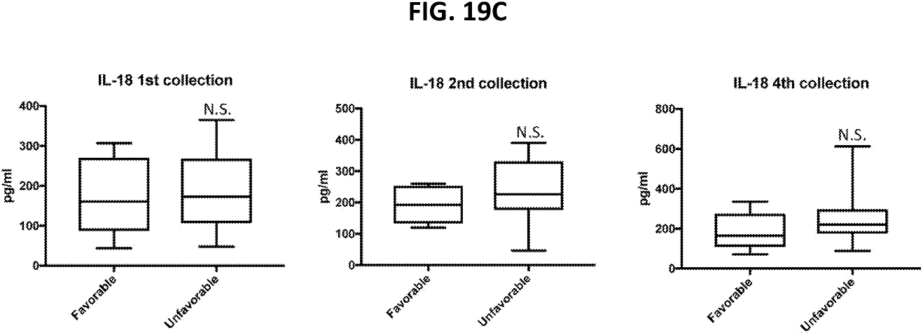

TBI patients were separated according to their clinical outcomes; either favorable or unfavorable outcomes based on the Glasgow Outcome Scale-Extended (GOSE) in which patients with a score of 6 to 8 were considered to have favorable outcomes and those with a score of 1 to 4 were considered to have unfavorable outcomes (Table s 12A and 12B). It was found that the protein level of ASC was higher in the serum of TBI patients with unfavorable outcomes when compared to the samples obtained from patients with favorable outcomes (FIG. 19B), whereas the caspase-1 (FIG. 19A) and IL-18 (FIG. 19C) levels were not statistically different between the two groups.

### ASC is a Good Prognostic Biomarker of TBI in Serum

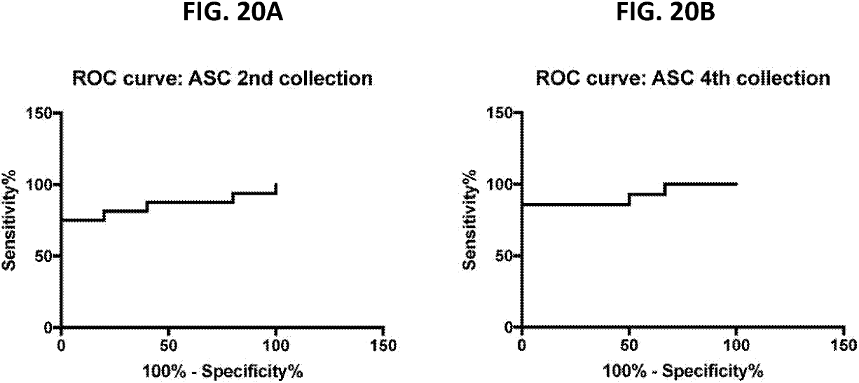

To determine if ASC can be used as prognostic biomarkers of TBI, we determined the AUC for ASC at the 2nd (FIG. 20A) and 4th collection (FIG. 20B). The AUC for ASC was 0.9167 in the 4th collection with a CI between 0.7914 and 1.042 (Table 12A). Furthermore, the cut-off point was 547.6 pg/ml with 86% sensitivity and 100% specificity (Table 12B and Table 19 (4^{th} collection). Thus, these findings indicated that ASC is a promising prognostic biomarker of TBI in serum.

Table 12A-B: ROC analysis results for ASC in serum for Favorable (Table 12A) vs Unfavorable (Table 12B) outcomes, including area, standard error (STD. ERROR), 95% confidence interval (CI), p-value (see Table 12A), cut-off point in pg/ml, sensitivity and specificity, as well as positive and negative likelihood ratios (LR+/LR-) (see Table 12B) for collections 1^{st}, 2^{nd} and 4^{th}.

TABLE 12A

ROC analysis for ASC in Serum (GOSE) for favorable outcome

| BIOMARKER | AREA | STD. ERROR | 95% C.I. | P VALUE |
|---|---|---|---|---|
| 1^{st} Collection | 0.7625 | 0.1133 | 0.544 to 0.9846 | 0.0829 |
| 2^{nd} Collection | 0.85 | 0.08355 | 0.6862 to 1.014 | 0.0208 |
| 4^{th} Collection | 0.9167 | 0.06391 | 0.7914 to 1.042 | 0.0039 |

TABLE 12B

ROC analysis for ASC in Serum (GOSE) for unfavorable outcome

| BIOMARKER | CUT-OFF POINT (pg/ml) | SENSITIVI TY (%) | SPECIFICI TY (%) | LR + | LR- |
|---|---|---|---|---|---|
| 1^{st} Collection | > 353.7 | 75 | 80 | 3.75 | 0.31 |
| 2^{nd} Collection | > 311.2 | 81.25 | 80 | 4.06 | 0.23 |
| 4^{th} Collection | > 547.6 | 85.71 | 100 | | 0.14 |

### ASC and IL-18 are Elevated in the CSF of Patients After TBI

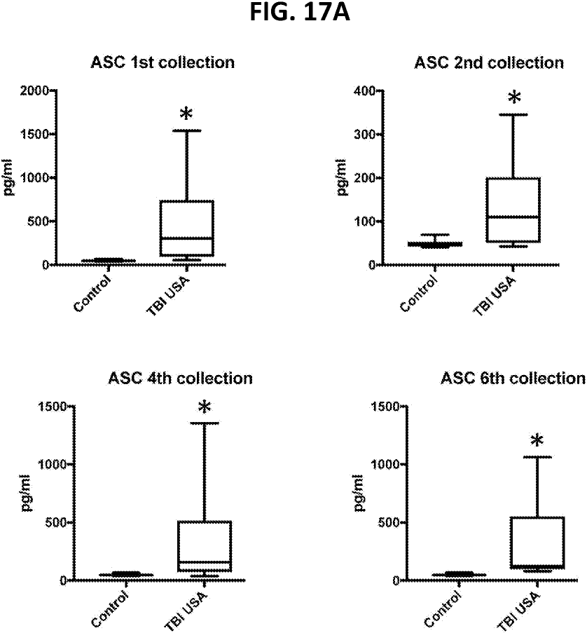

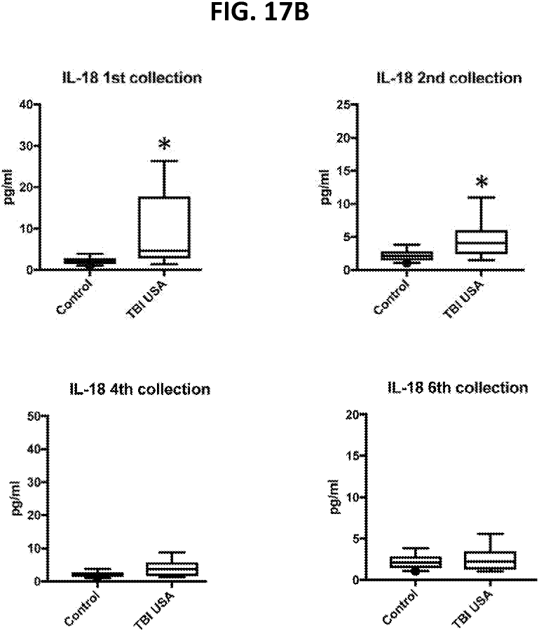

CSF samples from TBI patients were analyzed and compared to CSF from healthy/control individuals using a Simple Plex assay (Protein Simple) for the protein expression of the inflammasome signaling proteins ASC and IL-18 (FIGS. 17A and 17B). The protein levels of ASC and IL-18 in the serum of TBI patients were both higher than in the control group.

### ASC and IL-18 are Good CSF Biomarkers of TBI

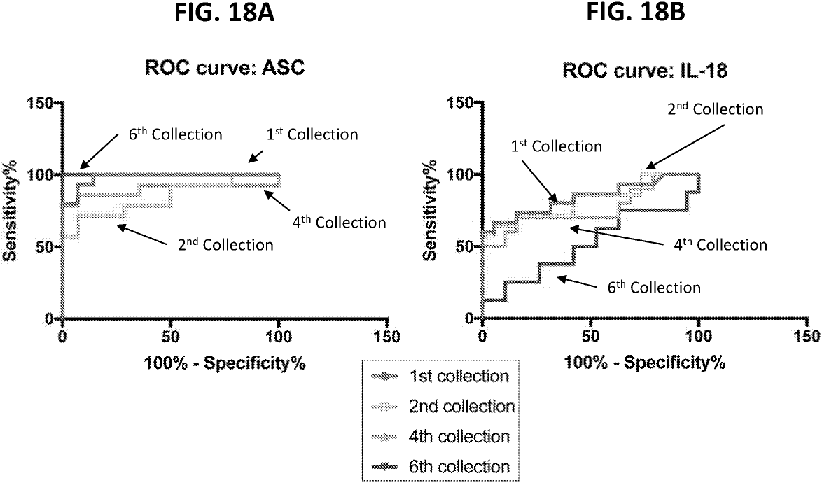

To then determine if these inflammasome signaling proteins have the potential to be reliable biomarkers for TBI pathology, the area under the curve (AUC) for ASC, and IL-18 (FIGS. 18A and 18B) in CSF were determined. ASC and IL-18 were shown to be the best biomarkers (FIGS. 18A and 18B) with an AUC of 1.0 (6^{th} collection) and 0.84 (1^{st} collection), respectively (Tables 13A and 13B).

Tables 13A and 13B: ROC analysis results for ASC (Table 13A) and IL-18 (Table 13B) in CSF including cut-off point in pg/ml, sensitivity and specificity, as well as positive and negative likelihood ratios (LR+/LR-).

TABLE 13A

ROC analysis of ASC in CSF

| BIOMARKER | AUC | STD. ERROR | 95% C.I. | P VALUE |
|---|---|---|---|---|
| 1^{st} Collection | 0.981 | 0.0195 | 0.9427 to 1.019 | <0.0001 |
| 2^{nd} Collection | 0.8418 | 0.07661 | 0.6917 to 0.992 | 0.0021 |
| 4^{th} Collection | 0.898 | 0.07262 | 0.7556 to 1.04 | 0.0003 |
| 6^{th} Collection | 1 | 0 | 1 to 1 | 0.0001 |

TABLE 13B

ROC analysis of IL-18 in CSF

| BIOMARKER | AUC | STD. ERROR | 95% C.I. | P VALUE |
|---|---|---|---|---|
| 1^{st} Collection | 0.8404 | 0.0731 | 0.6971 to 0.9836 | 0.0008 |
| 2^{nd} Collection | 0.8195 | 0.07969 | 0.6634 to 0.9757 | 0.002 |
| 4^{th} Collection | 0.7632 | 0.1061 | 0.552 to 0.9711 | 0.9711 |
| 6^{th} Collection | 0.5132 | 0.1344 | 0.2498 to 0.7765 | 0.9154 |

Furthermore, the cut-off point for ASC, the cut-off point was 74.33 pg/ml with 100% sensitivity and 100% specificity (Table 14A and Table 17). For IL-18, the cut-off point was 2.722 pg/ml with 80% sensitivity and 68% specificity (Table 14B and Table 18). As shown in Table 18, in the case of IL-18, for 100% specificity, the cut-off point was 3.879 pg/ml with 60% sensitivity; for 100% sensitivity, the cut-off point was 1.358 pg/ml, with 16%specificity. Thus, these findings indicate that ASC and IL-18 are reliable serum biomarkers for TBI.

Table 14A-B: ROC analysis results for ASC (Table 14A) and IL-18 (Table 14B) in CSF including cut-off point in pg/ml, sensitivity and specificity, as well as positive and negative likelihood ratios (LR+/LR-).

TABLE 14A

ROC analysis for ASC in CSF

| Biomarker | Cut-off point (pg/ml) | Sensitivity (%) | Specificity (%) | LR + | LR- |
|---|---|---|---|---|---|
| 1^{st} Collection | > 55.11 | 100 | 85.71 | 7 | 0 |
| 2^{nd} Collection | > 50.25 | 78.57 | 64.29 | 2.20 | 0.33 |
| 4^{th} Collection | > 64.58 | 85.71 | 92.86 | 12 | 0.15 |
| 6^{th} Collection | > 74.33 | 100 | 100 | | 0 |

TABLE 14B

ROC analysis for IL-18 in CSF

| Biomarker | Cut-off point (pg/ml) | Sensitivity (%) | Specificity (%) | LR + | LR- |
|---|---|---|---|---|---|
| 1^{st} Collection | > 2.722 | 80 | 68.42 | 2.53 | 0.29 |
| 2^{nd} Collection | > 2.221 | 85.71 | 57.89 | 2.04 | 0.25 |
| 4^{th} Collection | > 3.055 | 70 | 84.21 | 4.43 | 0.36 |
| 6^{th} Collection | > 1.707 | 75 | 36.84 | 1.19 | 0.68 |

### Conclusions:

In this study, a statistically significant higher level of ASC and caspase-1 was detected in the serum of TBI patients when compared to healthy subjects. In this study, we show that ASC and IL-18 are reliable biomarkers for TBI in CSF with AUC values of 1.0 and 0.84, respectively. Most importantly, since obtaining CSF is a very invasive procedure, then our findings on serum are even more applicable to the typical clinical setting. Accordingly, we found that the AUC values for ASC was 0.90 and for caspase-1, 0.93. Thus caspase-1 and ASC should be considered as biomarkers in the care of patients with brain injury.

Moreover, the data showed that when comparing patients with unfavorable outcomes to patients with favorable outcomes chronically after TBI, the AUC for ASC was 0.92; thus, highlighting the usefulness of ASC as a TBI biomarker in serum, and, in this case, as a predictive biomarker of brain injury.

Thus, based on these findings ASC and caspace-1 are both promising biomarkers with a high AUC value, a high sensitivity and high specificity in serum. Additionally, based on these findings, ASC and IL-18 are both promising biomarkers with a high AUC value, a high sensitivity and high specificity in CSF. Importantly, ASC as a biomarker for TBI with other diagnostic criteria may further increase the sensitivity of ASC as a biomarker for TBI beyond what is described in this example.

Importantly, in this study, ASC has been identified as a potential biomarker of TBI pathology with a high AUC value of 0.9448 and with sensitivities above 80% and a specificity of over 90%.

### Incorporation by Reference

The following references are incorporated by reference in their entireties for all purposes.

1. Adamczak, S., Dale, G., De Rivero Vaccari, J.P., Bullock, M.R., Dietrich, W.D., and Keane, R.W.(2012). Inflammasome proteins in cerebrospinal fluid of brain-injured patients as biomarkers of functional outcome: clinical article. J Neurosurg 117, 1119-1125.

2. Brand, F.J., 3rd, Forouzandeh, M., Kaur, H., Travascio, F., and De Rivero Vaccari, J.P. (2016). Acidification changes affect the inflammasome in human nucleus pulposus cells. J Inflamm (Lond) 13, 29.

3. De Rivero Vaccari, J.P., Brand, F., 3rd, Adamczak, S., Lee, S.W., Perez-Barcena, J., Wang, M.Y., Bullock, M.R., Dietrich, W.D., and Keane, R.W. (2016). Exosome-mediated inflammasome signaling after central nervous system injury. J Neurochem 136 Suppl 1, 39-48.

4. Keane, R.W., Dietrich, W.D., and De Rivero Vaccari, J.P. (2018). Inflammasome Proteins As Biomarkers of Multiple Sclerosis. Front Neurol 9,135.

5. Xia J, Broadhurst DI, Wilson M and Wishart DS. Translational biomarker discovery in clinical metabolomics: an introductory tutorial. Metabolomics. 2013;9:280-299.

TABLE 15

Full ROC Data for caspase-1 4^{th} collection in serum

| Cutoff (pg/ml) | Sensitivity% | 95% CI | Specificity% | 95% CI | Likelihood ratio |
|---|---|---|---|---|---|
| > 0.984 | 100 | 81.47% to 100% | 11.11 | 0.2809% to 48.25% | 1.125 |
| > 1.048 | 100 | 81.47% to 100% | 22.22 | 2.814% to 60.01% | 1.286 |
| > 1.091 | 100 | 81.47% to 100% | 33.33 | 7.485% to 70.07% | 1.5 |
| > 1.19 | 100 | 81.47% to 100% | 44.44 | 13.7% to 78.8% | 1.8 |
| > 1.338 | 100 | 81.47% to 100% | 55.56 | 21.2% to 86.3% | 2.25 |
| > 1.461 | 100 | 81.47% to 100% | 66.67 | 29.93% to 92.51% | 3 |
| > 1.679 | 100 | 81.47% to 100% | 77.78 | 39.99% to 97.19% | 4.5 |
| > 1.853 | 94.44 | 72.71% to 99.86% | 77.78 | 39.99% to 97.19% | 4.25 |
| > 1.943 | 94.44 | 72.71% to 99.86% | 88.89 | 51.75% to 99.72% | 8.5 |
| > 2.293 | 88.89 | 65.29% to 98.62% | 88.89 | 51.75% to 99.72% | 8 |
| > 2.577 | 83.33 | 58.58% to 96.42% | 88.89 | 51.75% to 99.72% | 7.5 |
| > 2.643 | 77.78 | 52.36% to 93.59% | 88.89 | 51.75% to 99.72% | 7 |
| > 2.717 | 77.78 | 52.36% to 93.59% | 100 | 66.37% to 100% | |
| > 2.812 | 72.22 | 46.52% to 90.31% | 100 | 66.37% to 100% | |
| > 3.174 | 66.67 | 40.99% to 86.66% | 100 | 66.37% to 100% | |
| > 3.68 | 61.11 | 35.75% to 82.7% | 100 | 66.37% to 100% | |
| > 3.947 | 55.56 | 30.76% to 78.47% | 100 | 66.37% to 100% | |
| > 4.027 | 50 | 26.02% to 73.98% | 100 | 66.37% to 100% | |
| > 4.105 | 44.44 | 21.53% to 69.24% | 100 | 66.37% to 100% | |
| > 4.397 | 38.89 | 17.3% to 64.25% | 100 | 66.37% to 100% | |
| > 4.71 | 33.33 | 13.34% to 59.01% | 100 | 66.37% to 100% | |
| > 4.95 | 27.78 | 9.695% to 53.48% | 100 | 66.37% to 100% | |
| > 5.139 | 22.22 | 6.409% to 47.64% | 100 | 66.37% to 100% | |
| > 5.157 | 16.67 | 3.579% to 41.42% | 100 | 66.37% to 100% | |
| > 5.59 | 11.11 | 1.375% to 34.71% | 100 | 66.37% to 100% | |
| > 7.452 | 5.556 | 0.1406% to 27.29% | 100 | 66.37% to 100% | |

TABLE 16

Full ROC Data for ASC 6^{th} collection in serum

| Cutoff (pg/ml) | Sensitivity% | 95% CI | Specificity% | 95% CI | Likelihood ratio |
|---|---|---|---|---|---|
| > 105.8 | 100 | 75.29% to 100% | 0.8696 | 0.02201% to 4.75% | 1.009 |
| > 107.9 | 100 | 75.29% to 100% | 1.739 | 0.2113% to 6.141% | 1.018 |
| > 112.1 | 100 | 75.29% to 100% | 2.609 | 0.5412% to 7.435% | 1.027 |
| > 123.3 | 100 | 75.29% to 100% | 3.478 | 0.9557% to 8.667% | 1.036 |
| > 132.4 | 100 | 75.29% to 100% | 4.348 | 1.427% to 9.855% | 1.045 |
| > 133 | 100 | 75.29% to 100% | 5.217 | 1.939% to 11.01% | 1.055 |
| > 134.2 | 100 | 75.29% to 100% | 6.087 | 2.482% to 12.14% | 1.065 |
| > 135.2 | 100 | 75.29% to 100% | 6.957 | 3.051% to 13.25% | 1.075 |
| > 135.5 | 100 | 75.29% to 100% | 7.826 | 3.641% to 14.34% | 1.085 |
| > 135.8 | 100 | 75.29% to 100% | 8.696 | 4.249% to 15.41% | 1.095 |
| > 136.1 | 100 | 75.29% to 100% | 9.565 | 4.872% to 16.47% | 1.106 |
| > 139.2 | 100 | 75.29% to 100% | 10.43 | 5.509% to 17.52% | 1.117 |
| > 142.6 | 100 | 75.29% to 100% | 11.3 | 6.158% to 18.55% | 1.127 |
| > 143.3 | 100 | 75.29% to 100% | 12.17 | 6.818% to 19.58% | 1.139 |
| > 144.6 | 100 | 75.29% to 100% | 13.04 | 7.488% to 20.6% | 1.15 |
| > 146.2 | 100 | 75.29% to 100% | 13.91 | 8.167% to 21.61% | 1.162 |
| > 147.5 | 100 | 75.29% to 100% | 14.78 | 8.854% to 22.61% | 1.173 |
| > 148.9 | 100 | 75.29% to 100% | 15.65 | 9.548% to 23.6% | 1.186 |
| > 150.4 | 100 | 75.29% to 100% | 16.52 | 10.25% to 24.59% | 1.198 |
| > 151.4 | 100 | 75.29% to 100% | 17.39 | 10.96% to 25.57% | 1.211 |
| > 151.8 | 100 | 75.29% to 100% | 18.26 | 11.67% to 26.55% | 1.223 |
| > 153.4 | 100 | 75.29% to 100% | 19.13 | 12.39% to 27.52% | 1.237 |
| > 155.5 | 92.31 | 63.97% to 99.81% | 19.13 | 12.39% to 27.52% | 1.141 |
| > 158.2 | 92.31 | 63.97% to 99.81% | 20 | 13.12% to 28.48% | 1.154 |
| > 160.8 | 92.31 | 63.97% to 99.81% | 20.87 | 13.85% to 29.44% | 1.167 |
| > 164 | 92.31 | 63.97% to 99.81% | 21.74 | 14.59% to 30.4% | 1.179 |
| > 168 | 92.31 | 63.97% to 99.81% | 22.61 | 15.33% to 31.35% | 1.193 |
| > 170.2 | 92.31 | 63.97% to 99.81% | 23.48 | 16.08% to 32.29% | 1.206 |
| > 171.2 | 92.31 | 63.97% to 99.81% | 24.35 | 16.83% to 33.23% | 1.22 |
| > 172.2 | 92.31 | 63.97% to 99.81% | 25.22 | 17.58% to 34.17% | 1.234 |
| > 173.4 | 92.31 | 63.97% to 99.81% | 26.09 | 18.34% to 35.1% | 1.249 |
| > 175.6 | 92.31 | 63.97% to 99.81% | 26.96 | 19.11% to 36.03% | 1.264 |
| > 178.5 | 92.31 | 63.97% to 99.81% | 27.83 | 19.87% to 36.95% | 1.279 |
| > 180.9 | 92.31 | 63.97% to 99.81% | 28.7 | 20.65% to 37.88% | 1.295 |
| > 182.1 | 92.31 | 63.97% to 99.81% | 29.57 | 21.42% to 38.79% | 1.311 |
| > 183.3 | 92.31 | 63.97% to 99.81% | 30.43 | 22.2% to 39.71% | 1.327 |
| > 184.4 | 92.31 | 63.97% to 99.81% | 31.3 | 22.98% to 40.62% | 1.344 |
| > 184.9 | 92.31 | 63.97% to 99.81% | 32.17 | 23.77% to 41.53% | 1.361 |
| > 185.7 | 92.31 | 63.97% to 99.81% | 33.04 | 24.56% to 42.43% | 1.379 |
| > 186.5 | 92.31 | 63.97% to 99.81% | 33.91 | 25.35% to 43.33% | 1.397 |
| > 188.9 | 92.31 | 63.97% to 99.81% | 34.78 | 26.14% to 44.23% | 1.415 |
| > 191.1 | 92.31 | 63.97% to 99.81% | 35.65 | 26.94% to 45.12% | 1.435 |
| > 191.9 | 92.31 | 63.97% to 99.81% | 36.52 | 27.74% to 46.01% | 1.454 |
| > 193.1 | 92.31 | 63.97% to 99.81% | 37.39 | 28.55% to 46.9% | 1.474 |
| > 195.2 | 92.31 | 63.97% to 99.81% | 38.26 | 29.35% to 47.79% | 1.495 |
| > 196.6 | 92.31 | 63.97% to 99.81% | 39.13 | 30.16% to 48.67% | 1.516 |
| > 197.2 | 92.31 | 63.97% to 99.81% | 40 | 30.98% to 49.55% | 1.538 |
| > 198.7 | 92.31 | 63.97% to 99.81% | 40.87 | 31.79% to 50.43% | 1.561 |
| > 202.1 | 92.31 | 63.97% to 99.81% | 41.74 | 32.61% to 51.3% | 1.584 |
| > 207.2 | 92.31 | 63.97% to 99.81% | 42.61 | 33.44% to 52.17% | 1.608 |
| > 210 | 92.31 | 63.97% to 99.81% | 43.48 | 34.26% to 53.04% | 1.633 |
| > 211.1 | 92.31 | 63.97% to 99.81% | 44.35 | 35.09% to 53.91% | 1.659 |
| > 213.9 | 92.31 | 63.97% to 99.81% | 45.22 | 35.92% to 54.77% | 1.685 |
| > 216.3 | 84.62 | 54.55% to 98.08% | 45.22 | 35.92% to 54.77% | 1.545 |
| > 216.8 | 84.62 | 54.55% to 98.08% | 46.09 | 36.75% to 55.63% | 1.569 |
| > 218.1 | 84.62 | 54.55% to 98.08% | 46.96 | 37.59% to 56.49% | 1.595 |
| > 220.4 | 84.62 | 54.55% to 98.08% | 47.83 | 38.43% to 57.34% | 1.622 |
| > 224.1 | 84.62 | 54.55% to 98.08% | 48.7 | 39.27% to 58.19% | 1.649 |
| > 227.1 | 84.62 | 54.55% to 98.08% | 49.57 | 40.11% to 59.04% | 1.678 |
| > 228.8 | 84.62 | 54.55% to 98.08% | 50.43 | 40.96% to 59.89% | 1.707 |
| > 230.8 | 84.62 | 54.55% to 98.08% | 51.3 | 41.81% to 60.73% | 1.738 |
| > 231.7 | 84.62 | 54.55% to 98.08% | 52.17 | 42.66% to 61.57% | 1.769 |
| > 232.6 | 84.62 | 54.55% to 98.08% | 53.04 | 43.51% to 62.41% | 1.802 |
| > 233.5 | 84.62 | 54.55% to 98.08% | 53.91 | 44.37% to 63.25% | 1.836 |
| > 238.2 | 84.62 | 54.55% to 98.08% | 54.78 | 45.23% to 64.08% | 1.871 |
| > 243.1 | 84.62 | 54.55% to 98.08% | 55.65 | 46.09% to 64.91% | 1.908 |
| > 244 | 84.62 | 54.55% to 98.08% | 56.52 | 46.96% to 65.74% | 1.946 |
| > 244.7 | 84.62 | 54.55% to 98.08% | 57.39 | 47.83% to 66.56% | 1.986 |
| > 247.5 | 84.62 | 54.55% to 98.08% | 58.26 | 48.7% to 67.39% | 2.027 |
| > 250.2 | 84.62 | 54.55% to 98.08% | 59.13 | 49.57% to 68.21% | 2.07 |
| > 250.5 | 84.62 | 54.55% to 98.08% | 60 | 50.45% to 69.02% | 2.115 |
| > 250.7 | 84.62 | 54.55% to 98.08% | 60.87 | 51.33% to 69.84% | 2.162 |
| > 251.6 | 84.62 | 54.55% to 98.08% | 61.74 | 52.21% to 70.65% | 2.212 |
| > 252.4 | 84.62 | 54.55% to 98.08% | 62.61 | 53.1% to 71.45% | 2.263 |
| > 254.2 | 84.62 | 54.55% to 98.08% | 63.48 | 53.99% to 72.26% | 2.317 |
| > 257.2 | 84.62 | 54.55% to 98.08% | 64.35 | 54.88% to 73.06% | 2.373 |
| > 259 | 84.62 | 54.55% to 98.08% | 65.22 | 55.77% to 73.86% | 2.433 |
| > 260.8 | 84.62 | 54.55% to 98.08% | 66.09 | 56.67% to 74.65% | 2.495 |
| > 263.3 | 84.62 | 54.55% to 98.08% | 66.96 | 57.57% to 75.44% | 2.561 |
| > 267.1 | 84.62 | 54.55% to 98.08% | 67.83 | 58.47% to 76.23% | 2.63 |
| > 270.9 | 84.62 | 54.55% to 98.08% | 68.7 | 59.38% to 77.02% | 2.703 |
| > 272.3 | 84.62 | 54.55% to 98.08% | 69.57 | 60.29% to 77.8% | 2.78 |
| > 272.7 | 84.62 | 54.55% to 98.08% | 70.43 | 61.21% to 78.58% | 2.862 |
| > 273.3 | 84.62 | 54.55% to 98.08% | 71.3 | 62.12% to 79.35% | 2.949 |
| > 277.9 | 84.62 | 54.55% to 98.08% | 72.17 | 63.05% to 80.13% | 3.041 |
| > 282.9 | 84.62 | 54.55% to 98.08% | 73.04 | 63.97% to 80.89% | 3.139 |
| > 283.9 | 84.62 | 54.55% to 98.08% | 73.91 | 64.9% to 81.66% | 3.244 |
| > 286.3 | 84.62 | 54.55% to 98.08% | 74.78 | 65.83% to 82.42% | 3.355 |
| > 289.3 | 84.62 | 54.55% to 98.08% | 75.65 | 66.77% to 83.17% | 3.475 |
| > 290.4 | 84.62 | 54.55% to 98.08% | 76.52 | 67.71% to 83.92% | 3.604 |
| > 294.2 | 84.62 | 54.55% to 98.08% | 77.39 | 68.65% to 84.67% | 3.743 |
| > 298 | 84.62 | 54.55% to 98.08% | 78.26 | 69.6% to 85.41% | 3.892 |
| > 300.4 | 84.62 | 54.55% to 98.08% | 79.13 | 70.56% to 86.15% | 4.054 |
| > 302.7 | 84.62 | 54.55% to 98.08% | 80 | 71.52% to 86.88% | 4.231 |
| > 304 | 84.62 | 54.55% to 98.08% | 80.87 | 72.48% to 87.61% | 4.423 |
| > 310.4 | 84.62 | 54.55% to 98.08% | 81.74 | 73.45% to 88.33% | 4.634 |
| > 318.3 | 84.62 | 54.55% to 98.08% | 82.61 | 74.43% to 89.04% | 4.865 |
| > 321.9 | 84.62 | 54.55% to 98.08% | 83.48 | 75.41% to 89.75% | 5.121 |
| > 324.4 | 84.62 | 54.55% to 98.08% | 84.35 | 76.4% to 90.45% | 5.406 |
| > 326.2 | 84.62 | 54.55% to 98.08% | 85.22 | 77.39% to 91.15% | 5.724 |
| > 328.7 | 84.62 | 54.55% to 98.08% | 86.09 | 78.39% to 91.83% | 6.082 |
| > 331 | 84.62 | 54.55% to 98.08% | 86.96 | 79.4% to 92.51% | 6.487 |
| > 340.6 | 84.62 | 54.55% to 98.08% | 87.83 | 80.42% to 93.18% | 6.951 |
| > 351.1 | 84.62 | 54.55% to 98.08% | 88.7 | 81.45% to 93.84% | 7.485 |
| > 353.1 | 84.62 | 54.55% to 98.08% | 89.57 | 82.48% to 94.49% | 8.109 |
| > 356.7 | 84.62 | 54.55% to 98.08% | 90.43 | 83.53% to 95.13% | 8.846 |
| > 370.3 | 84.62 | 54.55% to 98.08% | 91.3 | 84.59% to 95.75% | 9.731 |
| > 381.9 | 84.62 | 54.55% to 98.08% | 92.17 | 85.66% to 96.36% | 10.81 |
| > 383.7 | 84.62 | 54.55% to 98.08% | 93.04 | 86.75% to 96.95% | 12.16 |
| > 390.2 | 84.62 | 54.55% to 98.08% | 93.91 | 87.86% to 97.52% | 13.9 |
| > 396.9 | 84.62 | 54.55% to 98.08% | 94.78 | 88.99% to 98.06% | 16.22 |
| > 400.4 | 84.62 | 54.55% to 98.08% | 95.65 | 90.15% to 98.57% | 19.46 |
| > 419.6 | 84.62 | 54.55% to 98.08% | 96.52 | 91.33% to 99.04% | 24.33 |
| > 437.2 | 84.62 | 54.55% to 98.08% | 97.39 | 92.57% to 99.46% | 32.44 |
| > 441 | 84.62 | 54.55% to 98.08% | 98.26 | 93.86% to 99.79% | 48.65 |
| > 451.3 | 84.62 | 54.55% to 98.08% | 99.13 | 95.25% to 99.98% | 97.31 |
| > 462.4 | 84.62 | 54.55% to 98.08% | 100 | 96.84% to 100% | |
| > 494.8 | 76.92 | 46.19% to 94.96% | 100 | 96.84% to 100% | |
| > 545.1 | 69.23 | 38.57% to 90.91% | 100 | 96.84% to 100% | |
| > 586.5 | 61.54 | 31.58% to 86.14% | 100 | 96.84% to 100% | |
| > 619.6 | 53.85 | 25.13% to 80.78% | 100 | 96.84% to 100% | |
| > 633.9 | 46.15 | 19.22% to 74.87% | 100 | 96.84% to 100% | |
| > 736.9 | 38.46 | 13.86% to 68.42% | 100 | 96.84% to 100% | |
| > 865.8 | 30.77 | 9.092% to 61.43% | 100 | 96.84% to 100% | |
| > 892.6 | 23.08 | 5.038% to 53.81% | 100 | 96.84% to 100% | |
| > 976.4 | 15.38 | 1.921% to 45.45% | 100 | 96.84% to 100% | |
| > 1065 | 7.692 | 0.1946% to 36.03% | 100 | 96.84% to 100% | |

TABLE 17

Full ROC Data for ASC 6^{th} collection in CSF

| Cutoff (pg/ml) | Sensitivity% | 95% CI | Specificity% | 95% CI | Likelihood ratio |
|---|---|---|---|---|---|
| > 40.63 | 100 | 63.06% to 100% | 7.143 | 0.1807% to 33.87% | 1.077 |
| > 40.67 | 100 | 63.06% to 100% | 14.29 | 1.779% to 42.81% | 1.167 |
| > 41.64 | 100 | 63.06% to 100% | 21.43 | 4.658% to 50.8% | 1.273 |
| > 42.71 | 100 | 63.06% to 100% | 28.57 | 8.389% to 58.1% | 1.4 |
| > 43.09 | 100 | 63.06% to 100% | 35.71 | 12.76% to 64.86% | 1.556 |
| > 43.68 | 100 | 63.06% to 100% | 42.86 | 17.66% to 71.14% | 1.75 |
| > 45.92 | 100 | 63.06% to 100% | 50 | 23.04% to 76.96% | 2 |
| > 48.29 | 100 | 63.06% to 100% | 57.14 | 28.86% to 82.34% | 2.333 |
| > 50.25 | 100 | 63.06% to 100% | 64.29 | 35.14% to 87.24% | 2.8 |
| > 52.18 | 100 | 63.06% to 100% | 71.43 | 41.9% to 91.61% | 3.5 |
| > 53.27 | 100 | 63.06% to 100% | 78.57 | 49.2% to 95.34% | 4.667 |
| > 57.07 | 100 | 63.06% to 100% | 85.71 | 57.19% to 98.22% | 7 |
| > 64.81 | 100 | 63.06% to 100% | 92.86 | 66.13% to 99.82% | 14 |
| > 74.33 | 100 | 63.06% to 100% | 100 | 76.84% to 100% | |
| > 84.74 | 87.5 | 47.35% to 99.68% | 100 | 76.84% to 100% | |
| > 103.3 | 75 | 34.91% to 96.81% | 100 | 76.84% to 100% | |
| > 117.3 | 62.5 | 24.49% to 91.48% | 100 | 76.84% to 100% | |
| > 122.5 | 50 | 15.7% to 84.3% | 100 | 76.84% to 100% | |
| > 268.5 | 37.5 | 8.523% to 75.51% | 100 | 76.84% to 100% | |
| > 504.9 | 25 | 3.185% to 65.09% | 100 | 76.84% to 100% | |
| > 830.8 | 12.5 | 0.316% to 52.65% | 100 | 76.84% to 100% | |

TABLE 18

Full ROC Data for IL-18 1^{st} collection in CSF

| Cutoff (pg/ml) | Sensitivity% | 95% CI | Specificity % | 95% CI | Likelihood ratio |
|---|---|---|---|---|---|
| > 1.167 | 100 | 78.2% to 100% | 5.263 | 0.1332% to 26.03% | 1.056 |
| > 1.298 | 100 | 78.2% to 100% | 10.53 | 1.301% to 33.14% | 1.118 |
| > 1.358 | 100 | 78.2% to 100% | 15.79 | 3.383% to 39.58% | 1.188 |
| > 1.406 | 93.33 | 68.05% to 99.83% | 21.05 | 6.052% to 45.57% | 1.182 |
| > 1.499 | 93.33 | 68.05% to 99.83% | 26.32 | 9.147% to 51.2% | 1.267 |
| > 1.608 | 93.33 | 68.05% to 99.83% | 31.58 | 12.58% to 56.55% | 1.364 |
| > 1.737 | 93.33 | 68.05% to 99.83% | 36.84 | 16.29% to 61.64% | 1.478 |
| > 1.844 | 86.67 | 59.54% to 98.34% | 36.84 | 16.29% to 61.64% | 1.372 |
| > 1.91 | 86.67 | 59.54% to 98.34% | 42.11 | 20.25% to 66.5% | 1.497 |
| > 2.024 | 86.67 | 59.54% to 98.34% | 47.37 | 24.45% to 71.14% | 1.647 |
| > 2.11 | 86.67 | 59.54% to 98.34% | 52.63 | 28.86% to 75.55% | 1.83 |
| > 2.188 | 86.67 | 59.54% to 98.34% | 57.89 | 33.5% to 79.75% | 2.058 |
| > 2.474 | 80 | 51.91% to 95.67% | 57.89 | 33.5% to 79.75% | 1.9 |
| > 2.698 | 80 | 51.91% to 95.67% | 63.16 | 38.36% to 83.71% | 2.171 |
| > 2.722 | 80 | 51.91% to 95.67% | 68.42 | 43.45% to 87.42% | 2.533 |
| > 2.758 | 73.33 | 44.9% to 92.21% | 68.42 | 43.45% to 87.42% | 2.322 |
| > 2.817 | 73.33 | 44.9% to 92.21% | 73.68 | 48.8% to 90.85% | 2.787 |
| > 2.865 | 73.33 | 44.9% to 92.21% | 78.95 | 54.43% to 93.95% | 3.483 |
| > 2.945 | 73.33 | 44.9% to 92.21% | 84.21 | 60.42% to 96.62% | 4.644 |
| > 3.23 | 66.67 | 38.38% to 88.18% | 84.21 | 60.42% to 96.62% | 4.222 |
| > 3.586 | 66.67 | 38.38% to 88.18% | 89.47 | 66.86% to 98.7% | 6.333 |
| > 3.747 | 66.67 | 38.38% to 88.18% | 94.74 | 73.97% to 99.87% | 12.67 |
| > 3.806 | 60 | 32.29% to 83.66% | 94.74 | 73.97% to 99.87% | 11.4 |
| > 3.879 | 60 | 32.29% to 83.66% | 100 | 82.35% to 100% | |
| > 4.254 | 53.33 | 26.59% to 78.73% | 100 | 82.35% to 100% | |
| > 5.826 | 46.67 | 21.27% to 73.41% | 100 | 82.35% to 100% | |
| > 8.428 | 40 | 16.34% to 67.71% | 100 | 82.35% to 100% | |
| > 10.31 | 33.33 | 11.82% to 61.62% | 100 | 82.35% to 100% | |
| > 14.29 | 26.67 | 7.787% to 55.1% | 100 | 82.35% to 100% | |
| > 18.52 | 20 | 4.331% to 48.09% | 100 | 82.35% to 100% | |
| > 21.1 | 13.33 | 1.658% to 40.46% | 100 | 82.35% to 100% | |
| > 24.64 | 6.667 | 0.1686% to 31.95% | 100 | 82.35% to 100% | |

TABLE 19

Full ROC Data for ASC 4^{th} collection in serum (favorable vs. unfavorable)

| Cutoff (pg/ml) | Sensitivity% | 95% CI | Specificity % | 95% CI | Likelihood ratio |
|---|---|---|---|---|---|
| > 194.1 | 100 | 76.84% to 100% | 16.67 | 0.4211% to 64.12% | 1.2 |
| > 240.2 | 100 | 76.84% to 100% | 33.33 | 4.327% to 77.72% | 1.5 |
| > 254.2 | 92.86 | 66.13% to 99.82% | 33.33 | 4.327% to 77.72% | 1.393 |
| > 304.9 | 92.86 | 66.13% to 99.82% | 50 | 11.81% to 88.19% | 1.857 |
| > 374.1 | 85.71 | 57.19% to 98.22% | 50 | 11.81% to 88.19% | 1.714 |
| > 404.7 | 85.71 | 57.19% to 98.22% | 66.67 | 22.28% to 95.67% | 2.571 |
| > 457.6 | 85.71 | 57.19% to 98.22% | 83.33 | 35.88% to 99.58% | 5.143 |
| > 547.6 | 85.71 | 57.19% to | 100 | 54.07% to 100% | |
| | | 98.22% | | | |
| > 605.1 | 78.57 | 49.2% to 95.34% | 100 | 54.07% to 100% | |
| > 623.8 | 71.43 | 41.9% to 91.61% | 100 | 54.07% to 100% | |
| > 636.5 | 64.29 | 35.14% to 87.24% | 100 | 54.07% to 100% | |
| > 647 | 57.14 | 28.86% to 82.34% | 100 | 54.07% to 100% | |
| > 663.7 | 50 | 23.04% to 76.96% | 100 | 54.07% to 100% | |
| > 716.7 | 42.86 | 17.66% to 71.14% | 100 | 54.07% to 100% | |
| > 769 | 35.71 | 12.76% to 64.86% | 100 | 54.07% to 100% | |
| > 828.4 | 28.57 | 8.389% to 58.1% | 100 | 54.07% to 100% | |
| > 944.7 | 21.43 | 4.658% to 50.8% | 100 | 54.07% to 100% | |
| > 1061 | 14.29 | 1.779% to 42.81% | 100 | 54.07% to 100% | |
| > 1118 | 7.143 | 0.1807% to 33.87% | 100 | 54.07% to 100% | |

### Example 4: Examination of Inflammasome Proteins as Biomarkers of Mild Cognitive Impairment (MCI)

### Introduction

A biomarker is a characteristic that can be measured objectively and evaluated as an indicator of normal or pathologic biological processes¹. Important to the care of patients with MCI is the need for biomarkers that can predict onset, exacerbation as well as response to treatment. Additionally, there is a need for a minimally invasive method of harvesting these biomarkers for analysis.

### Methods

### Participants

In this example, samples were purchased from BioIVT. Sample donors were enrolled in the study “Prospective Collection of Samples for Research” sponsored by SeraTrials, LLC. with IRB number 20170439. Here, serum samples from 72 normal male and female donors in the age range of 50 and 68 as well as from 32 male and female patients diagnosed with MCI (Table 20) in the age range of 56 to 91 were analyzed.

TABLE 20

Demographics of participants in MCI study

| Age | Gender | Race | Diagnosis | Medications | Historical Test |
|---|---|---|---|---|---|
| 83 | Male | Caucasian | Mild Cognitive Impairment (MCI), Prostate Cancer, Methicillin Resistant Staphylococcus Aureus Infection, Hyperlipidemia (HLD), Hypertension (HTN), Diverticulitis, Amnesia | Omega 3 1000 mg, Plavix 75 mg, Toprol 50 mg, Vitamin B12-Folic Acid 0.5 mg-1 mg, Vitamin D 400iu, Zetia 10 mg | ARIC MRI Cognitive Function Score= 18 (Feb. 20, 2018) |
| 81 | Female | Caucasian | Mild Cognitive Impairment (MCI), Type 2 Diabetes, Hypercholesterolemia | Aspirin 81 mg, Gabapentin 100 m, Eliquis 2.5 mg, Ranitidine 150 mg, Aricept 10 mg | ARIC MRI Cognitive Function Score= 18 (May 22, 2018) |
| 62 | Male | Caucasian | Mild Cognitive Impairment (MCI), Type 2 Diabetes, Hypertension (HTN), Hyperlipidemia (HLD), Asthma | Omeprazole 20 mg, Benicar 40 mg-12.5 mg, Metformin HCL 500 mg, Glucotrol XL 5 mg, Singulair 10 mg, Clobetasol Propionate 0.05%, Glipizide 5 mg, Advair Diskus 250 mcg-50 mcg, Crestor 10 mg, Ipratropium-Albuterol 0.5 mg-2.5 mg/3 mL, Ventolin HFA 108 mcg | ARIC MRI Cognitive Function Score= 30 (May 15, 2018) |
| 69 | Female | Caucasian | Mild Cognitive Impairment (MCI), Asthma, Chronic Obstructive Pulmonary Disease (COPD), Hypertension (HTN) | Alendronate 70 mg, Meclizine 12.5 mg, Prozac 40 mg, Seroquel 50 mg, Trilipix 54 mg | ARIC MRI Cognitive Function Score= 21 (May 30, 2018) |
| 75 | Male | Caucasian | Mild Cognitive Impairment (MCI), Colon Cancer | Vitamin B12 2500iu, Avastin, Adrucil, Amoxicillin 500 mg, Lisinopril 20 mg, Metformin HCL 500 mg | ARIC MRI Cognitive Function Score= 12 (Mar. 27, 2018) |
| 72 | Male | Caucasian | Mild Cognitive Impairment (MCI), Benign Prostatic Hyperplasia (BPH), Lumbar Spondylosis, Barrett’s Esophagous, Atrial Ectopy, Hypertension (HTN) | Tamsulosin HCL 0.4 mg, Finasteride 5 mg, Multivitamin, Fish Oil 1000 mg, Viagra 100 mg, Tramadol HCL 50 mg | ARIC MRI Cognitive Function Score= 15 (May 10, 2018) |
| 64 | Male | Caucasian | Mild Cognitive Impairment (MCI), Type 2 Diabetes, Hypertension (HTN), Hypercholesterolemia, Benign Prostatic Hyperplasia (BPH) | Zolpidem 10 mg, Cialis 5 mg, Aspirin 81 mg, Tamsulosin 0.4 mg, Rosuvastatin 20 mg, Metformin 500 mg | ARIC MRI Cognitive Function Score= 34 (Apr. 4, 2018) |
| 84 | Female | Caucasian | Mild Cognitive Impairment (MCI), Hypertension (HTN), Hallucinations, Psychoses, Cellulitis, Dementia, Mitral Valve Prolapse (MVP), Hyperlipidemia (HLD), Alzheimer’s Disease (AD) | Simvastatin 20 mg, Potassium Chloride 10 mEq, Amlodipine Besylate 2.5 mg, Dutasteride 0.5 mg, Losartan Potassium 100 mg, Aspirin 81 mg, Furosemide 20 mg, Potassium Chloride 10 mEq, Avodart 0.4 mg, Amlodipine Besylate 2.5 mg, Ramipril 10 mg | ARIC MRI Cognitive Function Score= 8 (May 10, 2018) |
| 68 | Female | Caucasian | Mild Cognitive Impairment (MCI), Multiple Sclerosis | Tysabri, Lexapro, Gabapentin | ARIC MRI Cognitive Function Score= 15 (Apr. 6, 2018) |
| 69 | Female | Caucasian | Mild Cognitive Impairment (MCI), Hypercholesterolemia, Hypertension (HTN), Type 2 Diabetes, Premature Ventricular Contraction | Crestor 5 mg, Omega 3, Zolpidem Tartrate 5 mg, Glucosamine 1500 mg, Fiber, Calcium, Multivitamin, Zyrtec, Chlordiazepoxide -Clidinium 5 mg-2.5 mg, Valacyclovir 500 mg, Lisinopril 10 mg, Janumet 50 mg-500 mg, Metoprolol Succinate 25 mg, Levothyroxine Sodium 100 mcg, Rosuvastatin Calcium 5 mg, Omega 3-Acid Ethyl Esters 1 g, Trazodone 50 mg | ARIC MRI Cognitive Function Score= 33 (May 1, 2018) |
| 50 | Female | Caucasian | Mild Cognitive Impairment (MCI), Hypercholesterolemia | None | ARIC MRI Cognitive Function Score= 30 (Apr. 24, 2018) |
| 78 | Male | Caucasian | Mild Cognitive Impairment (MCI) | Zaleplon 10 mg, Lorazepam 1 mg, Plavix 75 mg, Aspirin, Allopurinol 300 mg, Levothyroxine Sodium 125 mcg, Atorvastatin Calcium 20 mg, Metformin HCL 1000 mg, Pantoprazole Sodium 40 mg | ARIC MRI Cognitive Function Score= 24 (Apr. 27, 2018) |
| 77 | Male | Caucasian | Mild Cognitive Impairment (MCI), Hypertension (HTN), Hyperlipidemia (HLD), Vitamin D Deficiency | Aciphex 20 mg, Citric Acid-D Gluconic Acid, Avodart 0.5 mg, Cozaar 100 mg, Ranitidine Acid Reducer 75 mg, Polyethylene Glycol, Miralax, Symbicort 80 mcg-4.5 mcg, Proair 108 mcg, Ipratropium Bromide 0.03%, Prevacid 15 mg, Losartan Potassium 100 mg, Levocetirizine Dihydrochloride 5 mg, Cialis 5 mg, Albuterol, Rabeprazole Sodium 20 mg, Atorvastatin Calcium 20 mg | ARIC MRI Cognitive Function Score= 24 (May 9, 2018) |
| 73 | Female | Caucasian | Mild Cognitive Impairment (MCI), Hypercholesterolemia, Hypothyroidism, Hypothyroidism, Gastroesophageal Reflux Disease (GERD), Vitamin D Deficiency, Hypertension (HTN) | Rabeprazole Sodium 20 mg, Synthroid 75mcg, Crestor 5 mg, Zyrtec Allergy 10 mg, Aspirin, Calcium 150 mg, CoQ 10 400 mg, Aciphex 20 mg, Zenpep 3000iu-10,000iu, Ipratropium Bromide 0.03%, Rosuvastatin Calcium 5 mg | ARIC MRI Cognitive Function Score= 37 (May 9, 2018) |
| 71 | Male | Caucasian | Mild Cognitive Impairment (MCI), Dyslipidemia, Valvular Heart Disease, Hypertension (HTN), Hyperlipidemia (HLD), Aortic Aneurysm, Ulcerative Colitis (UC) | Epipen, Metoprolol Succinate ER 50 mg, Zyrtec, Montelukast, Pepcid, Tramadol 50 mg, Diazepam 5 mg, Metamucil 48.57%, Aspirin 81 mg, Plavix 75 mg, Nexium 40 mg, Lipitor 10 mg, Asacol 800 mg | ARIC MRI Cognitive Function Score= 24 (May 10, 2018) |
| 74 | Female | Caucasian | Mild Cognitive Impairment (MCI), Asthma, Chronic Obstructive Pulmonary Disease (COPD), Type 2 Diabetes, Hypercholesterolemia, Congestive Heart Failure (CHF), Hypothyroidism | Levothyroxine 75 mg, Metformin 500 mg, Losartan 10 mg, Symbicort, Proventil, Calcium, Vitamin D3, Zyrtec 10 mg | ARIC MRI Cognitive Function Score= 30 (May 11, 2018) |
| 75 | Male | Caucasian | Mild Cognitive Impairment (MCI), Neuropathy, Benign Prostatic Hyperplasia (BPH), Hypertension (HTN), Rheumatoid Arthritis (RA), Sjogren’s Syndrome, Glaucoma, Allergic Rhinitis, Nasal Obstruction, Type 2 Diabetes | Patanase 0.6%, Timolol Hemihydrate, Latanoprost 0.005%, Methotrexate, Prednisone, Folic Acid, Vitamin D, Finasteride 5 mg, Tamsulosin HCL 0.4 mg, Gabapentin 100 mg, Vicodin 5 mg-300 mg, Losartan Potassium 50 mg, Pilocarpine HCL 5 mg, Calcium 600 mg, Vitamin B12 100 mcg, Docusate Sodium 100 mg, Miralax, Polyethylene Glycol, Ventolin HFA 90 mcg, Azithromycin 250 mg, Lasix 20 mg, Levaquin 500 mg, Evoxac 30 mg | ARIC MRI Cognitive Function Score= Refused (May 18, 2018) |
| 75 | Male | Caucasian | Mild Cognitive Impairment (MCI), Hypercholesterolemia, Thyroid Disease | Levothyroxine Sodium 25 mcg, Crestor 40 mg | ARIC MRI Cognitive Function Score= 35 (May 24, 2018) |
| 75 | Male | Caucasian | Mild Cognitive Impairment (MCI), Hypercholesterolemia, Age Related Macular Degeneration (AMD), Erectile Dysfunction (ED) | Pravachol 40 mg, Ocuvite, Viagra 50 mg | ARIC MRI Cognitive Function Score= 31 (Feb. 19, 2018) |
| 75 | Female | Caucasian | Mild Cognitive Impairment (MCI), Type 2 Diabetes, Hypertension (HTN), Dyslipidemia, Chronic Kidney Disease (CKD), Pulmonary Nodule, Hyperlipidemia (HLD) | Metformin 500 mg, Atorvastatin Calcium 20 mg, Cozaar 100 mg, Aspirin 81 mg, Hydrochlorothiaz ide 25 mg, Lipitor 20 mg | ARIC MRI Cognitive Function Score= 42 (May 1, 2018) |
| 76 | Female | Caucasian | Mild Cognitive Impairment (MCI), Hyperlipidemia (HLD), Hypertension (HTN), Gastroesophageal Reflux Disease (GERD), Anxiety, Alzheimer’s Disease (AD), Hypothyroidism | Donepezil HCL 10 mg, Levothyroxine Sodium 50 mcg, Tramadol HCL 50 mg, Atorvastatin Calcium 20 mg, Omeprazole 20 mg, Losartan Potassium 50 mg, Aricept 10 mg, Paxil 20 mg, Namenda 10 mg | ARIC MRI Cognitive Function Score= 7 (May 4, 2018) |
| 76 | Male | Caucasian | Mild Cognitive Impairment (MCI), Hypertension (HTN), Type 2 Diabetes, Peripheral Polyneuropathy, Benign Prostatic Hyperplasia (BPH) | Novolog, Lantus 100iu/mL, Metoprolol Succinate 25 mg, Tacrolimus, Terazosin HCL 10 mg, CellCept 250 mg, Aspirin 81 mg, Allopurinol 150 mg, Atorvastatin Calcium 10 mg, Losartan Potassium 100 m | ARIC MRI Cognitive Function Score= 28 (May 15, 2018) |
| 67 | Female | Caucasian | Mild Cognitive Impairment (MCI), Asthma, Hypercholesterolemia | Crestor 40 mg, Omeprazole 20 mg | ARIC MRI Cognitive Function Score= 40 (May 7, 2018) |
| 56 | Female | Caucasian /Japanese | Mild Cognitive Impairment (MCI) | Daily Vitamins, Aspirin 81 mg | ARIC MRI Cognitive Function Score= 41 (May 8, 2018) |
| 58 | Female | Caucasian | Mild Cognitive Impairment (MCI), Hyperlipidemia (HLD) | Simvastatin 20 mg, Caltrate 600 mg-Vitamin D 800iu, Vitamin D 2000iu, Ibuprofen 800 mg, Prolia 60 mg/mL | ARIC MRI Cognitive Function Score= 42 (May 8, 2018) |
| 75 | Female | Caucasian | Mild Cognitive Impairment (MCI), AF, Dyslipidemia, Hypertension (HTN), Hypothyroidism | Crestor 10 mg, Armour Thyroid 60 mg, Ramipril 5 mg, Hydrochlorothiaz ide 25 mg, Promethium 200 mg, Augmentin 875 mg-125 mg, Rosuvastatin Calcium 10 mg | ARIC MRI Cognitive Function Score= 31 (May 11, 2018) |
| 84 | Female | Caucasian | Mild Cognitive Impairment (MCI), Venous Insufficiency, Hyperlipidemia (HLD), Hypothyroidism, Parkinson’s Disease (PD), Mitral Valve Prolapse (MVP), Anxiety | Cipro 500 mg, Ibuprofen 800 mg, Xanax 0.5 mg, Fluconazole 150 mg, Carbidopa-Levodopa 25 mg-100 mg, Potassium Chloride 20 mEq, Simvastatin 20 mg, Furosemide 40 mg, Levothyroxine Sodium 75 mcg, Atenolol 25 mg, Lasix, Aspirin 81 mg, Acetaminophen 500 mg | ARIC MRI Cognitive Function Score= 19 (May 11, 2018) |
| 88 | N/A | Caucasian | Mild Cognitive Impairment (MCI), Hyperlipidemia (HLD), Peripheral Vascular Disease, Hypertension (HTN), Hyperlipidemia, Mild Intermittent Asthma, Hypercholesterolemia, Type 2 Diabetes, Alzheimer’s Disease (AD) | Cozaar 100 mg, Crestor 10 mg, Aspirin, Prilosec 20 mg, Amlodipine Besylate 5 mg, D3 1000iu, Vitamin C 100 mg, Multi for Him, Omeprazole 20 mg | ARIC MRI Cognitive Function Score= 8 (May 22, 2018) |
| 71 | Male | Caucasian | Mild Cognitive Impairment (MCI), Hypertension (HTN), Hypercholesterolemia, Chronic Kidney Disease (CKD), Palsy of Conjugate Gaze, Short Term Memory, Hyperlipidemia, Cervical Spondylosis, Basal Cell Cancer (BCC), Complex Partial Epileptic Seizure, Chronic Tremor, Lumbosacral Radiculitis, Allergic Rhinitis, Lumbar Arthritis, Arthritis, Bilateral Hearing Loss | Aspirin 81 mg, Brimonidine 0.15%, Cialis 20 mg, Dexamethasone 4 mg/ml, Donepezil 5 mg, Fexofenadine 180 mg, Lamotrigine 200 mg, Lisinopril 5 mg, Meloxicam 15 mg, Pramipexole 0.25 mg, Simvastatin 40 mg, Virtussin 10 mg-100 mg/5 ml | ARIC MRI Cognitive Function Score= 44 (May 24, 2018) |
| 86 | Male | Caucasian | Mild Cognitive Impairment (MCI), Hypertensive Heart and Renal Disease with Congestive Heart Failure, Cyst and Pseudocyst of Pancreas, Benign Prostatic Hyperplasia (BPH), Type 2 Diabetes, Chronic Kidney Disease (CKD), Hypokalemia, Chronic Systolic Heart Disease, Mitral Valve Prolapse (MVP), Atrial Fibrillation (AF), Hyperlipidemia, Sensorineural Hearing Loss, Left Bundle Branch Block, Pulmonary Hypertension (HTN), Hyperparathyroidism | Amlodipine 5 mg, Glimepiride 1 mg, Nitroglycerin 0.2 mg, Potassium Chloride 20 meq, Warfarin 2 mg | ARIC MRI Cognitive Function Score= 48 (May 17, 2018) |
| 91 | Female | Caucasian | Mild Cognitive Impairment (MCI), Type 2 Diabetes, Hypertension (HTN), Hypercholesterolemia, Benign Prostate Hyperplasia (BPH), Abdominal Aortic Aneursym, Atrial Fibrillation (AF) | Amlodipine Besylate 5 mg, Atorvastatin Calcium 40 mg, Coumadin, Plavix 75 mg, Toprol 50 mg | ARIC MRI Cognitive Function Score= 31 (Mar. 13, 2018) |
| 88 | Male | Caucasian | Mild Cognitive Impairment (MCI), Hypercholesterolemia, Melanoma, Depression, Squamous Cell Carcinoma, GERD, Hemorrhoids, TIA | Trintellix 10 mg, Aripiprazole 2.5 mg, Rosuvastatin 20 mg, Modafinil 200 mg, Amphetamine 20 mg, Namenda 28 mg, Esomeprazole 20 mg, Lutein 5 mg, Vitamin D3 1000iu, Aspirin 81 mg, Vitamin B12 | ARIC MRI Cognitive Function Score= 16 (Feb. 21, 2018) |

### Simple Plex Assay

Analysis of inflammasome protein (caspase-1, ASC, IL-1β and IL-18) concentration in serum samples from MCI and age-matched controls were performed using the Ella System (Protein System) as described in ^{2, 3}.

### Biomarker Analyses

Data obtained by the Simple Plex assay were analyzed with Prism 7 software (GraphPad). First, outliers were removed and receiver operating characteristics (ROC) were calculated, thus obtaining a 95% confidence interval, a standard deviation and a p-value. P-value of significance was considered at less than 0.05. A cut-off point was then obtained for a range of different specificities and sensitivities and their respective likelihood ratio^{2,}³.

### Results

### ASC and IL-18 are Elevated in the Serum of Patients With MCI

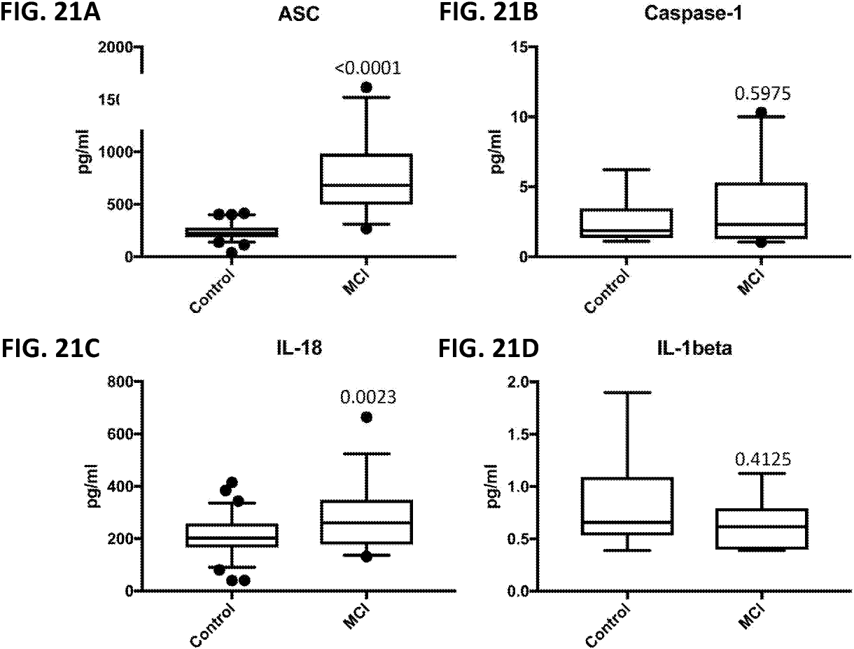

Serum samples from patients with MCI patients and aged-matched healthy donors were analyzed for the protein expression levels of ASC (FIG. 21A), caspase-1 (FIG. 21B), IL-18 (FIG. 21C) and IL-1β (FIG. 21D). Here, the protein levels of ASC and IL-18 were found to be significantly higher in the MCI group when compared to the control group; thus suggesting an involvement of ASC and IL-18 in the pathology of MCI.

### ASC is a Promising Serum Biomarker of MCI

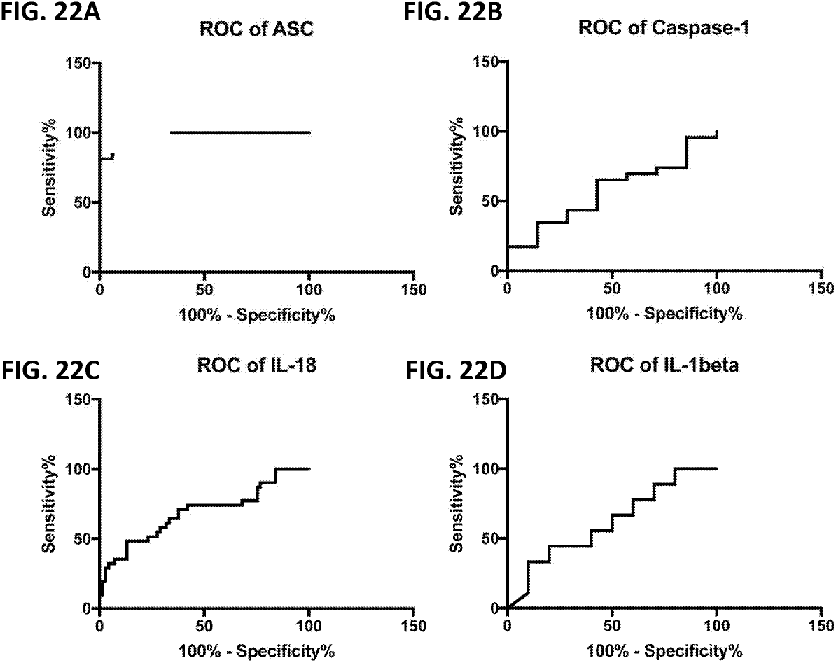

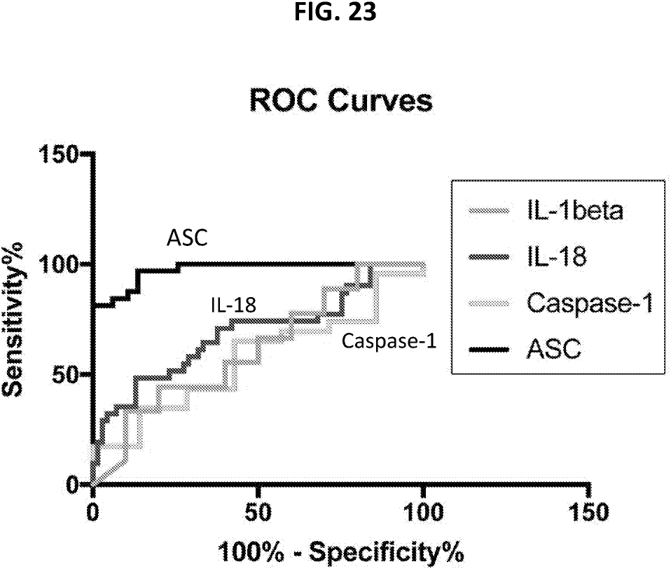

To determine if inflammasome signaling proteins can be used as biomarkers of MCI, the area under the curve (AUC) was determined for caspase-1 (FIG. 22A), ASC (FIG. 22B), IL-1β (FIG. 22C) and IL-18 (FIG. 22D). FIG. 23 shows all of the ROC curves from FIGS. 22A-22D superimposed onto each other. Of all the proteins analyzed, ASC presented the highest AUC of 0.974 (p <0.0001), followed by IL-18 with an AUC of 0.6896 (p = 0.0025) (Table 21). The cut-off point for ASC was 264.9 pg/ml with 100% sensitivity and 74% specificity (see Tables 22 and 23); whereas IL-18 had a cut-off point of 213.9 pg/ml with 74% sensitivity and 58% specificity (Tables 22 and 25). In addition to Table 22, the cut-off points and sensitivity/specificity data for caspase-1 and IL-1beta can be found in Tables 24 and 26, respectively.

TABLE 21

ROC analysis results for inflammasome signaling proteins in serum

| BIOMARKER | AREA | STD. ERROR | 95% C.I. | P VALUE |
|---|---|---|---|---|
| ASC | 0.974 | 0.01301 | 0.9485 to 0.9995 | <0.0001 |
| Caspase-1 | 0.5714 | 0.1174 | 0.3413 to 0.8016 | 0.5728 |
| IL-18 | 0.6896 | 0.06086 | 0.5703 to 0.8089 | 0.0025 |
| IL-1beta | 0.6167 | 0.1317 | 0.3585 to 0.8749 | 0.3913 |

TABLE 22

Cut-off point analyses for inflammasome signaling proteins in serum

| Biomarker | Cut-off point (pg/ml) | Sensitivity (%) | Specificity (%) | PPV (%) | NPV (%) | Likelihood Ratio | Accuracy (%) |
|---|---|---|---|---|---|---|---|
| ASC | >264.9 | 100 | 74 | 65 | 100 | 3.882 | 83 |
| Caspase-1 | >1.753 | 65 | 43 | 79 | 27 | 1.141 | 60 |
| IL-18 | >213.9 | 74 | 58 | 44 | 83 | 1.765 | 63 |
| IL-1beta | <0.684 | 67 | 50 | 55 | 63 | 1.333 | 58 |

TABLE 23

Cut-off point analyses for ASC in serum

| Cutoff | Sensitivity% | 95% CI | Specificity% | 95% CI | Likelihood ratio |
|---|---|---|---|---|---|
| > 76.58 | 100 | 89.11% to 100% | 1.515 | 0.03835% to 8.155% I | 1.015 |
| > 127.1 | 100 | 89.11% to 100% | 3.03 | 0.3691% to 10.52% | 1.031 |
| > 141.6 | 100 | 89.11% to 100% | 4.545 | 0.9474% to 12.71% | 1.048 |
| > 145.8 | 100 | 89.11% to 100% | 6.061 | 1.676% to 14.8% | 1.065 |
| > 148.9 | 100 | 89.11% to 100% | 7.576 | 2.506% to 16.8% | 1.082 |
| > 152.9 | 100 | 89.11% to 100% | 9.091 | 3.41% to 18.74% | 1.1 |
| > 158.1 | 100 | 89.11% to 100% | 10.61 | 4.372% to 20.64% | 1.119 |
| > 159.9 | 100 | 89.11% to 100% | 12.12 | 5.381% to 22.49% | 1.138 |
| > 164.5 | 100 | 89.11% to 100% | 13.64 | 6.43% to 24.31% | 1.158 |
| > 169.3 | 100 | 89.11% to 100% | 15.15 | 7.512% to 26.1% | 1.179 |
| > 171.1 | 100 | 89.11% to 100% | 16.67 | 8.625% to 27.87% | 1.2 |
| > 173.4 | 100 | 89.11% to 100% | 18.18 | 9.764% to 29.61% | 1.222 |
| > 177.1 | 100 | 89.11% to 100% | 19.7 | 10.93% to 31.32% | 1.245 |
| > 180.6 | 100 | 89.11% to 100% | 21.21 | 12.11% to 33.02% | 1.269 |
| > 182 | 100 | 89.11% to 100% | 22.73 | 13.31% to 34.7% | 1.294 |
| > 183.5 | 100 | 89.11% to 100% | 24.24 | 14.54% to 36.36% | 1.32 |
| > 185.5 | 100 | 89.11% to 100% | 25.76 | 15.78% to 38.01% | 1.347 |
| > 188.8 | 100 | 89.11% to 100% | 27.27 | 17.03% to 39.64% | 1.375 |
| > 191.5 | 100 | 89.11% to 100% | 28.79 | 18.3% to 41.25% | 1.404 |
| > 193.1 | 100 | 89.11% to 100% | 30.3 | 19.59% to 42.85% | 1.435 |
| > 194.6 | 100 | 89.11% to 100% | 31.82 | 20.89% to 44.44% | 1.467 |
| > 196.4 | 100 | 89.11% to 100% | 33.33 | 22.2% to 46.01% | 1.5 |
| > 197.6 | 100 | 89.11% to 100% | 34.85 | 23.53% to 47.58% | 1.535 |
| > 198.1 | 100 | 89.11% to 100% | 36.36 | 24.87% to 49.13% | 1.571 |
| > 199.7 | 100 | 89.11% to 100% | 37.88 | 26.22% to 50.66% | 1.61 |
| > 201 | 100 | 89.11% to 100% | 39.39 | 27.58% to 52.19% | 1.65 |
| > 203.1 | 100 | 89.11% to 100% | 40.91 | 28.95% to 53.71% | 1.692 |
| > 210.3 | 100 | 89.11% to 100% | 42.42 | 30.34% to 55.21% | 1.737 |
| > 216.1 | 100 | 89.11% to 100% | 43.94 | 31.74% to 56.7% | 1.784 |
| > 217.9 | 100 | 89.11% to 100% | 45.45 | 33.14% to 58.19% | 1.833 |
| > 219.1 | 100 | 89.11% to 100% | 46.97 | 34.56% to 59.66% | 1.886 |
| > 220.4 | 100 | 89.11% to 100% | 48.48 | 35.99% to 61.12% | 1.941 |
| > 223.3 | 100 | 89.11% to 100% | 50 | 37.43% to 62.57% | 2 |
| > 226.3 | 100 | 89.11% to 100% | 51.52 | 38.88% to 64.01% | 2.063 |
| > 229.5 | 100 | 89.11% to 100% | 53.03 | 40.34% to 65.44% | 2.129 |
| > 232.3 | 100 | 89.11% to 100% | 54.55 | 41.81% to 66.86% | 2.2 |
| > 233.4 | 100 | 89.11% to 100% | 56.06 | 43.3% to 68.26% | 2.276 |
| > 237.3 | 100 | 89.11% to 100% | 57.58 | 44.79% to 69.66% | 2.357 |
| > 241.8 | 100 | 89.11% to 100% | 59.09 | 46.29% to 71.05% | 2.444 |
| > 243.9 | 100 | 89.11% to 100% | 60.61 | 47.81% to 72.42% | 2.538 |
| > 247.1 | 100 | 89.11% to 100% | 62.12 | 49.34% to 73.78% | 2.64 |
| > 250 | 100 | 89.11% to 100% | 63.64 | 50.87% to 75.13% | 2.75 |
| > 251.6 | 100 | 89.11% to 100% | 65.15 | 52.42% to 76.47% | 2.87 |
| > 252.7 | 100 | 89.11% to 100% | 66.67 | 53.99% to 77.8% | 3 |
| > 254.5 | 100 | 89.11% to 100% | 68.18 | 55.56% to 79.11% | 3.143 |
| > 257.2 | 100 | 89.11% to 100% | 69.7 | 57.15% to 80.41% | 3.3 |
| > 259 | 100 | 89.11% to 100% | 71.21 | 58.75% to 81.7% | 3.474 |
| > 260.8 | 100 | 89.11% to 100% | 72.73 | 60.36% to 82.97% | 3.667 |
| > 264.9 | 100 | 89.11% to 100% | 74.24 | 61.99% to 84.22% | 3.882 |
| > 272.4 | 96.88 | 83.78% to 99.92% | 74.24 | 61.99% to 84.22% | 3.761 |
| > 280.5 | 96.88 | 83.78% to 99.92% | 75.76 | 63.64% to 85.46% | 3.996 |
| > 287.5 | 96.88 | 83.78% to 99.92% | 77.27 | 65.3% to 86.69% | 4.263 |
| > 293.1 | 96.88 | 83.78% to 99.92% | 78.79 | 66.98% to 87.89% | 4.567 |
| > 298.4 | 96.88 | 83.78% to 99.92% | 80.3 | 68.68% to 89.07% | 4.918 |
| > 308.7 | 96.88 | 83.78% to 99.92% | 81.82 | 70.39% to 90.24% | 5.328 |
| > 320.8 | 96.88 | 83.78% to 99.92% | 83.33 | 72.13% to 91.38% | 5.813 |
| > 326.2 | 96.88 | 83.78% to 99.92% | 84.85 | 73.9% to 92.49% | 6.394 |
| > 330.5 | 96.88 | 83.78% to 99.92% | 86.36 | 75.69% to 93.57% | 7.104 |
| > 337.7 | 93.75 | 79.19% to 99.23% | 86.36 | 75.69% to 93.57% | 6.875 |
| > 341.9 | 90.63 | 74.98% to 98.02% | 86.36 | 75.69% to 93.57% | 6.646 |
| > 348.5 | 87.5 | 71.01% to 96.49% | 86.36 | 75.69% to 93.57% | 6.417 |
| > 356.6 | 87.5 | 71.01% to 96.49% | 87.88 | 77.51% to 94.62% | 7.219 |
| > 367.5 | 87.5 | 71.01% to 96.49% | 89.39 | 79.36% to 95.63% | 8.25 |
| > 378.6 | 84.38 | 67.21% to 94.72% | 89.39 | 79.36% to 95.63% | 7.955 |
| > 381.9 | 84.38 | 67.21% to 94.72% | 90.91 | 81.26% to 96.59% | 9.281 |
| > 383.6 | 84.38 | 67.21% to 94.72% | 92.42 | 83.2% to 97.49% | 11.14 |
| > 386.8 | 84.38 | 67.21% to 94.72% | 93.94 | 85.2% to 98.32% | 13.92 |
| > 390.2 | 81.25 | 63.56% to 92.79% | 93.94 | 85.2% to 98.32% | 13.41 |
| > 397.1 | 81.25 | 63.56% to 92.79% | 95.45 | 87.29% to 99.05% | 17.88 |
| > 403.4 | 81.25 | 63.56% to 92.79% | 96.97 | 89.48% to 99.63% | 26.81 |
| >409.2 | 81.25 | 63.56% to 92.79% | 98.48 | 91.84% to 99.96% | 53.63 |
| > 414.2 | 81.25 | 63.56% to 92.79% | 100 | 94.56% to 100% | |
| > 455.9 | 78.13 | 60.03% to 90.72% | 100 | 94.56% to 100% | |
| > 498.6 | 75 | 56.6% to 88.54% | 100 | 94.56% to 100% | |
| > 507.3 | 71.88 | 53.25% to 86.25% | 100 | 94.56% to 100% | |
| > 520.5 | 68.75 | 49.99% to 83.88% | 100 | 94.56% to 100% | |
| > 530.4 | 65.63 | 46.81% to 81.43% | 100 | 94.56% to 100% | |
| > 551.7 | 62.5 | 43.69% to 78.9% | 100 | 94.56% to 100% | |
| > 603.5 | 59.38 | 40.64% to 76.3% | 100 | 94.56% to 100% | |
| > 646 | 56.25 | 37.66% to 73.64% | 100 | 94.56% to 100% | |
| > 664.7 | 53.13 | 34.74% to 70.91% | 100 | 94.56% to 100% | |
| > 681.2 | 50 | 31.89% to 68.11% | 100 | 94.56% to 100% | |
| > 691 | 46.88 | 29.09% to 65.26% | 100 | 94.56% to 100% | |
| > 698.5 | 43.75 | 26.36% to 62.34% | 100 | 94.56% to 100% | |
| > 708.9 | 40.63 | 23.7% to 59.36% | 100 | 94.56% to 100% | |
| > 723.1 | 37.5 | 21.1% to 56.31% | 100 | 94.56% to 100% | |
| > 763.6 | 34.38 | 18.57% to 53.19% | 100 | 94.56% to 100% | |
| > 809 | 31.25 | 16.12% to 50.01% | 100 | 94.56% to 100% | |
| > 860.9 | 28.13 | 13.75% to 46.75% | 100 | 94.56% to 100% | |
| > 956.1 | 25 | 11.46% to 43.4% | 100 | 94.56% to 100% | |
| > 1012 | 21.88 | 9.277% to 39.97% | 100 | 94.56% to 100% | |
| > 1109 | 18.75 | 7.208% to 36.44% | 100 | 94.56% to 100% | |
| > 1253 | 15.63 | 5.275% to 32.79% | 100 | 94.56% to 100% | |
| > 1307 | 12.5 | 3.513% to 28.99% | 100 | 94.56% to 100% | |
| > 1333 | 9.375 | 1.977% to 25.02% | 100 | 94.56% to 100% | |
| > 1410 | 6.25 | 0.7661% to 20.81% | 100 | 94.56% to 100% | |
| > 1541 | 3.125 | 0.07909% to 16.22% | 100 | 94.56% to 100% | |

TABLE 24

Cut-off point analyses for caspase-1 in serum

| Cutoff | Sensitivity% | 95% CI | Specificity% | 95% CI | Likelihood ratio |
|---|---|---|---|---|---|
| > 1.076 | 95.65 | 78.05% to 99.89% | 0 | 0% to 40.96% | 0.9565 |
| > 1.136 | 95.65 | 78.05% to 99.89% | 14.29 | 0.361% to 57.87% | 1.116 |
| > 1.171 | 91.3 | 71.96% to 98.93% | 14.29 | 0.361% to 57.87% | 1.065 |
| > 1.177 | 86.96 | 66.41% to 97.22% | 14.29 | 0.361% to 57.87% | 1.014 |
| > 1.197 | 82.61 | 61.22% to 95.05% | 14.29 | 0.361% to 57.87% | 0.9638 |
| > 1.243 | 78.26 | 56.3% to 92.54% | 14.29 | 0.361% to 57.87% | 0.913 |
| > 1.317 | 73.91 | 51.59% to 89.77% | 14.29 | 0.361% to 57.87% | 0.8623 |
| > 1.387 | 73.91 | 51.59% to 89.77% | 28.57 | 3.669% to 70.96% | 1.035 |
| > 1.468 | 69.57 | 47.08% to 86.79% | 28.57 | 3.669% to 70.96% | 0.9739 |
| > 1.58 | 69.57 | 47.08% to 86.79% | 42.86 | 9.899% to 81.59% | 1.217 |
| > 1.753 | 65.22 | 42.73% to 83.62% | 42.86 | 9.899% to 81.59% | 1.141 |
| > 1.882 | 65.22 | 42.73% to 83.62% | 57.14 | 18.41% to 90.1% | 1.522 |
| > 1.941 | 60.87 | 38.54% to 80.29% | 57.14 | 18.41% to 90.1% | 1.42 |
| > 2.093 | 56.52 | 34.49% to 76.81% | 57.14 | 18.41% to 90.1% | 1.319 |
| > 2.251 | 52.17 | 30.59% to 73.18% | 57.14 | 18.41% to 90.1% | 1.217 |
| > 2.391 | 47.83 | 26.82% to 69.41% | 57.14 | 18.41% to 90.1% | 1.116 |
| > 2.592 | 43.48 | 23.19% to 65.51% | 57.14 | 18.41% to 90.1% | 1.014 |
| > 2.736 | 43.48 | 23.19% to 65.51% | 71.43 | 29.04% to 96.33% | 1.522 |
| > 2.915 | 39.13 | 19.71% to 61.46% | 71.43 | 29.04% to 96.33% | 1.37 |
| > 3.263 | 34.78 | 16.38% to 57.27% | 71.43 | 29.04% to 96.33% | 1.217 |
| > 4.06 | 34.78 | 16.38% to 57.27% | 85.71 | 42.13% to 99.64% | 2.435 |
| > 4.774 | 30.43 | 13.21% to 52.92% | 85.71 | 42.13% to 99.64% | 2.13 |
| > 5.103 | 26.09 | 10.23% to 48.41% | 85.71 | 42.13% to 99.64% | 1.826 |
| > 5.44 | 21.74 | 7.46% to 43.7% | 85.71 | 42.13% to 99.64% | 1.522 |
| > 5.896 | 17.39 | 4.951% to 38.78% | 85.71 | 42.13% to 99.64% | 1.217 |
| > 6.366 | 17.39 | 4.951% to 38.78% | 100 | 59.04% to 100% | |
| > 6.624 | 13.04 | 2.775% to 33.59% | 100 | 59.04% to 100% | |
| > 7.76 | 8.696 | 1.071% to 28.04% | 100 | 59.04% to 100% | |
| > 9.548 | 4.348 | 0.11% to 21.95% | 100 | 59.04% to 100% | |

TABLE 25

Cut-off point analyses for IL-18 in serum

| | | | | | Likelihood ratio |
|---|---|---|---|---|---|
| Cutoff | Sensitivity% | 95% CI | Specificity% | 95% CI | |
| | | 88.78% to 100% | | 0.03669% to 7.812% | |
| > 40.42 | 100 | | 1.449 | | 1.015 |
| | | 88.78% to 100% | | 0.353% to 10.08% | |
| > 60.89 | 100 | | 2.899 | | 1.03 |
| | | 88.78% to 100% | | 0.9058% to 12.18% | |
| > 91.28 | 100 | | 4.348 | | 1.045 |
| | | 88.78% to 100% | | 1.602% to 14.18% | |
| > 104.2 | 100 | | 5.797 | | 1.062 |
| | | 88.78% to 100% | | 2.395% to 16.11% | |
| > 109.7 | 100 | | 7.246 | | 1.078 |
| | | 88.78% to 100% | | 3.258% to 17.97% | |
| > 114.8 | 100 | | 8.696 | | 1.095 |
| | | 88.78% to 100% | | 4.177% to 19.79% | |
| > 118.1 | 100 | | 10.14 | | 1.113 |
| | | 88.78% to 100% | | 5.141% to 21.57% | |
| > 121.1 | 100 | | 11.59 | | 1.131 |
| | | 88.78% to 100% | | 6.142% to 23.32% | |
| > 124.2 | 100 | | 13.04 | | 1.15 |
| | | 88.78% to 100% | | 7.175% to 25.04% | |
| > 126.5 | 100 | | 14.49 | | 1.169 |
| | | 88.78% to 100% | | 8.236% to 26.74% | |
| > 129.7 | 100 | | 15.94 | | 1.19 |
| | | 83.3% to 99.92% | | 8.236% to 26.74% | |
| > 136.2 | 96.77 | | 15.94 | | 1.151 |
| | | 78.58% to 99.21% | | 8.236% to 26.74% | |
| > 141.2 | 93.55 | | 15.94 | | 1.113 |
| | | 74.25% to 97.96% | | 8.236% to 26.74% | |
| > 147.3 | 90.32 | | 15.94 | | 1.075 |
| | | 74.25% to 97.96% | | 9.322% to 28.41% | |
| > 152.8 | 90.32 | | 17.39 | | 1.093 |
| | | 74.25% to 97.96% | | 10.43% to 30.06% | |
| > 154 | 90.32 | | 18.84 | | 1.113 |
| | | 74.25% to 97.96% | | 11.56% to 31.69% | |
| > 155.4 | 90.32 | | 20.29 | | 1.133 |
| | | 74.25% to 97.96% | | 12.71% to 33.31% | |
| > 156 | 90.32 | | 21.74 | | 1.154 |
| | | 74.25% to 97.96% | | 13.87% to 34.91% | |
| > 157.8 | 90.32 | | 23.19 | | 1.176 |
| | | 70.17% to 96.37% | | 13.87% to 34.91% | |
| > 161.1 | 87.1 | | 23.19 | | 1.134 |
| | | 70.17% to 96.37% | | 15.05% to 36.49% | |
| > 163.5 | 87.1 | | 24.64 | | 1.156 |
| | | 66.27% to 94.55% | | 15.05% to 36.49% | |
| > 164.8 | 83.87 | | 24.64 | | 1.113 |
| | | 62.53% to 92.55% | | 15.05% to 36.49% | |
| > 166.8 | 80.65 | | 24.64 | | 1.07 |
| | | 58.9% to 90.41% | | 15.05% to 36.49% | |
| > 169.1 | 77.42 | | 24.64 | | 1.027 |
| | | 58.9% to 90.41% | | 16.25% to 38.06% | |
| > 170.8 | 77.42 | | 26.09 | | 1.047 |
| | | 58.9% to 90.41% | | 17.46% to 39.62% | |
| > 171.8 | 77.42 | | 27.54 | | 1.068 |
| | | 58.9% to 90.41% | | 18.69% to 41.16% | |
| > 172.8 | 77.42 | | 28.99 | | 1.09 |
| | | 58.9% to 90.41% | | 19.92% to 42.69% | |
| > 175.2 | 77.42 | | 30.43 | | 1.113 |
| | | 58.9% to 90.41% | | 21.17% to 44.21% | |
| > 177.3 | 77.42 | | 31.88 | | 1.137 |
| | | 55.39% to 88.14% | | 21.17% to 44.21% | |
| > 178.3 | 74.19 | | 31.88 | | 1.089 |
| | | 55.39% to 88.14% | | 22.44% to 45.71% | |
| > 178.9 | 74.19 | | 33.33 | | 1.113 |
| | | 55.39% to 88.14% | | 23.71% to 47.21% | |
| > 179.8 | 74.19 | | 34.78 | | 1.138 |
| | | 55.39% to 88.14% | | 24.99% to 48.69% | |
| > 182 | 74.19 | | 36.23 | | 1.163 |
| | | 55.39% to 88.14% | | 26.29% to 50.17% | |
| > 188.3 | 74.19 | | 37.68 | | 1.191 |
| | | 55.39% to 88.14% | | | |
| > 194.4 | 74.19 | | 39.13 | 27.6% to 51.63% | 1.219 |
| | | 55.39% to 88.14% | | 28.91% to 53.08% | |
| > 196 | 74.19 | | 40.58 | | 1.249 |
| | | 55.39% to 88.14% | | 30.24% to 54.52% | |
| > 197.4 | 74.19 | | 42.03 | | 1.28 |
| | | 55.39% to 88.14% | | 31.58% to 55.96% | |
| > 198.4 | 74.19 | | 43.48 | | 1.313 |
| | | 55.39% to 88.14% | | 32.92% to 57.38% | |
| > 199.3 | 74.19 | | 44.93 | | 1.347 |
| | | 55.39% to 88.14% | | | |
| > 200.6 | 74.19 | | 46.38 | 34.28% to 58.8% | 1.384 |
| | | 55.39% to 88.14% | | | |
| > 201.3 | 74.19 | | 47.83 | 35.65% to 60.2% | 1.422 |
| | | 55.39% to 88.14% | | 37.02% to 61.59% | |
| > 201.9 | 74.19 | | 49.28 | | 1.463 |
| | | 55.39% to 88.14% | | 38.41% to 62.98% | |
| > 202.6 | 74.19 | | 50.72 | | 1.506 |
| | | 55.39% to 88.14% | | | |
| > 206 | 74.19 | | 52.17 | 39.8% to 64.35% | 1.551 |
| | | 55.39% to 88.14% | | | |
| > 210.9 | 74.19 | | 53.62 | 41.2% to 65.72% | 1.6 |
| | | 55.39% to 88.14% | | 42.62% to 67.08% | |
| > 212.9 | 74.19 | | 55.07 | | 1.651 |
| | | 55.39% to 88.14% | | 44.04% to 68.42% | |
| > 213.4 | 74.19 | | 56.52 | | 1.706 |
| | | 55.39% to 88.14% | | 45.48% to 69.76% | |
| > 213.9 | 74.19 | | 57.97 | | 1.765 |
| | | 51.96% to 85.78% | | 45.48% to 69.76% | |
| > 215.4 | 70.97 | | 57.97 | | 1.689 |
| | | 51.96% to 85.78% | | 46.92% to 71.09% | |
| > 217.2 | 70.97 | | 59.42 | | 1.749 |
| | | 51.96% to 85.78% | | | |
| > 219 | 70.97 | | 60.87 | 48.37% to 72.4% | 1.814 |
| | | 51.96% to 85.78% | | 49.83% to 73.71% | |
| > 222.8 | 70.97 | | 62.32 | | 1.883 |
| | | 48.63% to 83.32% | | 49.83% to 73.71% | |
| > 226.4 | 67.74 | | 62.32 | | 1.798 |
| | | 45.37% to 80.77% | | 49.83% to 73.71% | |
| > 227.6 | 64.52 | | 62.32 | | 1.712 |
| | | 45.37% to 80.77% | | 51.31% to 75.01% | |
| > 228 | 64.52 | | 63.77 | | 1.781 |
| | | 45.37% to 80.77% | | 52.79% to 76.29% | |
| > 231.4 | 64.52 | | 65.22 | | 1.855 |
| | | 45.37% to 80.77% | | 54.29% to 77.56% | |
| > 236 | 64.52 | | 66.67 | | 1.935 |
| | | 42.19% to 78.15% | | 54.29% to 77.56% | |
| > 239.1 | 61.29 | | 66.67 | | 1.839 |
| | | 42.19% to 78.15% | | 55.79% to 78.83% | |
| > 241.3 | 61.29 | | 68.12 | | 1.922 |
| | | 39.08% to 75.45% | | 55.79% to 78.83% | |
| > 241.9 | 58.06 | | 68.12 | | 1.821 |
| | | 39.08% to 75.45% | | 57.31% to 80.08% | |
| > 242.1 | 58.06 | | 69.57 | | 1.908 |
| | | 39.08% to 75.45% | | 58.84% to 81.31% | |
| > 243.9 | 58.06 | | 71.01 | | 2.003 |
| | | 36.03% to 72.68% | | 58.84% to 81.31% | |
| > 246.8 | 54.84 | | 71.01 | | 1.892 |
| | | 36.03% to 72.68% | | 60.38% to 82.54% | |
| > 248.8 | 54.84 | | 72.46 | | 1.992 |
| | | 33.06% to 69.85% | | 60.38% to 82.54% | |
| > 251.7 | 51.61 | | 72.46 | | 1.874 |
| | | 33.06% to 69.85% | | 61.94% to 83.75% | |
| > 255.4 | 51.61 | | 73.91 | | 1.978 |
| | | 33.06% to 69.85% | | 63.51% to 84.95% | |
| > 258.5 | 51.61 | | 75.36 | | 2.095 |
| | | 33.06% to 69.85% | | 65.09% to 86.13% | |
| > 260.2 | 51.61 | | 76.81 | | 2.226 |
| | | 30.15% to 66.94% | | 65.09% to 86.13% | |
| > 267.9 | 48.39 | | 76.81 | | 2.087 |
| | | 30.15% to 66.94% | | 66.69% to 87.29% | |
| > 276.4 | 48.39 | | 78.26 | | 2.226 |
| | | 30.15% to 66.94% | | 68.31% to 88.44% | |
| > 278.7 | 48.39 | | 79.71 | | 2.385 |
| | | 30.15% to 66.94% | | 69.94% to 89.57% | |
| > 281.6 | 48.39 | | 81.16 | | 2.568 |
| | | 30.15% to 66.94% | | 71.59% to 90.68% | |
| > 283.7 | 48.39 | | 82.61 | | 2.782 |
| | | 30.15% to 66.94% | | 73.26% to 91.76% | |
| > 285.8 | 48.39 | | 84.06 | | 3.035 |
| | | 30.15% to 66.94% | | 74.96% to 92.83% | |
| > 288.5 | 48.39 | | 85.51 | | 3.339 |
| | | 30.15% to 66.94% | | 76.68% to 93.86% | |
| > 290.1 | 48.39 | | 86.96 | | 3.71 |
| | | 27.32% to 63.97% | | 76.68% to 93.86% | |
| > 292.5 | 45.16 | | 86.96 | | 3.462 |
| | | 24.55% to 60.92% | | 76.68% to 93.86% | |
| > 295.3 | 41.94 | | 86.96 | | 3.215 |
| | | 21.85% to 57.81% | | 76.68% to 93.86% | |
| > 296.8 | 38.71 | | 86.96 | | 2.968 |
| | | 19.23% to 54.63% | | 76.68% to 93.86% | |
| > 299.5 | 35.48 | | 86.96 | | 2.72 |
| | | 19.23% to 54.63% | | 78.43% to 94.86% | |
| > 302.9 | 35.48 | | 88.41 | | 3.06 |
| | | 19.23% to 54.63% | | 80.21% to 95.82% | |
| > 305.4 | 35.48 | | 89.86 | | 3.498 |
| | | 19.23% to 54.63% | | 82.03% to 96.74% | |
| > 309.4 | 35.48 | | 91.3 | | 4.081 |
| | | 19.23% to 54.63% | | 83.89% to 97.61% | |
| > 313.4 | 35.48 | | 92.75 | | 4.897 |
| | | 16.68% to 51.37% | | 83.89% to 97.61% | |
| > 320.4 | 32.26 | | 92.75 | | 4.452 |
| | | 16.68% to 51.37% | | | |
| > 327.9 | 32.26 | | 94.2 | 85.82% to 98.4% | 5.565 |
| | | 16.68% to 51.37% | | 87.82% to 99.09% | |
| > 333.1 | 32.26 | | 95.65 | | 7.419 |
| | | 14.22% to 48.04% | | 87.82% to 99.09% | |
| > 340.1 | 29.03 | | 95.65 | | 6.677 |
| | | 14.22% to 48.04% | | 89.92% to 99.65% | |
| > 343.7 | 29.03 | | 97.1 | | 10.02 |
| | | 11.86% to 44.61% | | 89.92% to 99.65% | |
| > 346.4 | 25.81 | | 97.1 | | 8.903 |
| | | 9.594% to 41.1% | | 89.92% to 99.65% | |
| > 349.3 | 22.58 | | 97.1 | | 7.79 |
| | | 7.452% to 37.47% | | 89.92% to 99.65% | |
| > 367 | 19.35 | | 97.1 | | 6.677 |
| | | 7.452% to 37.47% | | 92.19% to 99.96% | |
| > 390.1 | 19.35 | | 98.55 | | 13.35 |
| | | 5.452% to 33.73% | | 92.19% to 99.96% | |
| > 397.5 | 16.13 | | 98.55 | | 11.13 |
| | | 3.63% to 29.83% | | 92.19% to 99.96% | |
| > 402.6 | 12.9 | | 98.55 | | 8.903 |
| | | 2.042% to 25.75% | | 92.19% to 99.96% | |
| > 410.8 | 9.677 | | 98.55 | | 6.677 |
| | | 2.042% to 25.75% | | | |
| > 415.7 | 9.677 | | 100 | 94.79% to 100% | |
| | | 0.7911% to 21.42% | | | |
| > 423.8 | 6.452 | | 100 | 94.79% to 100% | |
| | | 0.08164% to 16.7% | | | |
| > 547.9 | 3.226 | | 100 | 94.79% to 100% | |

TABLE 26

Cut-off point analyses for IL-1beta in serum

| | | | | | Likelihood ratio |
|---|---|---|---|---|---|
| Cutoff | Sensitivity% | 95% CI | Specificity% | 95% CI | |
| | | 0.2809% to 48.25% | | 55.5% to 99.75% | |
| < 0.391 | 11.11 | | 90 | | 1.111 |
| | | 2.814% to 60.01% | | 55.5% to 99.75% | |
| < 0.3965 | 22.22 | | 90 | | 2.222 |
| | | 7.485% to 70.07% | | 55.5% to 99.75% | |
| < 0.4105 | 33.33 | | 90 | | 3.333 |
| | | 7.485% to 70.07% | | 44.39% to 97.48% | |
| < 0.434 | 33.33 | | 80 | | 1.667 |
| | | 13.7% to 78.8% | | 44.39% to 97.48% | |
| < 0.5085 | 44.44 | | 80 | | 2.222 |
| | | 13.7% to 78.8% | | 34.75% to 93.33% | |
| < 0.573 | 44.44 | | 70 | | 1.481 |
| | | 13.7% to 78.8% | | 26.24% to 87.84% | |
| < 0.596 | 44.44 | | 60 | | 1.111 |
| | | 21.2% to 86.3% | | 26.24% to 87.84% | |
| < 0.6165 | 55.56 | | 60 | | 1.389 |
| | | 21.2% to 86.3% | | 18.71% to 81.29% | |
| < 0.644 | 55.56 | | 50 | | 1.111 |
| | | 29.93% to 92.51% | | 18.71% to 81.29% | |
| < 0.684 | 66.67 | | 50 | | 1.333 |
| | | 29.93% to 92.51% | | 12.16% to 73.76% | |
| < 0.712 | 66.67 | | 40 | | 1.111 |
| | | 39.99% to 97.19% | | 12.16% to 73.76% | |
| < 0.791 | 77.78 | | 40 | | 1.296 |
| | | 39.99% to 97.19% | | 6.674% to 65.25% | |
| < 0.8585 | 77.78 | | 30 | | 1.111 |
| | | 51.75% to 99.72% | | 6.674% to 65.25% | |
| < 0.8685 | 88.89 | | 30 | | 1.27 |
| | | 51.75% to 99.72% | | 2.521% to 55.61% | |
| < 1 | 88.89 | | 20 | | 1.111 |
| | | 66.37% to 100% | | 2.521% to 55.61% | |
| < 1.436 | 100 | | 20 | | 1.25 |
| | | 66.37% to 100% | | 0.2529% to 44.5% | |
| < 1.822 | 100 | | 10 | | 1.111 |

### Incorporation by Reference

The following references are incorporated by reference in their entireties for all purposes.

1.) Biomarkers Definitions Working G. Biomarkers and surrogate endpoints: preferred definitions and conceptual framework. Clin Pharmacol Ther. 2001;69:89-95.

2.) Brand FJ, 3rd, Forouzandeh M, Kaur H, Travascio F, & de Rivero Vaccari JP (2016) Acidification changes affect the inflammasome in human nucleus pulposus cells. J Inflamm (Lond) 13(1):29.

3.) Keane RW, Dietrich WD, & de Rivero Vaccari JP (2018) Inflammasome Proteins As Biomarkers of Multiple Sclerosis. Front Neurol 9:135.

### Numbered Embodiments of the Disclosure

Other subject matter contemplated by the present disclosure is set out in the following numbered embodiments:

1. A method of evaluating a patient suspected of having multiple sclerosis (MS), the method comprising: measuring the level of at least one inflammasome protein in a biological sample obtained from the patient; determining the presence or absence of a protein signature associated with MS, wherein the protein signature comprises an elevated level of the at least one inflammasome protein; and selecting the patient as having MS if the patient exhibits the presence of the protein signature.

2. The method of embodiment 1, wherein the patient is presenting with clinical symptoms consistent with MS.

3. The method of embodiment 1 or 2, wherein the MS is relapsing-remitting MS (RRMS), secondary-progressive MS (SPMS), primary-progressive MS (PPMS), or progressive-relapsing MS (PRMS).

4. The method of any one of the above embodiments, wherein the biological sample obtained from the patient is cerebrospinal fluid (CSF), CNS microdialysate, saliva, serum, plasma, urine or serum-derived extracellular vesicles (EVs).

5. The method of any one of the above embodiments, wherein the level of the at least one inflammasome protein in the protein signature is measured by an immunoassay utilizing one or more antibodies directed against the at least one inflammasome protein in the protein signature.

6. The method of any one of the above embodiments, wherein the at least one inflammasome protein is interleukin 18 (IL-18), IL-1beta, apoptosis-associated speck-like protein containing a caspase recruitment domain (ASC), caspase-1, or combinations thereof.

7. The method of any of the above embodiments, wherein the at least one inflammasome protein comprises each of caspase-1, IL-18, IL-1beta and ASC.

8. The method of any one of embodiments 1-6, wherein the at least one inflammasome protein comprises ASC.

9. The method of any one of embodiments 5-8, wherein the antibody binds to the PYRIN-PAAD-DAPIN domain (PYD), C-terminal caspase-recruitment domain (CARD) domain or a portion of the PYD or CARD domain of the ASC protein.

10. The method of any one of the above embodiments, wherein the level of the at least one inflammasome protein in the protein signature is enhanced relative to the level of the at least one inflammasome protein in a biological sample obtained from a control.

11. The method of embodiment 10, wherein the biological sample obtained from the control is cerebrospinal fluid (CSF), CNS microdialysate, saliva, serum, plasma, urine or serum-derived extracellular vesicles (EVs).

12. The method of embodiment 10 or 11, wherein the control is a healthy individual, wherein the healthy individual is an individual not presenting with clinical symptoms consistent with MS.

13. The method of any one of embodiments 10-12, wherein the at least one inflammasome protein comprises ASC, wherein the level of ASC is at least 50% higher than the level of ASC in the biological sample obtained from a control.

14. The method of any one of embodiments 1-9, wherein the level of the at least one inflammasome protein in the protein signature is enhanced relative to a pre-determined reference value or range of reference values.

15. The method of embodiment 14, wherein the biological sample obtained from patient is serum and the patient is selected as having MS with a sensitivity of at least 80%, 85%, 90%, 95%, 99% or 100% and a specificity of at least 90%.

16. The method of embodiment 14 or 15, wherein the biological sample is serum and the patient is selected as having MS with a specificity of at least 80%, 85%, 90%, 95%, 99% or 100%.

17. The method of embodiment 14, wherein the biological sample is serum and the patient is selected as having MS with a sensitivity of at least 90% and a specificity of at least 80%.

18. The method of any one of embodiments 14-17, wherein the at least one inflammasome protein comprises ASC.

19. The method of embodiment 18, wherein a cut-off value for determining the sensitivity, specificity or both is selected from Table 7.

20. The method of any one of embodiments 15-17, wherein the sensitivity and/or sensitivity is determined using the area under curve (AUC) from receiver operator characteristic (ROC) curves with confidence intervals of 95%.

21. A method of evaluating a patient suspected of having suffered a stroke, the method comprising: measuring the level of at least one inflammasome protein in a biological sample obtained from the patient; determining the presence or absence of a protein signature associated with stroke or a stroke-related injury, wherein the protein signature comprises an elevated level of the at least one inflammasome protein; and selecting the patient as having suffered from a stroke if the patient exhibits the presence of the protein signature.

22. The method of embodiment 21, wherein the patient is presenting with clinical symptoms consistent with stroke, wherein the stroke is ischemic stroke, transient ischemic stroke or hemorrhagic stroke.

23. The method of embodiment 21 or 22, wherein the biological sample obtained from the patient is cerebrospinal fluid (CSF), CNS microdialysate, saliva, serum, plasma, urine or serum-derived extracellular vesicles (EVs).

24. The method of any one of embodiments 21-23, wherein the level of the at least one inflammasome protein in the protein signature is measured by an immunoassay utilizing one or more antibodies directed against the at least one inflammasome protein in the protein signature.

25. The method of any one of embodiments 21-24, wherein the at least one inflammasome protein is interleukin 18 (IL-18), IL-1beta, apoptosis-associated speck-like protein containing a caspase recruitment domain (ASC), caspase-1, or combinations thereof.

26. The method of any of embodiments 21-25, wherein the at least one inflammasome protein comprises each of caspase-1, IL-18, IL-1beta and ASC.

27. The method of any one of embodiments 21-25, wherein the at least one inflammasome protein comprises ASC.

28. The method of any one of embodiments 25-27, wherein the antibody binds to the PYRIN-PAAD-DAPIN domain (PYD), C-terminal caspase-recruitment domain (CARD) domain or a portion of the PYD or CARD domain of the ASC protein.

29. The method of any one of embodiments 21-28, wherein the level of the at least one inflammasome protein in the protein signature is enhanced relative to the level of the at least one inflammasome protein in a biological sample obtained from a control.

30. The method of embodiment 29, wherein the biological sample obtained from the control is cerebrospinal fluid (CSF), CNS microdialysate, saliva, serum, plasma, urine or serum-derived extracellular vesicles (EVs).

31. The method of embodiment 29 or 30, wherein the control is a healthy individual, wherein the healthy individual is an individual not presenting with clinical symptoms consistent with MS.

32. The method of any one of embodiments 29-31, wherein the at least one inflammasome protein comprises ASC, wherein the level of ASC in a serum sample obtained from the subject is at least 70% higher than the level of ASC in a serum sample obtained from a control.

33. The method of any one of embodiments 29-31, wherein the at least one inflammasome protein comprises ASC, wherein the level of ASC in a serum-derived EV sample obtained from the subject is at least 110% higher than the level of ASC in a serum-derived EV sample obtained from a control.

34. The method of any one of embodiments 21-28, wherein the level of the at least one inflammasome protein in the protein signature is enhanced relative to a pre-determined reference value or range of reference values.

35. The method of embodiment 34, wherein the biological sample obtained from patient is serum and the patient is selected as having suffered a stroke with a sensitivity of at least 80%, 85%, 90%, 95%, 99% or 100% and a specificity of at least 90%.

36. The method of embodiment 34 or 35, wherein the biological sample is serum and the patient is selected as having suffered a stroke with a specificity of at least 80%, 85%, 90%, 95%, 99% or 100%.

37. The method of embodiment 34, wherein the biological sample is serum and the patient is selected as having suffered a stroke with a sensitivity of at least 100% and a specificity of at least 95%.

38. The method of any one of embodiments 35-37, wherein the at least one inflammasome protein comprises ASC.

39. The method of embodiment 38, wherein a cut-off value for determining the sensitivity, specificity or both is selected from Table 8.

40. The method of embodiment 34, wherein the biological sample obtained from patient is serum-derived EVs and the patient is selected as having suffered a stroke with a sensitivity of at least 80%, 85%, 90%, 95%, 99% or 100% and a specificity of at least 90%.

41. The method of embodiment 34 or 40, wherein the biological sample is serum-derived EVs and the patient is selected as having suffered a stroke with a specificity of at least 80%, 85%, 90%, 95%, 99% or 100%.

42. The method of embodiment 34, wherein the biological sample is serum-derived EVs and the patient is selected as having suffered a stroke with a sensitivity of at least 100% and a specificity of at least 100%.

43. The method of any one of embodiments 40-42, wherein the at least one inflammasome protein comprises ASC.

44. The method of embodiment 43, wherein a cut-off value for determining the sensitivity, specificity or both is selected from Table 9.

45. The method of any one of embodiments 35-37 or 40-42, wherein the sensitivity and/or sensitivity is determined using the area under curve (AUC) from receiver operator characteristic (ROC) curves with confidence intervals of 95%.

46. A method of treating a patient diagnosed with multiple sclerosis (MS), the method comprising administering a standard of care treatment for MS to the patient, wherein the diagnosis of MS was made by detecting an elevated level of at least one inflammasome protein in a biological sample obtained from the patient.

47. The method of embodiment 46, wherein the MS is relapsing-remitting MS (RRMS), secondary-progressive MS (SPMS), primary-progressive MS (PPMS), or progressive-relapsing MS (PRMS).

48. The method of embodiment 46 or 47, wherein the standard of care treatment is selected from therapies directed towards modifying disease outcome, managing relapses, managing symptoms or any combination thereof.

49. The method of embodiment 48, wherein the therapies directed toward modifying disease outcome are selected from beta-interferons, glatiramer acetate, fingolimod, teriflunomide, dimethyl fumarate, mitoxanthrone, ocrelizumab, alemtuzumab, daclizumab and natalizumab.

50. A method of treating a patient diagnosed with stroke or a stroke related injury, the method comprising administering a standard of care treatment for stroke or stroke-related injury to the patient, wherein the diagnosis of stroke or stroke-related injury was made by detecting an elevated level of at least one inflammasome protein in a biological sample obtained from the patient.

51. The method of embodiment 50, wherein the stroke is ischemic stroke, transient ischemic stroke or hemorrhagic stroke.

52. The method of embodiment 50 or 51, wherein the stroke is ischemic stroke or transient ischemic stroke and the standard of care treatment is selected from tissue plasminogen activator (tPA), antiplatelet medicine, anticoagulants, a carotid artery angioplasty, carotid endarterectomy, intra-arterial thrombolysis and mechanical clot removal in cerebral ischemia (MERCI) or a combination thereof.

53. The method of embodiment 50 or 51, wherein the stroke is hemorrhagic stroke and the standard of care treatment is an aneurysm clipping, coil embolization or arteriovenous malformation (AVM) repair.

54. The method of any one of embodiments 46-53, wherein the elevated level of the at least one inflammasome protein is measured by an immunoassay utilizing one or more antibodies directed against the at least one inflammasome protein.

55. The method of any one of embodiments 46-54, wherein the level of the at least one inflammasome protein is enhanced relative to the level of the at least one inflammasome protein in a control sample.

56. The method of any one of embodiments 46-54, wherein the level of the at least one inflammasome protein is enhanced relative to a pre-determined reference value or range of reference values.

57. The method of any one of embodiments 46-56, wherein the at least one inflammasome protein is interleukin 18 (IL-18), apoptosis-associated speck-like protein containing a caspase recruitment domain (ASC), caspase-1, or combinations thereof.

58. The method of embodiment 56 or 57, wherein the at least one inflammasome protein is caspase-1, IL-18, and ASC.

59. The method of embodiment 56 or 57, wherein the at least one inflammasome protein is ASC.

60. The method of embodiment 59, wherein the antibody binds to the PYRIN-PAAD-DAPIN domain (PYD), C-terminal caspase-recruitment domain (CARD) domain or a portion of the PYD or CARD domain of the ASC protein.

61. The method of any one of embodiments 46-60, wherein the biological sample is cerebrospinal fluid (CSF), CNS microdialysate, saliva, serum, plasma, urine or serum-derived extracellular vesicles (EVs).

62. A method of evaluating a patient suspected of having traumatic brain injury (TBI), the method comprising: measuring the level of at least one inflammasome protein in a biological sample obtained from the patient; determining the presence or absence of a protein signature associated with TBI, wherein the protein signature comprises an elevated level of the at least one inflammasome protein; and selecting the patient as having TBI if the patient exhibits the presence of the protein signature.

63. The method of embodiment 62, wherein the patient is presenting with clinical symptoms consistent with TBI.

64. The method of embodiment 62 or 63, wherein the biological sample obtained from the patient is cerebrospinal fluid (CSF), CNS microdialysate, saliva, serum, plasma, urine or serum-derived extracellular vesicles (EVs).

65. The method of any one of embodiments 62-64, wherein the level of the at least one inflammasome protein in the protein signature is measured by an immunoassay utilizing one or more antibodies directed against the at least one inflammasome protein in the protein signature.

66. The method of any one of embodiments 62-65, wherein the at least one inflammasome protein is interleukin 18 (IL-18), IL-1β, apoptosis-associated speck-like protein containing a caspase recruitment domain (ASC), caspase-1, or combinations thereof.

67. The method of any one of embodiments 61-66, wherein the at least one inflammasome protein comprises caspase-1.

The method of any one of embodiments 65-67, wherein the at least one inflammasome protein comprises caspase-1, wherein the level of caspase-1is at least 50% higher than the level of caspase-1in the biological sample obtained from a control.

68. The method of any one of embodiments 61-66, wherein the at least one inflammasome protein comprises ASC.

69. The method of any one of embodiments 66 or 68, wherein the antibody binds to the PYRIN-PAAD-DAPIN domain (PYD), C-terminal caspase-recruitment domain (CARD) domain or a portion of the PYD or CARD domain of the ASC protein.

70. The method of any one of embodiments 62-69, wherein the level of the at least one inflammasome protein in the protein signature is enhanced relative to the level of the at least one inflammasome protein in a biological sample obtained from a control.

71. The method of embodiment 70, wherein the at least one inflammasome protein comprises caspase-1, wherein the level of caspase-1 is at least 50% higher than the level of caspase-1in the biological sample obtained from the control.

72. The method of embodiment 70, wherein the at least one inflammasome protein comprises ASC, wherein the level of ASC is at least 50% higher than the level of ASC in the biological sample obtained from athe control.

73. The method of any one of embodiments 70-72, wherein the biological sample obtained from the control is cerebrospinal fluid (CSF), CNS microdialysate, saliva, serum, plasma, urine or serum-derived extracellular vesicles (EVs).

74. The method of any one of embodiments 70-73, wherein the control is a healthy individual, wherein the healthy individual is an individual not presenting with clinical symptoms consistent with TBI.

75. The method of any one of embodiments 62-69, wherein the level of the at least one inflammasome protein in the protein signature is enhanced relative to a pre-determined reference value or range of reference values.

76. The method of embodiment 75, wherein the biological sample obtained from patient is serum and the patient is selected as having TBI with a sensitivity of at least 80%, 85%, 90%, 95%, 99% or 100% and a specificity of at least 90%.

77. The method of embodiment 75 or 76, wherein the biological sample is serum and the patient is selected as having TBI with a specificity of at least 80%, 85%, 90%, 95%, 99% or 100%.

78. The method of embodiment 75, wherein the biological sample is serum and the patient is selected as having TBI with a sensitivity of at least 90% and a specificity of at least 80%.

79. The method of any one of embodiments 76-76, wherein the sensitivity and/or sensitivity is determined using the area under curve (AUC) from receiver operator characteristic (ROC) curves with confidence intervals of 95%.

80. The method of any one of embodiments 75-79, wherein the at least one inflammasome protein comprises ASC.

81. The method of embodiment 79, wherein a cut-off value for determining the sensitivity, specificity or both is selected from Tables 11B, 12B, 14A, 16, 17 or 19.

82. The method of any one of embodiments 75-79, wherein the at least one inflammasome protein comprises caspase-1.

83. The method of embodiment 82, wherein a cut-off value for determining the sensitivity, specificity or both is selected from Tables 11A or 15.

84. A method of evaluating a patient suspected of having a brain injury, the method comprising: measuring the level of at least one inflammasome protein in a biological sample obtained from the patient; determining the presence or absence of a protein signature associated with brain injury, wherein the protein signature comprises an elevated level of the at least one inflammasome protein; and selecting the patient as having brain injury if the patient exhibits the presence of the protein signature.

85. The method of embodiment 84, wherein the patient is presenting with clinical symptoms consistent with brain injury.

86. The method of embodiment 84 or 85, wherein the biological sample obtained from the patient is cerebrospinal fluid (CSF), CNS microdialysate, saliva, serum, plasma, urine or serum-derived extracellular vesicles (EVs).

87. The method of any one of embodiments 84-86, wherein the level of the at least one inflammasome protein in the protein signature is measured by an immunoassay utilizing one or more antibodies directed against the at least one inflammasome protein in the protein signature.

88. The method of any one of embodiments 84-87, wherein the at least one inflammasome protein is interleukin 18 (IL-18), IL-1β, apoptosis-associated speck-like protein containing a caspase recruitment domain (ASC), caspase-1, or combinations thereof.

89. The method of any one of embodiments 84-88, wherein the at least one inflammasome protein comprises ASC.

90. The method of embodiment 88 or 89, wherein the antibody binds to the PYRIN-PAAD-DAPIN domain (PYD), C-terminal caspase-recruitment domain (CARD) domain or a portion of the PYD or CARD domain of the ASC protein.

91. The method of any of embodiments 84-88, wherein the at least one inflammasome protein comprises caspase-1.

92. The method of any one of embodiments 84-91, wherein the level of the at least one inflammasome protein in the protein signature is enhanced relative to the level of the at least one inflammasome protein in a biological sample obtained from a control.

93. The method of embodiment 92, wherein the at least one inflammasome protein comprises ASC, wherein the level of ASC is at least 50% higher than the level of ASC in the biological sample obtained from the control.

94. The method of embodiment 92, wherein the at least one inflammasome protein comprises caspase-1, wherein the level of caspase-1 is at least 50% higher than the level of caspase-1in the biological sample obtained from the control.

95. The method of any one of embodiments 92-94, wherein the biological sample obtained from the control is cerebrospinal fluid (CSF), CNS microdialysate, saliva, serum, plasma, urine or serum-derived extracellular vesicles (EVs).

96. The method of any one of embodiments 92-95, wherein the control is a healthy individual, wherein the healthy individual is an individual not presenting with clinical symptoms consistent with brain injury.

97. The method of any one of embodiments 84-96, wherein the brain injury is selected from a traumatic brain injury, stroke, mild cognitive impairment or multiple sclerosis.

98. The method of any one of embodiments 84-91, wherein the level of the at least one inflammasome protein in the protein signature is enhanced relative to a pre-determined reference value or range of reference values.

99. The method of embodiment 98, wherein the brain injury is traumatic brain injury (TBI).

100. The method of embodiment 99, wherein the biological sample obtained from patient is serum and the patient is selected as having TBI with a sensitivity of at least 80%, 85%, 90%, 95%, 99% or 100% and a specificity of at least 90%.

101. The method of embodiment 98 or 99, wherein the biological sample is serum and the patient is selected as having TBI with a specificity of at least 80%, 85%, 90%, 95%, 99% or 100%.

102. The method of embodiment 99, wherein the biological sample is serum and the patient is selected as having TBI with a sensitivity of at least 90% and a specificity of at least 80%.

103. The method of any one of embodiments 100-102, wherein the sensitivity and/or sensitivity is determined using the area under curve (AUC) from receiver operator characteristic (ROC) curves with confidence intervals of 95%.

104. The method of any one of embodiments 99-103, wherein the at least one inflammasome protein comprises ASC.

105. The method of embodiment 104, wherein a cut-off value for determining the sensitivity, specificity or both is selected from Tables 11B, 12B, 14A, 16, 17 or 19.

106. The method of any one of embodiments 99-103, wherein the at least one inflammasome protein comprises caspase-1.

107. The method of embodiment 106, wherein a cut-off value for determining the sensitivity, specificity or both is selected from Tables 11A or 15.

108. The method of embodiment 98, wherein the brain injury is multiple sclerosis (MS).

109. The method of embodiment 108, wherein the biological sample obtained from patient is serum and the patient is selected as having MS with a sensitivity of at least 80%, 85%, 90%, 95%, 99% or 100% and a specificity of at least 90%.

110. The method of embodiment 108 or 109, wherein the biological sample is serum and the patient is selected as having MS with a specificity of at least 80%, 85%, 90%, 95%, 99% or 100%.

111. The method of embodiment 108, wherein the biological sample is serum and the patient is selected as having MS with a sensitivity of at least 90% and a specificity of at least 80%.

112. The method of any one of embodiments 108-111, wherein the at least one inflammasome protein comprises ASC.

113. The method of embodiment 112, wherein a cut-off value for determining the sensitivity, specificity or both is selected from Table 7.

114. The method of any one of embodiments 109-113, wherein the sensitivity and/or sensitivity is determined using the area under curve (AUC) from receiver operator characteristic (ROC) curves with confidence intervals of 95%.

115. The method of embodiment 98, wherein the brain injury is stroke.

116. The method of embodiment 115, wherein the biological sample obtained from patient is serum and the patient is selected as having suffered a stroke with a sensitivity of at least 80%, 85%, 90%, 95%, 99% or 100% and a specificity of at least 90%.

117. The method of embodiment 115 or 116, wherein the biological sample is serum and the patient is selected as having suffered a stroke with a specificity of at least 80%, 85%, 90%, 95%, 99% or 100%.

118. The method of embodiment 115, wherein the biological sample is serum and the patient is selected as having suffered a stroke with a sensitivity of at least 100% and a specificity of at least 95%.

119. The method of any one of embodiments 116-118, wherein the at least one inflammasome protein comprises ASC.

120. The method of embodiment 119, wherein a cut-off value for determining the sensitivity, specificity or both is selected from Table 8.

121. The method of embodiment 115, wherein the biological sample obtained from patient is serum-derived EVs and the patient is selected as having suffered a stroke with a sensitivity of at least 80%, 85%, 90%, 95%, 99% or 100% and a specificity of at least 90%.

122. The method of embodiment 115 or 121, wherein the biological sample is serum-derived EVs and the patient is selected as having suffered a stroke with a specificity of at least 80%, 85%, 90%, 95%, 99% or 100%.

123. The method of embodiment 115, wherein the biological sample is serum-derived EVs and the patient is selected as having suffered a stroke with a sensitivity of at least 100% and a specificity of at least 100%.

124. The method of any one of embodiments 121-123, wherein the at least one inflammasome protein comprises ASC.

125. The method of embodiment 124, wherein a cut-off value for determining the sensitivity, specificity or both is selected from Table 9.

126. The method of any one of embodiments 116-118 or 121-123, wherein the sensitivity and/or sensitivity is determined using the area under curve (AUC) from receiver operator characteristic (ROC) curves with confidence intervals of 95%.

127. A method of evaluating a patient suspected of having mild cognitive impairment (MCI) the method comprising: measuring the level of at least one inflammasome protein in a biological sample obtained from the patient; determining the presence or absence of a protein signature associated with MCI, wherein the protein signature comprises an elevated level of the at least one inflammasome protein; and selecting the patient as having MCI if the patient exhibits the presence of the protein signature.

128. The method of embodiment 127, wherein the patient is presenting with clinical symptoms consistent with MCI.

129. The method of embodiment 127 or 128, wherein the biological sample obtained from the patient is cerebrospinal fluid (CSF), CNS microdialysate, saliva, serum, plasma, urine or serum-derived extracellular vesicles (EVs).

130. The method of any one of embodiments 127-129, wherein the level of the at least one inflammasome protein in the protein signature is measured by an immunoassay utilizing one or more antibodies directed against the at least one inflammasome protein in the protein signature.

131. The method of any one of embodiments 127-130, wherein the at least one inflammasome protein is interleukin 18 (IL-18), IL-1β, apoptosis-associated speck-like protein containing a caspase recruitment domain (ASC), caspase-1, or combinations thereof.

132. The method of any one of embodiments 127-131, wherein the at least one inflammasome protein comprises ASC.

133. The method of any one of embodiments 127-131, wherein the at least one inflammasome protein comprises IL-18.

134. The method of any one of embodiments 131-132, wherein the antibody binds to the PYRIN-PAAD-DAPIN domain (PYD), C-terminal caspase-recruitment domain (CARD) domain or a portion of the PYD or CARD domain of the ASC protein.

135. The method of any one of embodiments 127-134, wherein the level of the at least one inflammasome protein in the protein signature is enhanced relative to the level of the at least one inflammasome protein in a biological sample obtained from a control.

136. The method of embodiment 135, wherein the at least one inflammasome protein comprises ASC, wherein the level of ASC is at least 50% higher than the level of ASC in the biological sample obtained from the control.

137. The method of embodiment 135, wherein the at least one inflammasome protein comprises IL-18, wherein the level of IL-18 is at least 25% higher than the level of IL-18 in the biological sample obtained from the control.

138. The method of any one of embodiments 135-137, wherein the biological sample obtained from the control is cerebrospinal fluid (CSF), CNS microdialysate, saliva, serum, plasma, urine or serum-derived extracellular vesicles (EVs).

139. The method of any one of embodiments 135-138, wherein the control is a healthy individual, wherein the healthy individual is an individual not presenting with clinical symptoms consistent with MCI.

140. The method of any one of embodiments 127-134, wherein the level of the at least one inflammasome protein in the protein signature is enhanced relative to a pre-determined reference value or range of reference values.

141. The method of embodiment 140, wherein the biological sample obtained from patient is serum and the patient is selected as having MCI with a sensitivity of at least 70%, 75%, 80%, 85%, 90%, 95%, 99% or 100% and a specificity of at least 55%.

142. The method of embodiment 140 or 141, wherein the biological sample is serum and the patient is selected as having MCI with a sensitivity of at least 70%, 75%, 80%, 85%, 90%, 95%, 99% or 100%.

143. The method of embodiment 140, wherein the biological sample is serum and the patient is selected as having MCI with a sensitivity of at least 70% and a specificity of at least 55%.

144. The method of any one of embodiments 140-143, wherein the sensitivity and/or sensitivity is determined using the area under curve (AUC) from receiver operator characteristic (ROC) curves with confidence intervals of 95%.

145. The method of any one of embodiments 140-144, wherein the at least one inflammasome protein comprises ASC.

146. The method of embodiment 145, wherein a cut-off value for determining the sensitivity, specificity or both is selected from Table 22.

147. The method of any one of embodiments 140-144, wherein the at least one inflammasome protein comprises IL-18.

148. The method of embodiment 147, wherein a cut-off value for determining the sensitivity, specificity or both is selected from Table 22.

The various embodiments described above can be combined to provide further embodiments. All of the U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified, if necessary to employ concepts of the various patents, application and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1-49. (canceled)

50. A method of treating a patient with stroke, the method comprising: (a) measuring an expression level of at least one inflammasome protein in a biological sample obtained from a patient,
(b) comparing the expression level of the at least one inflammasome protein in the biological sample obtained from the patient to a control;
(c) diagnosing the patient with stroke when the expression level of the at least one inflammasome protein in the biological sample obtained from the patient is enhanced relative to the control; and
(d) administering a standard of care treatment for stroke or stroke-related injury to the patient.

51. The method of claim 50, wherein the stroke is ischemic stroke, transient ischemic stroke or hemorrhagic stroke.

52. The method of claim 50, wherein the stroke is ischemic stroke or transient ischemic stroke and the standard of care treatment is selected from tissue plasminogen activator (tPA), antiplatelet medicine, anticoagulants, a carotid artery angioplasty, carotid endarterectomy, intraarterial thrombolysis and mechanical clot removal in cerebral ischemia (MERCI) or a combination thereof.

53. The method of claim 50, wherein the stroke is hemorrhagic stroke and the standard of care treatment is an aneurysm clipping, coil embolization or arteriovenous malformation (AVM) repair.

54. The method of 46, wherein the enhanced level of the at least one inflammasome protein is measured by an immunoassay utilizing one or more antibodies directed against the at least one inflammasome protein.

55. (canceled)

56. The method of claim 54, wherein the control is a pre-determined reference value or range of reference values.

57. The method of claim 56, wherein the at least one inflammasome protein is selected from the group consisting of interleukin 18 (IL-18), apoptosis-associated speck-like protein containing a caspase recruitment domain (ASC), caspase-1, and combinations thereof.

58. The method of claim 56, wherein the at least one inflammasome protein is caspase-1, IL-18, and ASC.

59. The method of claim 56, wherein the at least one inflammasome protein is ASC, wherein the pre-determined reference value is a cut-off value is selected from Table 7.

60. The method of claim 59, wherein the antibody binds to the PYRIN-PAAD-DAPIN domain (PYD), C-terminal caspase-recruitment domain (CARD) domain or a portion of the PYD or CARD domain of the ASC protein.

61. The method of claim 54, wherein the biological sample is cerebrospinal fluid (CSF), CNS microdialysate, saliva, serum, plasma, urine or serum-derived extracellular vesicles (EVs).

62-148. (canceled)

149. The method of claim 50, wherein the enhanced level of the at least one inflammasome protein is measured by an immunoassay utilizing one or more antibodies directed against the at least one inflammasome protein.

150. The method of claim 50, wherein the at least one inflammasome protein comprises ASC, wherein the level of ASC is at least 70% higher than the level of ASC in the biological sample obtained from a control.

151. The method of claim 50, wherein the at least one inflammasome protein comprises ASC, wherein the biological sample is a serum-derived EV sample, wherein the level of ASC in the serum-derived EV sample obtained from the subject is at least 110% higher than the level of ASC in a serum-derived EV sample obtained from a control.

152. The method of claim 50, wherein the control is a pre-determined reference value or range of reference values.

153. The method of claim 50, wherein the at least one inflammasome protein is ASC, wherein the pre-determined reference value is a cut-off value is selected from Table 8.

154. The method of claim 50, wherein the at least one inflammasome protein is ASC and the biological sample is serum-derived EVs, wherein the pre-determined reference value is a cut-off value is selected from Table 9.

155. The method of claim 50, wherein the at least one inflammasome protein is selected from the group consisting of interleukin 18 (IL-18), IL-lbeta, apoptosis-associated speck-like protein containing a caspase recruitment domain (ASC), caspase-1, and combinations thereof.


SEQUENCE LISTING (4 sequences)

SEQ ID NO: 1 - protein, 16 aa, Rattus sp.
ALRQTQPYLVTDLEQS

SEQ ID NO: 2 - protein, 14 aa, Homo sapiens
RESQSYLVEDLERS

SEQ ID NO: 3 - protein, 19 aa, Homo sapiens
CEYYTEIREREREKSEKGR

SEQ ID NO: 4 - protein, 15 aa, Rattus sp.
MEESQSKEESNTEGC